(19) United States
(12) Patent Application Publication
(10) Pub. No.: US 2022/0317115 A1
(43) Pub. Date: Oct. 6, 2022

(54) 3D ORGANOIDS FOR PERSONALIZED ORAL CANCER THERAPY

(71) Applicant: THE TRUSTEES OF COLUMBIA UNIVERSITY IN THE CITY OF NEW YOURK, New York, NY (US)

(72) Inventors: Hiroshi NAKAGAWA, New York, NY (US); Angela J. YOON, New York, NY (US); Anil K. RUSTGI, Villanova, PA (US)

(21) Appl. No.: 17/835,786

(22) Filed: Jun. 8, 2022

Related U.S. Application Data
(63) Continuation of application No. PCT/US2020/064035, filed on Dec. 9, 2020.
(60) Provisional application No. 62/975,641, filed on Feb. 12, 2020.
(60) Provisional application No. 62/945,858, filed on Dec. 9, 2019.

(51) Int. Cl. G01N 33/50; C12N 5/09; G01N 33/574; C12Q 1/6886; A61K 33/243; A61K 31/513; A61K 31/265; A61P 35/00

(52) U.S. Cl. CPC G01N 33/5085; C12N 5/0693; G01N 33/57407; C12Q 1/6886; A61K 33/243; A61K 31/513; A61K 31/265; A61P 35/00; C12N 2513/00; C12Q 2600/106; C12Q 2600/156; C12Q 2600/118

(57) ABSTRACT

The present disclosure provides, inter alia, compositions and methods for treating or ameliorating the effects of a tumor in a subject comprising a three-dimensional (3D) organoid system.

### CROSS REFERENCE TO RELATED APPLICATIONS

The present application is a continuation of PCT international application no. PCT/US2020/064035, filed on Dec. 9, 2020, which claims benefit of U.S. Provisional Patent Application Ser. No. 62/975,641, filed on Feb. 12, 2020, and U.S. Provisional Patent Application Ser. No. 62/945,858, filed on Dec. 9, 2019, which applications are incorporated by reference herein in their entireties.

### GOVERNMENT FUNDING

This invention was made with government support under grant nos. AA026297, CA098101, DK114436, CA163004 and DE026801 awarded by the National Institutes of Health. The government has certain rights in the invention.

### FIELD OF DISCLOSURE

The present disclosure provides, inter alia, compositions and methods for treating or ameliorating the effects of a tumor in a subject comprising a three-dimensional (3D) organoid system.

### BACKGROUND OF THE DISCLOSURE

Oral squamous cell carcinoma (OSCC) is a deadly disease, common worldwide while accounting for ˜7,400 deaths each year in the United States. The majority of OSCC develop from oral preneoplasia (OP). Despite the improvement in knowledge pertaining to OSCC as well as advances in diagnostic approaches, prognostication and treatment, the relative 5-year survival rate of OSCC remains at approximately 50%. Thus, there is a need to identify genetic factors that can be manipulated for prevention or treatment of OSCC, and to develop translatable platforms that permit risk assessment, molecular subtyping and timely exploration of therapeutic options specific for each patient with OP and OSCC to deliver personalized therapy, thereby improving overall survival.

### SUMMARY OF THE DISCLOSURE

One embodiment of the present disclosure is a method for stratifying the risk of developing a tumor in a subject, comprising: obtaining a biological sample from the subject; generating a three-dimensional (3D) organoid system from the biological sample; and detecting one or more dysplastic 3D structures.

Another embodiment of the present disclosure is a method for treating or ameliorating the effects of a tumor in a subject, comprising: obtaining a biological sample from the subject; generating a three-dimensional (3D) organoid system from the biological sample; determining the aldehyde dehydrogenase (Aldh)-2 genotype of the subject using the 3D organoid system; and administering to the subject with an effective amount of a chemotherapy agent, if the Aldh2 genotype is Aldh2^{E487K}.

Another embodiment of the present disclosure is a method for improving the efficacy of chemotherapy in a subject with a tumor, comprising obtaining a biological sample from the subject; generating a three-dimensional (3D) organoid system from the biological sample; determining the aldehyde dehydrogenase (Aldh)-2 genotype of the subject using the 3D organoid system; and co-administering to the subject with an effective amount of a chemotherapy agent and an effective amount of an agent that inhibits Aldh2, if the Aldh2 genotype is Aldh2^{E487K}.

Another embodiment of the present disclosure is a method of treating or ameliorating the effects of an oral tumor in a subject comprising the steps of: detecting the presence of the aldehyde dehydrogenase 2 (Aldh2) single nucleotide polymorphism (SNP) Aldh2^{E487K} in the subject; and administering a chemotherapy agent if Aldh2^{E487K} is detected.

Another embodiment of the present disclosure is a method of treating or ameliorating the effects of an oral tumor comprising the steps of: detecting the presence of Aldh2^{E487K}; and administering an Aldh2 inhibitor and a chemotherapy agent.

Still another embodiment of the present disclosure is a method of screening for efficacy of a chemotherapy agent against a tumor comprising the steps of: obtaining a biological sample from a subject; generating a three-dimensional (3D) organoid system from the biological sample; and contacting one or more cells of the 3D organoid system with one or more chemotherapy agents to identify efficacy of the chemotherapy agent.

### BRIEF DESCRIPTION OF THE DRAWINGS

The patent application file contains at least one drawing executed in color. Copies of this patent or patent application publication with color drawing(s) will be provided by the Office upon request and payment of the necessary fee.

FIG. 1 shows the hypothesis that ALDH2*2 promotes OSCC progression via CD44H cells that may be targeted for therapy by enhancing oxidative cell injury.

FIG. 2 shows 3D organoids from tongue biopsies from a patient with OP and adjacent normal mucosa. H&E staining. (400×)

FIG. 3 shows that Preneoplastic 3D organoids emerged from 4NQO-treated, but not untreated (Ctrl), C57/BL6 mice.

FIG. 4 shows that CD44H cells are enriched in 3D organoids from primary and metastatic tumors as determined by flow cytometry. *, p<0.05 vs. normal (n=6). LN, lymph node.

FIGS. 5A-5B show that Aldh2 SNP influences malignant transformation and chemotherapy response. 4NQO-induced tumor organoids with indicated Aldh2 SNP genotypes were analyzed by H&E staining for cellular abnormality in FIG. 5A and by flow cytometry-based CellTiter-Glo® 3D cell viability assays, following treatment for 3 days with cisplatin (80 μM) along with or without 10 μM Alda-1, an Aldh2 activator in FIG. 5B. *, p<0.01 vs. Aldh21; ns, not significant (n=6).

FIG. 6 shows 3D organoids analyses and possible treatments.

FIG. 7 shows the 4NQO protocol.

FIG. 8 shows esophageal pathologies.

FIG. 9 shows an example from mice bearing tumors induced by 4NQO, an oral-esophageal carcinogen.

FIG. 10 shows that Notch1-deleted basal cells display increased organoid formation from passage to passage. Esophageal epithelial cells isolated from Notch1loxP/loxP mice were infected with Ad-Cre/GFP or Ad-GFP (control) in single cell suspensions and allowed to grow 3D organoids (P0) and passaged (P1-P4) following FACS purification of GFP+ cells to determine OFR. *, P<0.05 vs. Ad-GFP at each passage; #, P<0.05 vs. Ad-Cre/GFP (P0), ns, vs. Ad-GFP (P0), n=6.

FIG. 11 shows that Notch1−/−3D organoids display BCH. Organoids (P4) from FIG. 10 were imaged in culture or by IHC for p63. Scale bar, 50 μm.

FIG. 12 shows that cytokine IL-13 induces BCH-like changes. 3D Organoids show an expansion of basal cells expressing p63 with downregulation of the differentiation marker IVL in response to IL-13 stimulation. Scale bars, 50 μm.

FIG. 13 shows that 4NQO induces preneoplasia and SCC in mice.

FIG. 14 shows that H&E staining reveals a lung metastatic lesion in 4NQO-treated mice with ESCC. Scale bar, 50 μm.

FIG. 15 shows the lineage tracing in mice. YFP+ esophagus with a tumor (T) and a metastatic lymph node (L) (panels a and b). IF detects YFP+ ESCC cells in the invasive tumor fronts (panel c). YFP+ cells form 3D organoids (panel d). Flow cytometry detects YFP+ CD44H cells in tumor tissues (panel e) and ESCC cells in culture (panel f). Scale bar, 50 μm.

FIG. 16 shows that ESCC organoids display EMT. H&E and multicolor IF images of murine esophageal 3D organoids. The irregular-shaped ESCC 3D organoids show Zeb1 induction in cells with concurrent E-cadherin downregulation. Box denotes area that is magnified in panel below. Scale bars, 20 μm.

FIG. 17 shows that 3D organoids recaptiulate ESCC development and progression in 4NQO model. Bar graphs depict the frequency of organoids with differential cellular atypism evaluated at variable time points. Representative organoid structures are also shown.

FIGS. 18A-18B show the metastatic ESCC 3D organoids grew from the lung and the lymph nodes. Mice were sacrificed at 24 weeks after the start of 4NQO. Phase contrast images (FIG. 18A) and average size (FIG. 18B) of 0 organoids from indicated group. *, p<0.01 vs. ESCC 3D organoids from the esophagus containing primary tumors.

FIG. 19 shows human esophageal 3D organoids from BE and adjacent normal mucosa. Biopsies and 3D organoid culture products were stained with Alcian blue to document goblet cells. Note the absence of Alcian blue-positive cells in normal esophageal biopsy and its 3D organoid product featuring stratification and squamous-cell differentiation. Representative images of biopsies and 3D organoid products from three independent patients with BE. Scale bar, 50 μm.

FIG. 20 shows the EAC PDO.

FIG. 21 shows the oral normal, preneoplasia and SCC PDOs.

FIG. 22 shows the impact of loss of tumor suppressor gene TP53 in the esophageal epithelium in mice treated with the oral-esophageal carcinogen 4NQO.

FIG. 23 shows that P53 loss in 4 weeks 4NQO treatment promotes preneoplastic cells.

FIG. 24 shows that organoids are useful to test how life-style related risk factor may influence neoplastic changes from individuals with differential genetic factors.

FIG. 25 shows a technique to isolate a single cell-derived organoid clone from culture containing multiple organoids (left) to a smaller number of organoids (middle), and then to a single organoids (right) for clonal analysis.

FIG. 26 shows that tdTomato-positive 3D organoids were generated from primary tumors (primary) and metastatic lesions LN or lung, and tested for tumorigenicity.

FIG. 27 shows that the organoid clones isolated from a primary ESCC tumor or a lung metastatic lesion were used to evaluate their metastatic potential in immunodeficient mice via the tail-vein injection assays.

FIG. 28 shows the differential histopathologic types of esophageal tumors.

FIG. 29 shows that Notch inhibition converts EAC-like structures to ESCC-like structures in 3D organoids.

FIG. 30 shows patient-derived Barrett's Esophagus 3D organoids.

FIG. 31 shows EAC organoids from diagnostic biopsies.

FIG. 32 shows that EAC organoids from individual patients responded differentially to chemo- and targeted therapy.

FIG. 33 provides a summary of esophageal 3D organoid system and its potential applications.

FIGS. 34A-34B show morphological characteristics of organoids derived from normal esophagus and eosinophilic esophagitis (EoE) biopsies. FIG. 34A shows representative images of normal esophageal and EoE organoids. Organoid growth can be monitored via phase contrast microscopy while in culture and histopathological analysis can be conducted on hematoxylin-eosin (H&E)-stained organoids after fixation, paraffin embedding, and sectioning. Expansion of basaloid cell compartment (basal cell hyperplasia) is evident in the EoE organoid. FIG. 34B shows the H&E-stained sections of biopsies from which the organoids were derived.

FIGS. 35A-35B show the modeling reactive epithelium in esophageal organoids. FIG. 35A shows representative images of organoids derived from normal esophageal keratinocytes and treated with eosinophilic esophagitis (EoE)-relevant cytokine IL-13, compared to untreated organoids. Basal cell hyperplasia is evident in IL-13-treated organoid. FIG. 35B shows relative gene expression profiles of EoE-relevant genes SOX2, LOX, and CCL26 (eotaxin) in IL-13-treated organoids, compared to untreated controls. n=3 per group; error bars=standard error of the mean (SEM); asterisk denotes statistical significance.

FIG. 36 shows the workflow of PDO generation and characterization. An esophageal tumor fragment, procured via either endoscopy or surgery, is dissociated and filtered into single cell suspension. Cells are seeded into Matrigel and grown with tumor type-specific organoid culture medium. Resulting PDO are processed for subculture or cryopreservation and subjected to morphological and functional assays coupled with pharmacological drug treatments.

FIGS. 37A-37B show ESCC and EAC PDO morphological characteristics. Representative ESCC (FIG. 37A) and EAC (FIG. 37B) PDO images under phase contrast microscope and histologic characterization of PDO as well as corresponding original primary tumor tissues by Hematoxylin-Eosin (H&E) staining and immunohistochemistry. ESCC PDO comprise poorly differentiated squamous cell carcinoma cells featuring increased cell proliferation (Ki67), stabilization of tumor suppressor TP53 protein, and overexpression of SOX2, an oncogene essential in ESCC. EAC PDO feature high chromatin density along with focal luminal formations reminiscent of glandular structures compatible with adenocarcinoma as corroborated by nuclear expression (arrows) of caudal type homeobox 2 protein (CDX2). Note that TP53 was negative in the representative EAC POD and original primary tumor. Cancer cells within PDO recapitulate those in original tumors. Scale bar, 100 μm.

FIG. 38 shows IC₅₀ curve from drug-treated PDO. EAC PDO size were evaluated by Celigo Imaging Cytometer measuring the mean organoid size following 72 h-exposure to Cisplatin and Paclitaxel at indicated final concentrations. Organoid size was normalized by vehicle-treated control as 100%. IC₅₀ for Cisplatin and Paclitaxel was determined as 7.1 and 2.3 μM (with R squares of 0.5874 and 0.8652), respectively.

FIGS. 39A-39C show the tools used for embedding of paraformaldehyde-fixed PDO. 200-μl pipet tips are modified to make embedding tips and embedding bottom-less barrels (FIG. 39A). A polypropylene 1.7-ml tube rack, covered by a sheet of Parafilm M, is used as a scaffold to place embedding bottom-less barrel where fixed organoids will be cast in along with embedding gel (pre-heated Bacto-agar) (FIG. 39B). To liquefy embedding gel, an aliquot of 5-ml Bacto-agar will be microwaved for 1 min in a 150-ml beaker containing 100-ml water (FIG. 39C).

### DETAILED DESCRIPTION OF THE DISCLOSURE

According to some aspects, the present disclosure provides a method for stratifying the risk of developing a tumor in a subject, comprising: obtaining a biological sample from the subject; generating a three-dimensional (3D) organoid system from the biological sample; and detecting one or more dysplastic 3D structures.

In some embodiments, the subject has oral preneoplasia. In some embodiments, the tumor is an oral squamous cell carcinoma (OSCC). In some embodiments, the method further comprises the steps of: determining the aldehyde dehydrogenase (Aldh)-2 genotype of the subject using the 3D organoid system; identifying the subject as having high risk of developing the cancer, if the Aldh2 genotype is Aldh2^{E487K}; and initiating a therapeutic protocol that prevents the progression of the tumor.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. In some embodiments, the subject is a human.

As used herein, a “biological sample” is a sample obtained from a subject. Biological samples include all clinical samples useful for detection of disease or genetic information (for example, cancer or specific genotype) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In some embodiments, the biological sample is originating in oral, pharyngeal or esophageal mucosa.

According to some aspects, the present disclosure provides a method for treating or ameliorating the effects of a tumor in a subject, comprising: obtaining a biological sample from the subject; generating a three-dimensional (3D) organoid system from the biological sample; determining the aldehyde dehydrogenase (Aldh)-2 genotype of the subject using the 3D organoid system; and administering to the subject with an effective amount of a chemotherapy agent, if the Aldh2 genotype is Aldh2^{E487K}.

In some embodiments, the tumor is an oral squamous cell carcinoma (OSCC). In some embodiments, the biological sample is an originating in oral, pharyngeal or esophageal mucosa.

As used herein, a “chemotherapy agent” or “chemotherapeutic agent” is any chemical agent with therapeutic usefulness in the treatment of diseases characterized by abnormal cell growth. For example, chemotherapy agents are useful for the treatment of cancer, including but not limited to squamous cell carcinoma, esophageal cancer and adenocarcinoma. Particular examples of chemotherapeutic agents that can be used include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. In some embodiments, the chemotherapy agent is selected from the group consisting of actinomycin all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil (5FU), gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and combinations thereof. In some embodiments, the chemotherapy agent is selected from cisplatin, fluorouracil (5FU), and combinations thereof.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

According to some aspects, the present disclosure provides a method for improving the efficacy of chemotherapy in a subject with a tumor, comprising obtaining a biological sample from the subject; generating a three-dimensional (3D) organoid system from the biological sample; determining the aldehyde dehydrogenase (Aldh)-2 genotype of the subject using the 3D organoid system; and co-administering to the subject with an effective amount of a chemotherapy agent and an effective amount of an agent that inhibits Aldh2, if the Aldh2 genotype is Aldh2^{E487K}.

In some embodiments, the agent that inhibits Aldh2 is selected from the group consisting of ampal, benomyl, citral, chloral hydrate, chlorpropamide, coprine, cyanamide, daidzin, CVT-10216, DEAB, DPAB, disulfiram, gossypol, kynurenine tryptophan metabolites, molinate, nitroglycerin, pargyline, and combinations thereof. In some embodiments, the agent that inhibits Aldh2 is disulfiram. In some embodiments, the agent that inhibits Aldh2 is administered to the subject before, concurrent with or after the administration of the chemotherapy agent.

According to some aspects, the present disclosure provides a method of treating or ameliorating the effects of an oral tumor in a subject comprising the steps of: detecting the presence of the aldehyde dehydrogenase 2 (Aldh2) single nucleotide polymorphism (SNP) Aldh2^{E487K} in the subject; and administering a chemotherapy agent if Aldh2^{E487K} is detected.

According to some aspects, the present disclosure provides a method of treating or ameliorating the effects of an oral tumor comprising the steps of: detecting the presence of Aldh2^{E487K}; and administering an Aldh2 inhibitor and a chemotherapy agent.

According to some aspects, the present disclosure provides a method of screening for efficacy of a chemotherapy agent against a tumor comprising the steps of: obtaining a biological sample from a subject; generating a three-dimensional (3D) organoid system from the biological sample; and contacting one or more cells of the 3D organoid system with one or more chemotherapy agents to identify efficacy of the chemotherapy agent.

In some embodiments, the method further comprises the step of detecting the presence of the aldehyde dehydrogenase 2 (Aldh2) single nucleotide polymorphism (SNP) Aldh2^{E487K}.

As used herein, an “effective amount” or “therapeutically effective amount” of an agent (e.g., a chemotherapy agent) is an amount of such an agent that is sufficient to affect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of an agent according to the present disclosure will be that amount of the agent, which is the lowest dose effective to produce the desired effect with no or minimal side effects. The effective dose of an agent according to the present disclosure may be administered as two, three, four, five, six or more sub-doses, administered separately at appropriate intervals throughout the day

The following examples are provided to further illustrate certain aspects of the present disclosure. These examples are illustrative only and are not intended to limit the scope of the disclosure in any way.

### EXAMPLES

### Example 1

### 3D Organoids as a Novel Platform for Prevention and Therapy of OSCC

A three-dimensional (3D) organoid system has been developed (Kijima et al. 2019; Whelan et al. 2018; Kasagi et al. 2018; Natsuizaka et al. 2017), which is a cell culture-based platform with tissue-like architecture grown in a mount of basement membrane extract (Matrigel) with media containing niche factors (Vermorken et al. 2008; Kijima et al. 2019; Whelan et al. 2018; Kasagi et al. 2018). The morphological and functional characteristics of OP and OSCC can be recapitulated in 3D organoids generated from single-cell suspensions isolated from original tissues. The 3D organoids can serve as an excellent tool to explore genes and pathways altered during cancer progression, gene-drug association and personalized therapy design.

The foremost etiologic factors for OSCC include tobacco and alcohol. Both tobacco smoke and alcohol metabolites contain acetaldehyde, a major human carcinogen. Acetaldehyde and other toxic aldehydes are broken down via mitochondrial aldehyde dehydrogenase (ALDH)-2. ALDH2 dysfunction increases cancer risk in individuals with single nucleotide polymorphism (SNP) referred to as ALDH2*2, carried by >8% of the entire world population and 30-40% of East Asians (Chinese, Korean and Japanese). Producing mutant Aldh2 protein (Aldh^{E487K}), this SNP is strongly associated with OSCC (Salaspuro and Salaspuro, 2004).

Studies have indicated that Aldh2 dysfunction delays aldehyde clearance to cause mitochondrial dysfunction and DNA damage via oxidative stress (Tanaka et al. 2016). Aldh2 dysfunction may increase OSCC tumor initiating cells (or cancer stem cells) defined by high CD44 expression (CD44H cells) through activation of cellular antioxidants such as SOD2 (Kinugasa et al. 2015) and autophagy that removes dysfunctional mitochondria (Whelan et al. 2017).

In 3D organoids, disease progression and therapy resistance are associated with an increase in CD44H cells and the Aldh2*2 status. Interestingly, cancer cells with Aldh2*2 responded better to chemotherapeutic agents than those with wild-type Aldh2 (aka Aldh2*1). Thus, combined chemotherapy and either pharmacological Aldh2 inhibition or enhancement of oxidative stress may benefit patients carrying wild-type Aldh2*1.

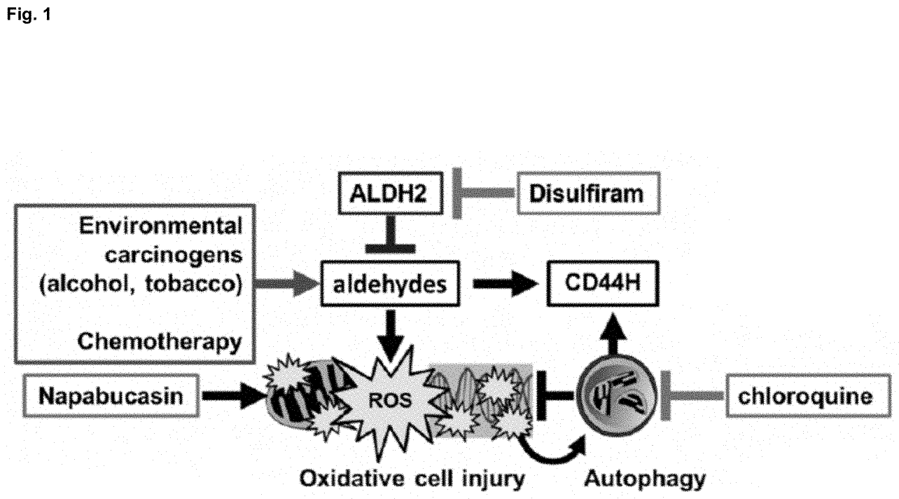

This Example was to build and characterize a 3D organoid library representing OP and OSCC from mice and patients with ALDH2*2 or ALDH2*1. The central hypothesis is that ALDH2*2 promotes OSCC progression via CD44H cells that may be targeted for therapy by enhancing oxidative cell injury (FIG. 1). Experiments were carried out: 1) to unravel the tumor suppressor role of ALDH2 in OSCC progression, and 2) to evaluate pharmacological stimulation of oxidative stress to target CD44H OSCC tumor initiating cells.

The experiments were designed to utilize the highly innovative 3D organoid system coupled with established genetically engineered mouse models and patients' biopsies with pharmacological modulations with a goal to facilitate development and validation of 3D organoids as a novel platform for prevention and therapy of OSCC in the setting of precision medicine. Additionally, this comprehensive platform can fundamentally advance our understanding as to how Aldh2 dysfunction may foster tumor initiating cells (i.e. CD44H cells) in concert with environment carcinogens and that how Aldh2 SNP may influence differential therapy response in patients.

### 3D Organoids are Used for Ex Vivo Functional Analyses of Oral Preneoplastic (OP) and OSCC Cells

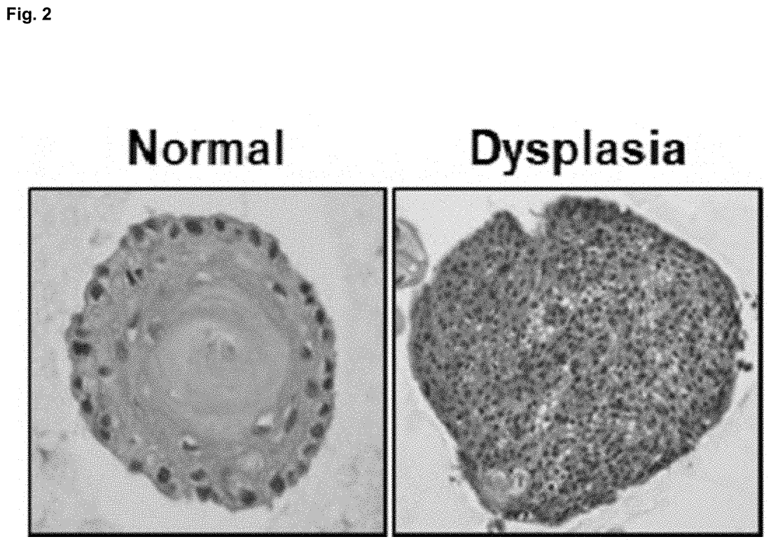

The 3D organoid system was developed (Kijima et al. 2019; Whelan et al. 2018; Kasagi et al. 2018; Natsuizaka et al. 2017), recapitulating the morphology and physiology of the originating oral, pharyngeal and esophageal mucosa, all continuous and sharing the stratified squamous epithelia. Utilizing IRB-approved biopsies, 3D organoids were generated from patients with OP or OSCC and normal oral mucosa (FIG. 2). Normal 3D organoids exhibit a differentiation gradient with keratinization, whereas OP and OSCC 3D organoids display progressively atypism represented by nuclear hyperchromasia, cellular crowding, increased mitotic activity and other cellular abnormalities.

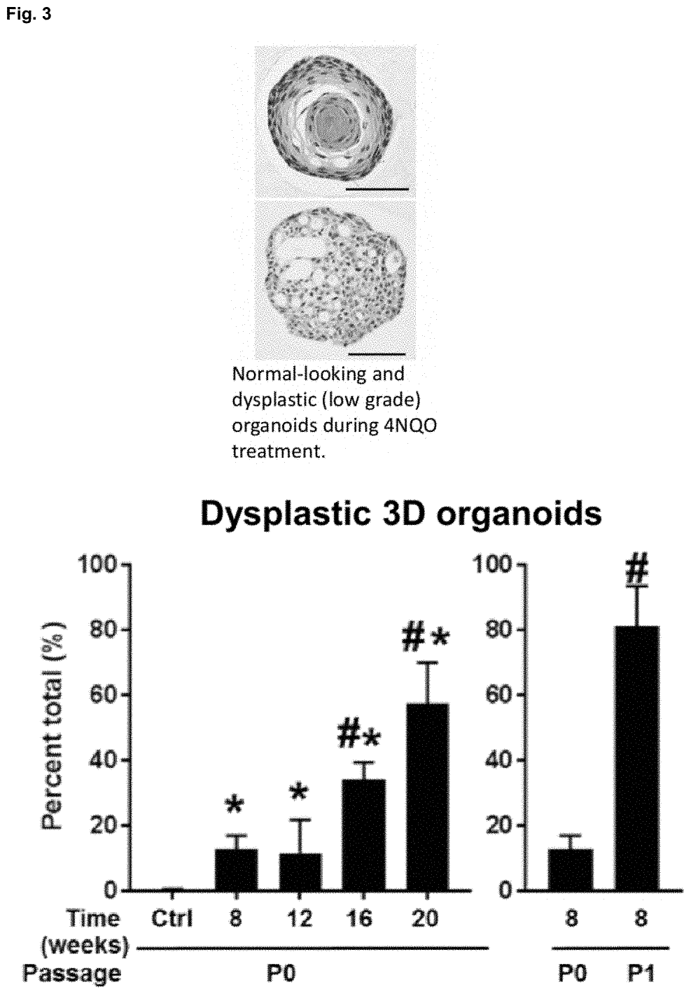

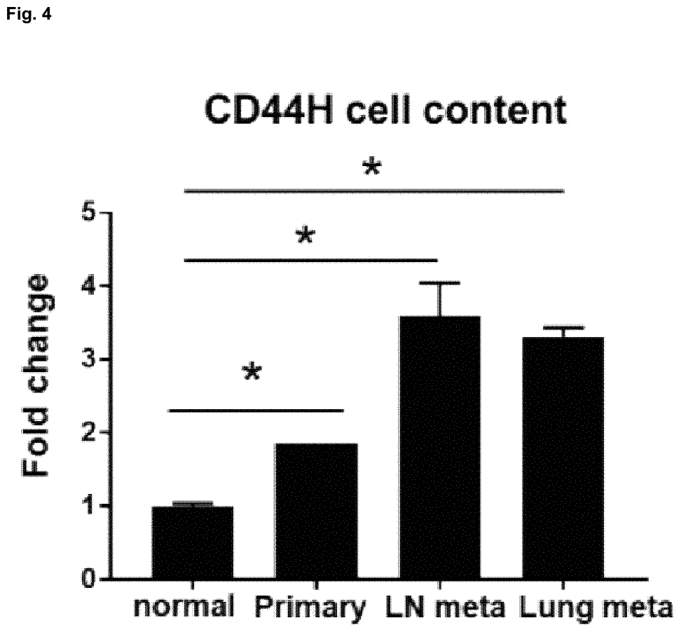

3D organoid assays are highly sensitive to detect preneoplastic cells. This was validated in 3D organoids generated from mice treated with 4-nitroquinoline 1-oxide (4NQO), a potent oral-esophageal carcinogen that induces DNA lesions similar to those induced by tobacco smoke in human. In the 4NQO model, neither visible tumors nor histologic neoplastic lesions emerge until 20 weeks from the start of 4NQO treatment (Natsuizaka et al. 2017; Tang et al. 2004). In 3D organoids, dysplastic 3D structures (FIG. 3) were detected as early as 8 weeks from the start of 4NQO treatment and that dysplastic 3D structures propagated rapidly when passaged (FIG. 3), indicating that 4NQO-induced dysplastic cells may have enhanced capability of self-renewal and proliferation compared to 4NQO-untreated normal epithelial cells. Moreover, 3D organoids from 4NQO-induced metastatic tumors contained more CD44H cells than those from primary tumors (FIG. 4). These self-renewable and single cell-derived 3D organoids serve as a novel platform to compare species, organ and disease stage specific differences ex vivo that may be observed in mice and patient derived organoids.

### ALDH2 is a Limiting Factor of OP Progression and May be Targeted to Improve OSCC Chemotherapy

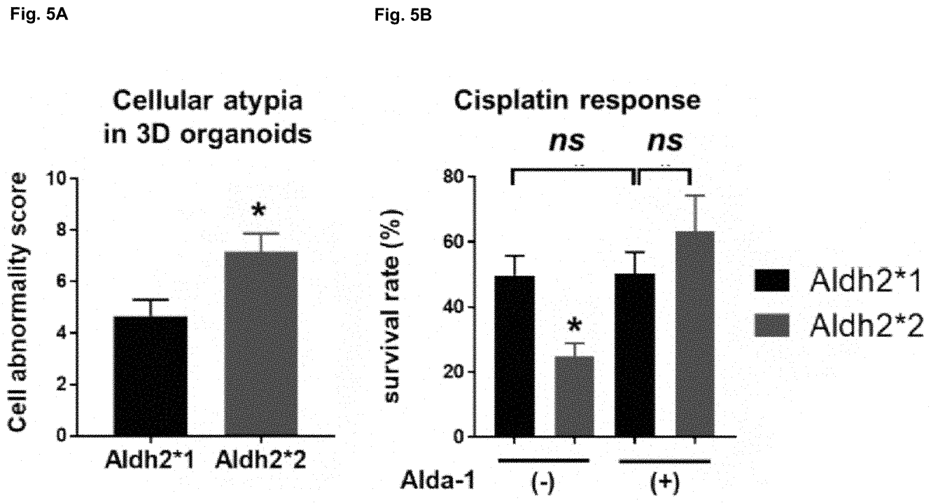

Aldh2*2 is a common single nucleotide polymorphism (SNP) strongly associated with OSCC. To evaluate the role of Aldh2*2 in 4NQO-induced neoplastic cells in 3D organoids, Aldh2*2 mice expressing mutant Aldh2 protein (Aldh2^{E487K}) were compared with wild-type Aldh2*1 (control) mice. 3D organoids were analyzed from tumors detected in mice sacrificed at 26 weeks from the start of 4NQO treatment. Histopathologic analyses of resulting 3D organoids revealed that cancer cells with Aldh2*2 displayed greater cellular atypia than those with Aldh2*1 (FIG. 5A), suggesting that Aldh2 mutation may promote malignant transformation. Interestingly, Aldh2*2 (Aldh2 mutant) tumor organoids appeared to respond to chemotherapy agents (cisplatin and 5FU) better than Aldh2*1 (Aldh2 wild-type) tumor organoids (FIG. 5B and data not shown) where cisplatin-mediated cytotoxicity was reversed by Alda1 (pharmacological Aldh2 activator) in Aldh2*2 tumor 3D organoids, indicating a direct involvement of the Aldh2 activity in chemotherapy response. Thus, individuals with Aldh2*2 SNP may respond to chemotherapy better despite its strong association with a risk of OSCC tumor development. It was hypothesized that pharmacological Aldh2 inhibition by Disulfiram (Antabuse, used a treatment of chronic alcoholism) augments the chemotherapy effects in OSCC in individuals with Aldh2*1. Thus, patient-derived 3D organoids may serve as a platform for new translational applications in personalized medicine for therapy of OSCC and potentially other squamous cell carcinomas.

### Methods

Tissue processing: murine and IRB-approved patient OP and OSCC biopsies will be analyzed (Kijima et al. 2019; Giroux et al. 2017; Kalabis et al. 2012). For 3D organoids and DNA, RNA and flow cytometry analyses, a single cell suspension will be prepared by enzymatic dissociation. Enzyme units, incubation time and temperature pre-optimized will be used to minimize proteolytic loss of cell surface epitopes (Kasagi et al. 2018; Natsuizaka et al. 2017; Whelan et al. 2017). Dissociated cells will be forced through 70 μm strainer to yield >0.3×10⁶ viable cells per specimen (Kasagi et al. 2018; Natsuizaka et al. 2017; Whelan et al. 2017). >80% cells remain viable for hours at 4° C. in PBS containing 1% bovine serum albumin and antibodies for flow cytometry for cell surface markers such as CD44. 3D organoids and original tissue samples will be fixed in formalin and paraffin-embedded for morphological studies.

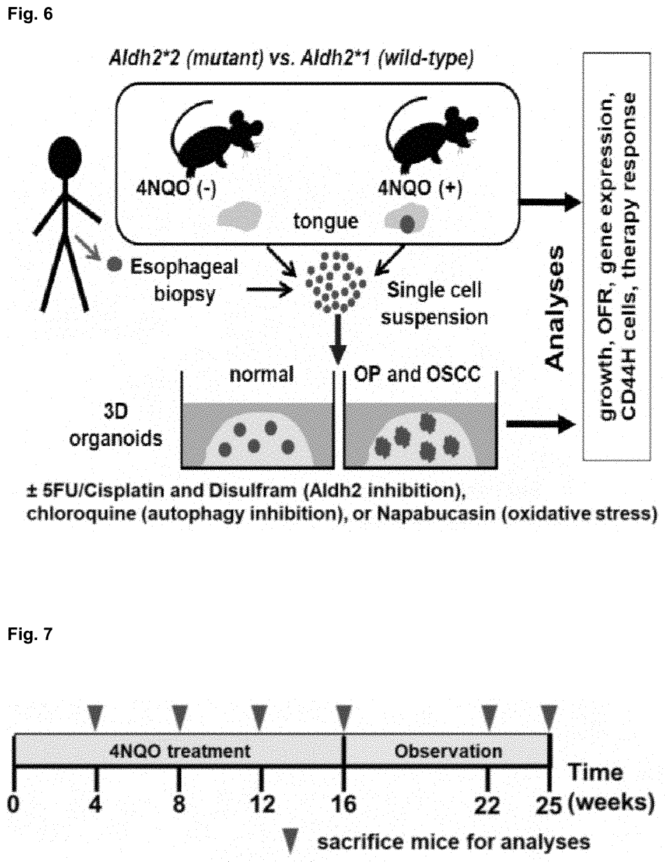

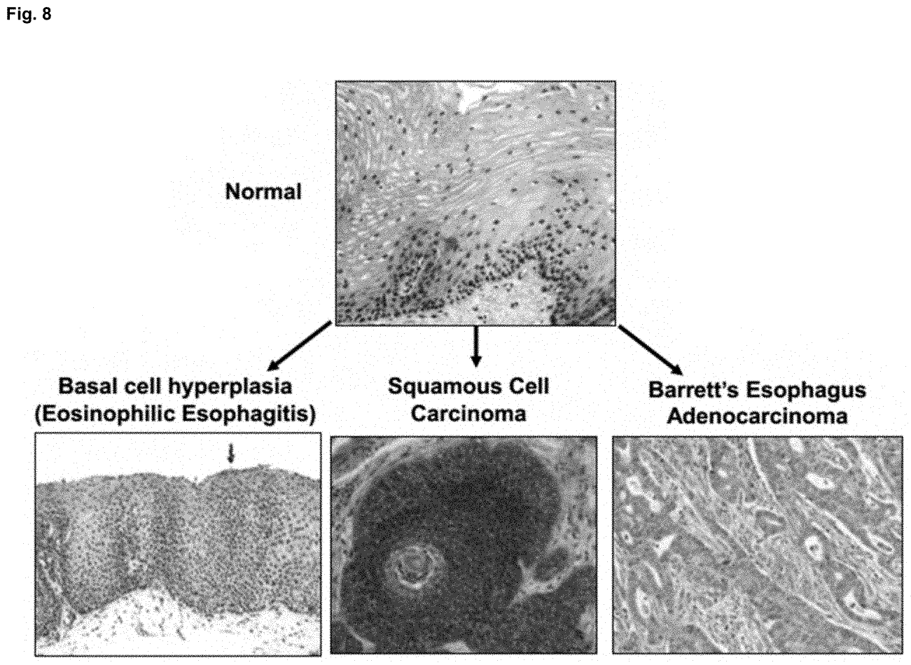

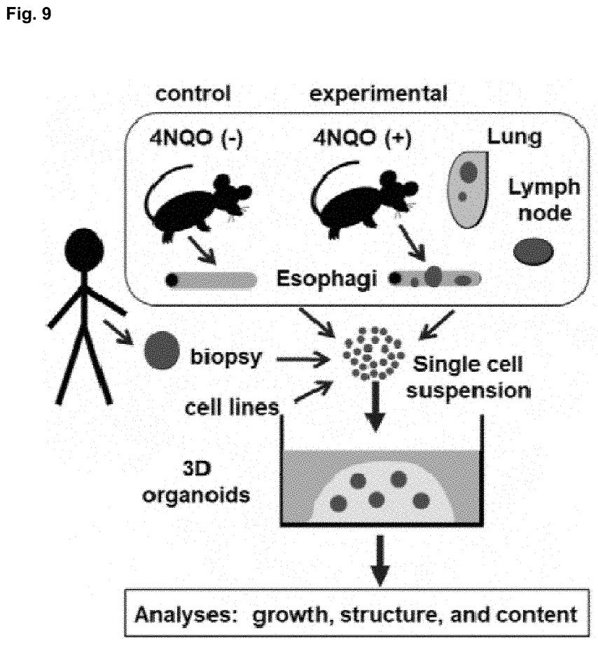

Oral 3D organoids (FIG. 6): 3D organoids will be generated according to the published protocols (Kasagi et al. 2018; Natsuizaka et al. 2017; Whelan et al. 2017). In 24-well plates, 2,000 cells are seeded in Matrigel (50 μl/well). Organoids grow within 7-10 days to form 50-100 μm spherical structures (100% success, n=34 for human normal biopsies) (Kasagi et al. 2018). For quality control purposes, an OSCC cell line will be used (Kijima et al. 2019). Time-lapse analysis of growing organoids will be done under light microscopy to determine their average size and morphology. organoid formation rate (OFR; the percentage of the number of organoids formed at day 10 per total number of cells seeded at day 0) will be determined. Organoids will then be processed for DNA, RNA and flow cytometry, and histology. Additionally, organoids will be dissociated and passaged to determine self-renewal of organoid initiating cells within the primary organoids. Treatment with drugs will be initiated at day 7 for 3 days to assess effects upon organization of established structures, cell viability and CD44H (tumor initiating) cell content.

DNA, RNA and Flow cytometry: Aldh2 genotype, CD44 isoforms (Tanaka et al. 2016; Whelan et al. 2017), and CD44H cells (Natsuizaka et al. 2017; Whelan et al. 2017; Whelan et al. 2017) will be determined in 3D organoids. RNA from 3D organoids will be used for RNA-seq analyses.

Histology analyses: cellular atypia, Ki67 (proliferation), p53 and p16 (tumor suppressors), EGFR and cyclin D1 (oncogenes), and vH2AX (oxidative stress) will be scored in 3D organoids and original tissues by histology and immunohistochemistry. The labeling index will be determined by counting at least 600 cells per section.

Patient-derived 3D organoids: IRB-approved biopsies from therapy-naive OP and OSCC patients and adjacent normal mucosa from the same patients (when available) will be procured. It is planned to analyze 6 OSCC and 12 OP adult patients (9 African Americans, 6 Asians, 6 Caucasians) while pediatric OP/OSCC patients are rare. Growth kinetics, Aldh2 genotype, gene expression (RNA-seq), tumor initiating cells characterized by CD44H cells, and morphology will be analyzed. Resulting neoplastic organoids will be injected into immunodeficient mice (two injection sites/mouse, 4 mice/patient) to determine tumorigenicity and also perform tail-vein injection assays to evaluate lung metastasis. Data will be interpreted in light of the histopathology data of original tissues as well as the ALDH2 genotype determined in each human subject.

Murine 3D organoids: Aldh2*2 (mutant) and Aldh2*1 (wild-type) mice (Zambelli et al. 2014) will be treated with 4NQO, a potent oral-esophageal carcinogen that induces DNA lesions similar to those found in human. 3 mice at each time point will be sacrificed (FIG. 7) per group (genotype) as a biological replicate to insure >80% power based on our murine 4NQO and 3D organoid studies (Natsuizaka et al. 2017). Both male and female will be used as they show no significant difference in tumor formation (Natsuizaka et al. 2017). Mice at age of 2-3 month will receive 100 μg/ml 4NQO or 2% propylene glycol (control) in drinking water containing 10 g/L glucose, starting at the age of 2-3 month, for 16 weeks and followed by an observation period for 9 weeks (Natsuizaka et al. 2017). >95% of Aldh2*2 mice are expected to display OP and OSCC (and similar lesions in the esophagus) at 22 and 25 week time points, respectively (Natsuizaka et al. 2017). 4NQO-untreated Aldh2*2 and Aldh2*1 mice (control) will also be sacrificed. OP and OSCC cells will be evaluated in 3D organoids.

Drug treatments: In 3D organoids, half-maximal inhibitory concentrations (IC₅₀) will be determined for 5-fluorouracif (5FU) and cisplatin (CDDP), chemotherapeutic agents used for standard of care of OSCC as described previously (Kijima et al. 2019). 3D orgnaoids from 6 patients and mice (22-25 week time points) will be included. 3D organoids will be treated for 3 days (6 wells per drug for each concentration). They will be treated concurrently with or without Disulfiram (a clinically used ALDH2 inhibitor), Chloroquine (autophagy inhibitor, a clinically used anti-malaria agent), and Napabucasin (Boston Biomedical), all expected to augment oxidative cell injury (FIG. 1). CellTiter-Glo® 3D cell viability assay (Promega) will be used. Additionally, CD44H cells and oxidative cell injury/DNA damage will be evaluated in 3D organoids surviving chemotherapy.

### Example 2

### 3D Modeling of Esophageal Diseases and Precision Medicine

### Tissue Processing, Cell Isolation and Esophageal 3D Organoids

3D organoids will be generated from IRB-approved patients' endoscopic biopsies, surgically resected tissues and murine tissues (e.g. tumor-bearing esophagus, metastatic lung tumors, metastatic lymph nodes) according to our published protocols (Nakagawa et al. 2020; Karakasheva et al. 2020). Single cell suspensions with >80% viability will be prepared (Kasagi et al. 2018; Whelan et al. 2017; Natsuizaka et al. 2017). FIG. 9 illustrates an example from mice bearing tumors induced by 4NQO, an oral-esophageal carcinogen. Similar experiments can be done utilizing a variety of mouse models of non-neoplastic diseases such as Barrett's esophagus (BE) or eosinophilic esophagitis (EoE). To generate 3D organoids, 2000 cells are seeded in Matrigel (50 μl/well). Under optimized conditions, organoids grow within 7-10 days to form 50-100 μm spherical structures (e.g. 100% success, n=34 for human normal biopsies) (Kasagi et al. 2018). For quality control purposes, a normal human esophageal cell line EPC2 will be used (Harada et al. 2003). Time-lapse analysis of growing organoids will be done under light microscopy to determine their average size and morphological composition. Organoid formation rate (OFR; the percentage of the number of organoids formed at day 10 per total number of cells seeded at day 0) will be determined. Resulting 3D organoids will then be processed for histology or flow cytometry. Additionally, organoids will be dissociated and passaged to form secondary organoids to determine self-renewal of organoid initiating cells within the primary organoids and for analyses described in each aim. Treatment with drugs (e.g. CQ) will be initiated at day 0 to evaluate effects upon organoid formation and growth, or at day 7 to assess effects upon organization of established structures.

### Loss of Notch1 May Foster a Basal Cell Subpopulation to Continue Epithelial Renewal in Basal Cell Hyperplasia Modeled in 3D Esophageal Organoids

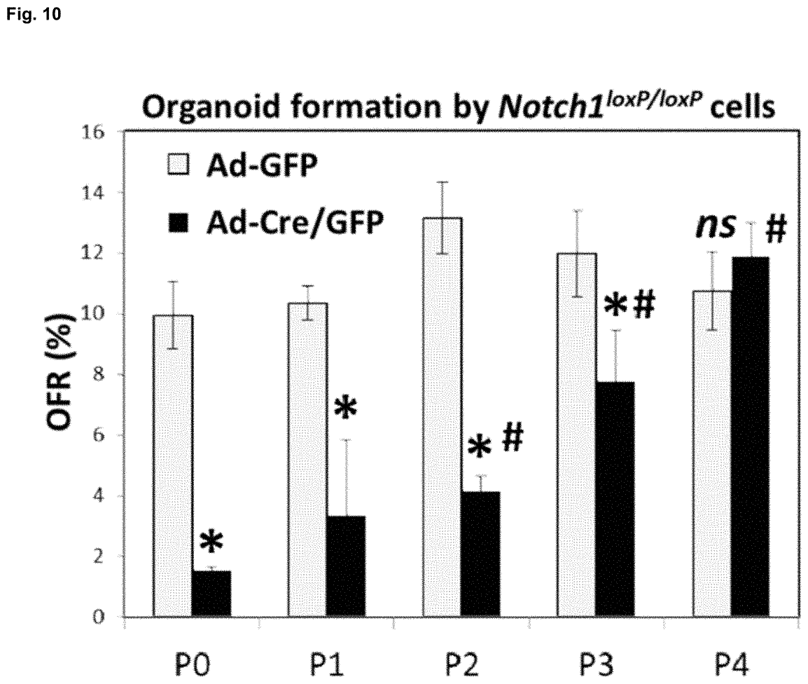

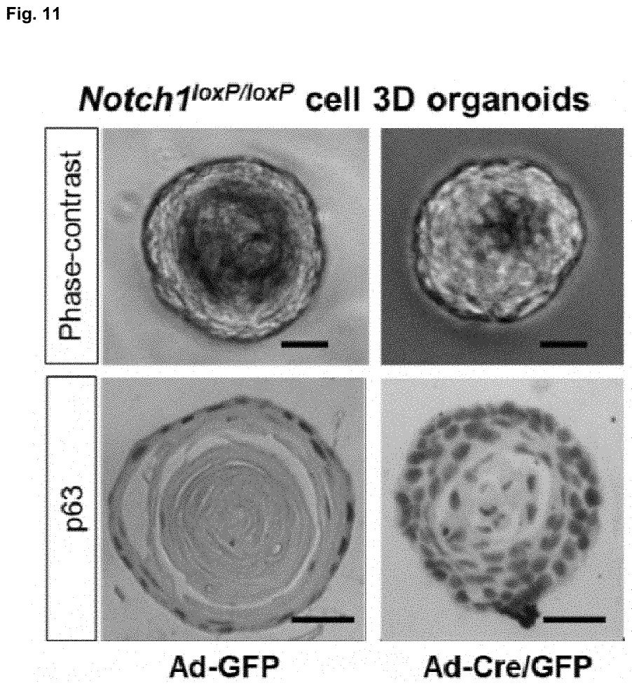

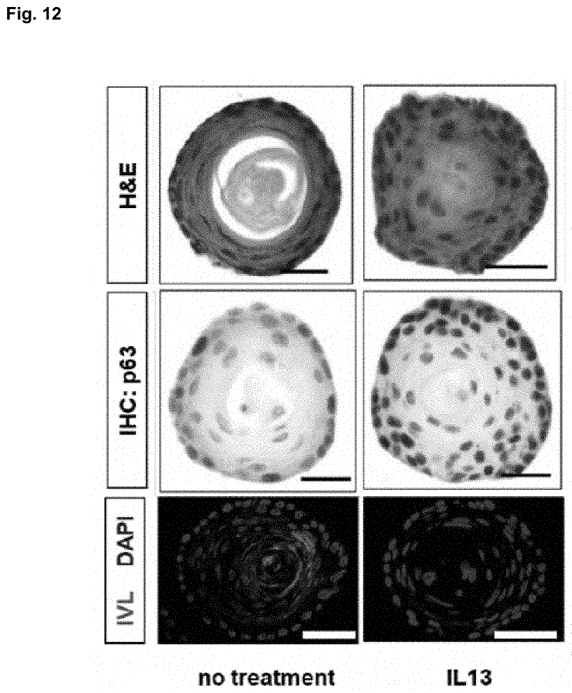

Basal cell hyperplasia (BCH) is a common histopathological feature of esophageal diseases such as eosinophilic esophagitis, radiation-induced esophagitis and GERD. BCH is induced by injury or inflammation and involves an expansion of basal epithelial cells (>20% of epithelial height) with limited formation of intercellular bridges (desmosomes/tight junctions), a hallmark of squamous-cell differentiation in the esophagus. Notch maintains esophageal epithelial integrity by regulating squamous-cell differentiation (Ohashi et al. 2010). Notch signaling is downregulated in BCH modeled 3D organoids (Kasagi et al. 2018). Notch1loxP/loxP mice was utilized to evaluate how loss of Notch1 (a prototype of Notch) may influence epithelial formation and renewal by single cell-derived 3D organoid formation assays. In a cell suspension prepared from an esophageal epithelial sheet, adenoviral Cre (Ad-Cre/GFP) was transduced to delete Notch1 at the onset of 3D organoid culture. Loss of Notch1 significantly suppresses organoid formation at initial primary culture (P0), compared to control cells transduced with a control virus (Ad-GFP) expressing green fluorescent protein (GFP) only (FIG. 10). This finding suggests that the majority of organoid-initiating basal cells require Notch1 to form the epithelium of 3D organoids while a minority of co-existing cells initiate organoids to form the epithelium in a Notch1-independent manner. Notch1-deleted (Notch1−/−) and control (Notch1loxP/loxP) organoids were recovered at day 7 and dissociated into single cell suspensions. By FACS, GFP-positive cells were purified to validate Notch1 loss by qPCR (data not shown). The remaining cells were passaged into subculture. Interestingly, organoid formation by Notch1-deleted cells was increased from passage to passage while organoid formation by control cells did not change over passages, suggesting that Notch1 loss may permit enrichment of basal cells with an organoid initiating capability (FIG. 10). In agreement with such a premise, Notch1−/− 3D organoids had a structure compatible with BCH as evidenced by expansion of p63+ basal cells with diminished differentiation (FIG. 11). By contrast, control organoids showed an exquisite differentiation gradient (FIG. 11). These findings suggest that the esophagus may contain a subset of basal cells that can self-renew and foster BCH, negating Notch-dependent squamous cell-differentiation.

### Modeling Reactive Epithelial Changes and Genetic and Pharmacological Modifications

The mucosa of the upper aerodigestive tract (e.g. oral cavity, pharynx, esophagus) comprises stratified squamous epithelium in which epithelial cells (keratinocytes) exhibit a proliferation-differentiation gradient and provide a barrier against the chemical and biological milieu of luminal contents. Disruption of this differentiation gradient or barrier function is linked to multiple human pathologies such as eosinophilic esophagitis, Gastroesophageal reflux disease (GERD) and intestinal metaplasia (Barrett's esophagus or BE) feature aberrant epithelial cell proliferation and differentiation. We have developed the 3D organoid system (Nakagawa et al. 2020; Kasagi et al. 2018; Whelan et al. 2017; Whelan et al. 2018) for ex vivo functional analyses of epithelial renewal and differentiation. Esophageal 3D organoids recapitulate the morphology and physiology of originating tissues (Whelan et al. 2018). Utilizing IRB-approved human endoscopic biopsies, a patient-derived 3D organoid library was built representing normal esophageal mucosa (n=8), active EoE (n=9), EoE in remission (n=12), and GERD (n=3) (Kasagi et al. 2018). These organoids have been cryopreserved at early passages (P1-P2). Normal esophageal organoids show a Notch-dependent differentiation gradient (Kasagi et al. 2018). Multiple cytokines induce BCH-like changes in human and murine esophageal 3D organoids (Kasagi et al. 2018; Whelan et al. 2017) (FIG. 12).

### 3D Organoids are Used for Ex Vivo Functional Analyses of Normal, Premalignant and Metastatic Oral/Esophageal Squamous Cell Carcinoma (SCC) Cells

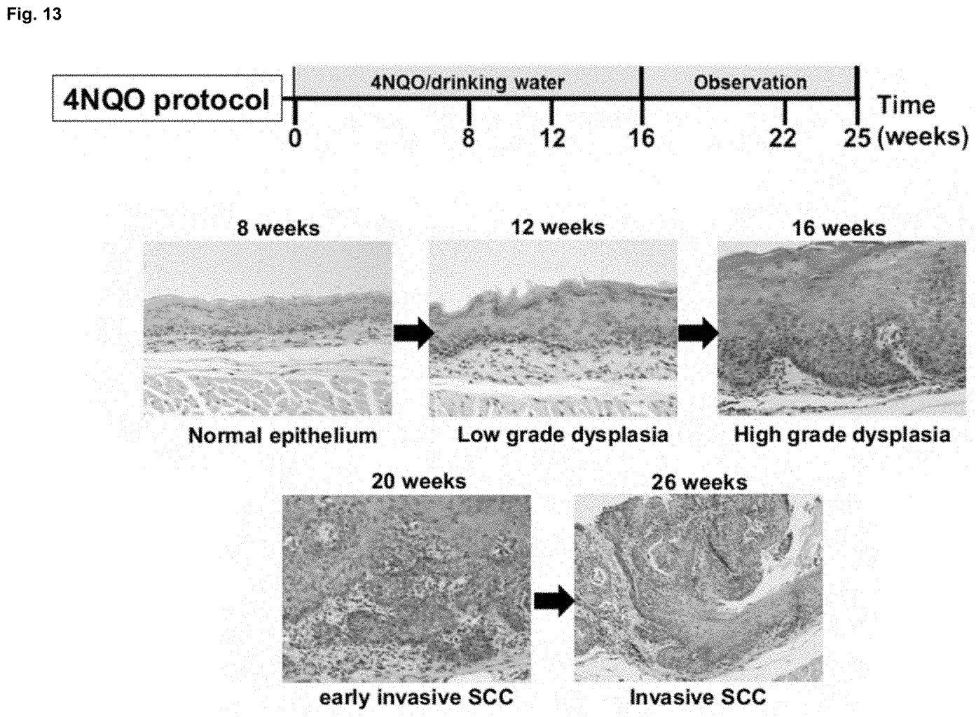

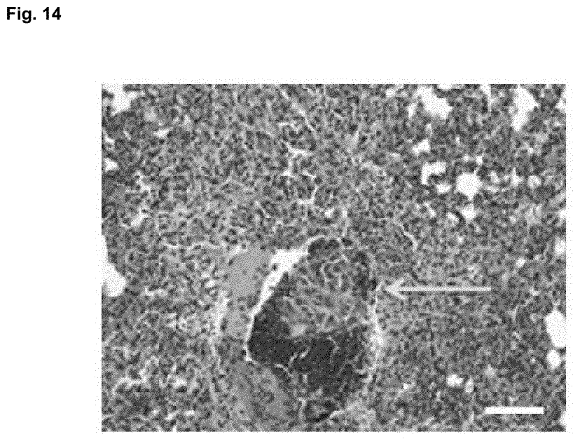

The oral and esophageal carcinogen 4-nitroquinoline 1-oxide (4NQO) induces premalignant and cancer lesions (FIG. 13) in mice at a high frequency (˜100%) within a predictable time frame (4-8 weeks after 4NQO exposure)(Natsuizaka et al. 2017; Tang et al. 2004; Long et al. 2015). This was the first time that 4NQO-induced metastatic lesions were documented (FIG. 14). Amongst the cell surface markers defining a distinct subset of cancer cells is CD44. As a major receptor for hyaluronic acid, CD44 has a role in cancer cell invasion, metastasis and drug resistance by mediating crosstalk between cancer cells and the tumor microenvironment (Toole and Slomiany, 2008; Twarock et al. 2010; Takayama et al. 2003; Zoller, 2011). A high CD44 (CD44H) level has been linked to tumor initiating capability in esophageal squamous cell carcinoma (ESCC) and other SCCs (Zoller, 2011; Al-Hajj et al. 2003; Prince et al. 2007; Zhao et al. 2011; Kijima et al. 2019). The origin of CD44H cells remains elusive. CD44H cells are found in the invasive tumor front (Natsuizaka et al. 2017), a specialized niche that fosters tumor progression (Liotta and Kohn, 2001; Christofori, 2006; Wels et al. 2008). Both SCC and premalignant cells display pleomorphism including spindle-shaped morphology (Gal et al. 1987; Gustafsson et al. 2005) compatible with epithelial-mesenchymal transition (EMT) which is defined by loss of epithelial characteristics (e.g. cell-cell adhesion, expression of E-cadherin and EpCAM) and gain of mesenchymal characteristics (e.g. increased motility and N-cadherin expression). EMT is associated with cancer cell invasion, metastasis, chemotherapy resistance and poor prognosis in ESCC and other SCCs (Basu et al. 2010; Uchikado et al. 2005; Usami et al. 2008). To document EMT during ESCC development and progression in mice, cell-lineage traceable transgenic mice were developed to express Cre recombinase in oral and esophageal basal keratinocytes in an either constitutive (L2Cre) (Stairs et al. 2011) or tamoxifen-inducible (K5CreERT2) (Natsuizaka et al. 2017) manner. These mice carry the Rosa26 locus with knocked-in YFP or tdTomato as a reporter under the loxP-stop-loxP sequence. Thus, all oral and esophageal epithelial cells that underwent Cre-mediated recombination will be permanently marked with fluorescent protein YFP or tdTomato. Both L2Cre and K5CreERT2strains show near 100% recombination efficiency (i.e. YFP labeling) (Natsuizaka et al. 2017; Stairs et al. 2011).

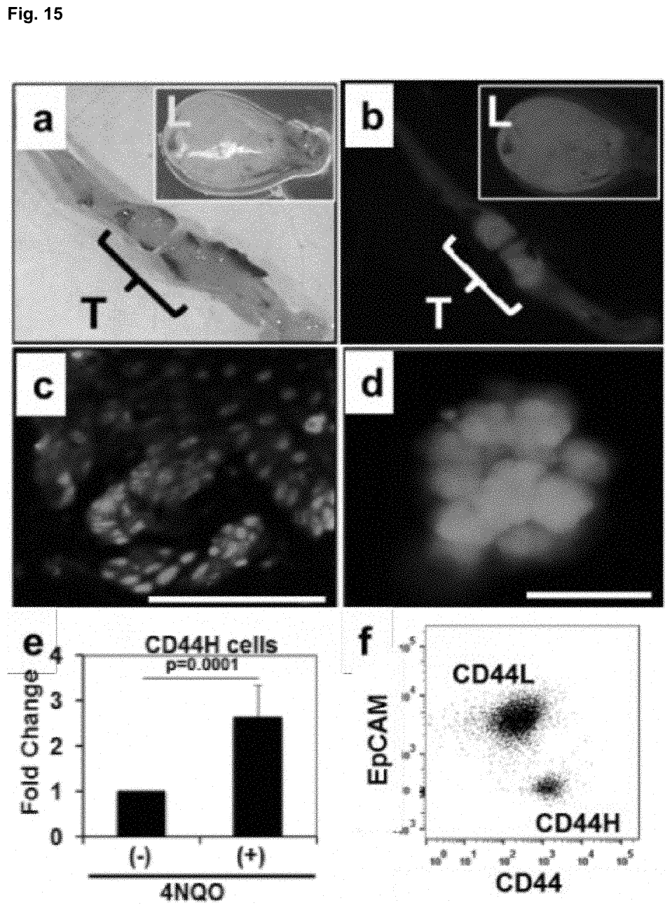

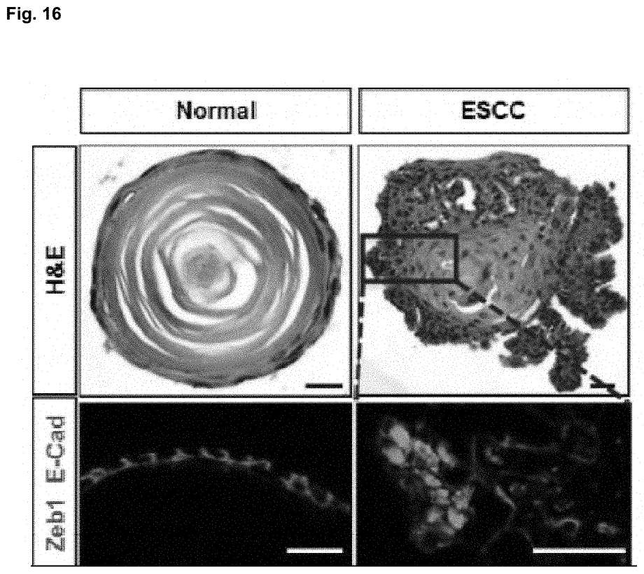

With fluorescent dissecting microscope, 4NQO-induced esophageal squamous cell carcinoma (ESCC) tumors with YFP expression were detected in L2Cre; R26YFPlsl/lsl mice. Immunofluorescence (IF) detected microscopically YFP-positive invasive lesions (FIG. 15, panels a-c), indicating epithelial cell of origin even if YFP+ cells may lose epithelial characteristics via EMT. YFP+ ESCC cells were isolated by fluorescence-activated cell sorting (FACS) to form 3D organoids grown ex vivo to display highly irregular neoplastic structures (FIG. 15, panel d). Flow cytometry shows an increase in YFP+ cells with high CD44 expression (CD44H) and compatible with EMT (i.e. EpCAM-negative) within 4NQO-induced tumors as compared to normal esophageal mucosa from untreated controls (FIG. 15, panels e and f). Organoids from normal and 4NQO-induced dysplastic and ESCC lesions in mice with a cell-lineage traceable genetic modification were analyzed. These single cell-derived 3D organoids serve as a novel platform to compare species and disease stage-specific differences that may be observed in mice, human cell lines and patient-derived cells. Organoids from 4NQO-untreated (normal) control mice exhibit a differentiation gradient, whereas tumor-derived organoids (neoplastic) display irregular structures with increased cellularity, atypia, and diminished differentiation (FIG. 16) (Natsuizaka et al. 2017). EMT was robust in tumor organoids (Natsuizaka et al. 2017). Organoids can be manipulated ex vivo pharmacologically or genetically (Natsuizaka et al. 2017; Kijima et al. 2019; Kasagi et al. 2018), requiring a minimal number of mice (n=2-4) per genotype and experiment with multiple technical replicates and >80% statistical power. Utilizing IRB-approved endoscopic biopsies, patient-derived normal and ESCC 3D organoids were generated (Kijima et al. 2019; Kasagi et al. 2018), which will serve as a platform to test molecularly-targeted therapeutics to reduce CD44H cells and that is more readily translatable (personalized medicine).

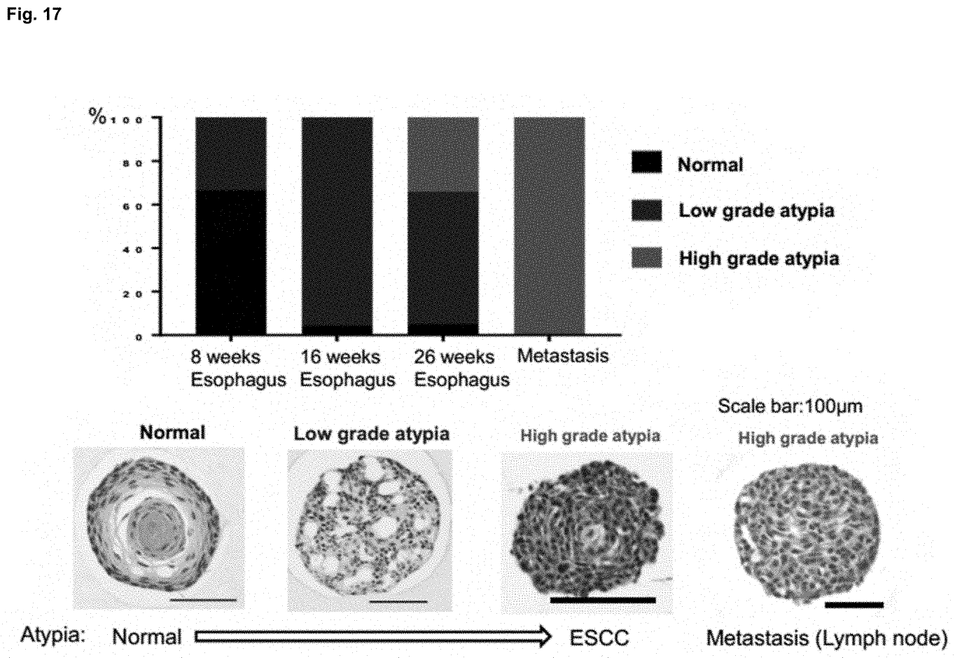

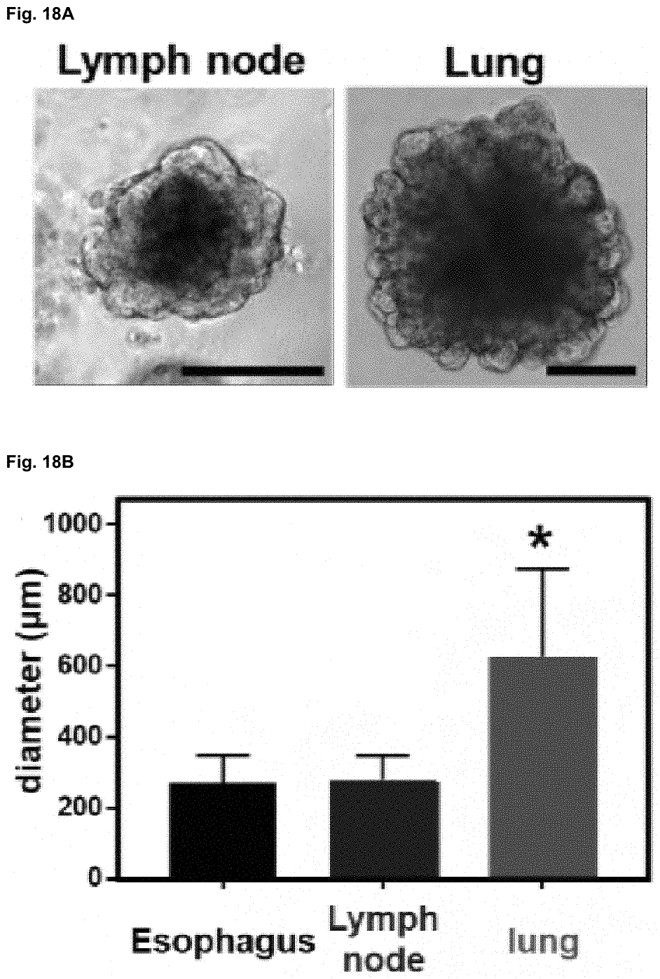

Analyzing live premalignant and ESCC cells has been difficult in the 4NQO model relying on histopathology to detect neoplastic cells. Neither visible tumors nor histologic invasive SCC lesions emerge until 20 weeks from the start of 4NQO treatment (FIG. 13) (Natsuizaka et al. 2017; Tang et al. 2004). Both dysplasia and ESCC form multifocal lesions. To characterize live neoplastic cells throughout the ESCC development and progression, the 3D organoid assays were explored, which appeared to be highly sensitive to detect premalignant and ESCC cells as a single cell-derived spherical structures. In 3D organoids from murine esophagi, we could detect neoplastic 3D structures (FIG. 17) as early as 8 weeks from the start of 4NQO treatment and that neoplastic 3D structures propagated rapidly when passaged (FIG. 17), indicating 4NQO-induced neoplastic cells show enhanced self-renewal and proliferation compared to 4NQO-untreated normal esophageal cells. Histology of 3D organoids revealed predominantly low-grade dysplasia at 8 week, which progressed to high-grade dysplasia by 16 week from the start of 4NQO. 3D organoid assays were further performed to detect metastatic ESCC cells (FIGS. 18A and 18B). In cell lineage-traceable mice treated with 4NQO, para-esophageal lymph nodes lung and liver metastatic lesions developed along with primary ESCC tumors (FIG. 13 and FIG. 14) where YFP-labeling confirmed esophageal epithelial origin of the resulting 3D organoids (FIG. 15, panel d). Given co-existing non-esophageal cells, the organoid formation rate (OFR) was lower for primary organoids from metastatic lesions (e.g. 0.01% from the lung vs. 5-10% from the esophagus); however, non-esophageal cells did not grow in our culture conditions and that metastatic 3D organoids propagated rapidly when passaged. Interestingly, lung metastatic ESCC 3D organoids displayed highly irregular-shaped structures and increased proliferation compared to those from the esophagus (primary tumors) and lymph nodes (FIGS. 18A and 18B). 3D organoids from mice with esophagitis-related reactive epithelial changes (basal cell hyperplasia) displayed a normal proliferation-differentiation gradient in the absence of an inflammatory milieu ex vivo (Whelan et al. 2017; Kasagi et al. 2018). Thus, the neoplastic 3D structures from 4NQO-treated mice reflect uniquely carcinogen-induced intrinsic changes of cells. In aggregate, the 3D organoid assays will offer an unprecedented robust analyses of live cells from premalignant to metastatic lesions, thus promising a substantial expansion of applications in mouse models of ESCC.

### Patient-Derived 3D Organoids (PDOs) Recapitulate the Original Tissues

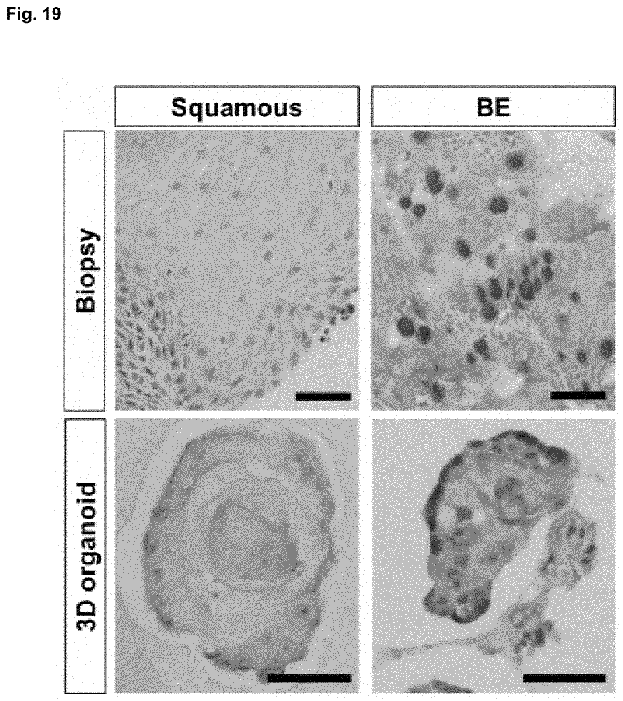

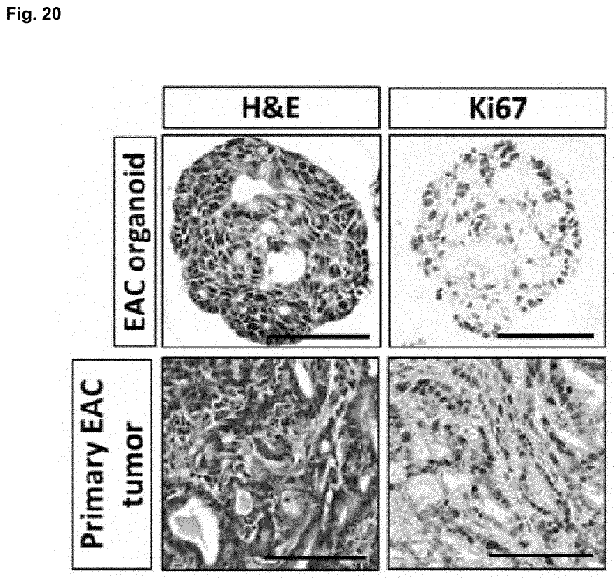

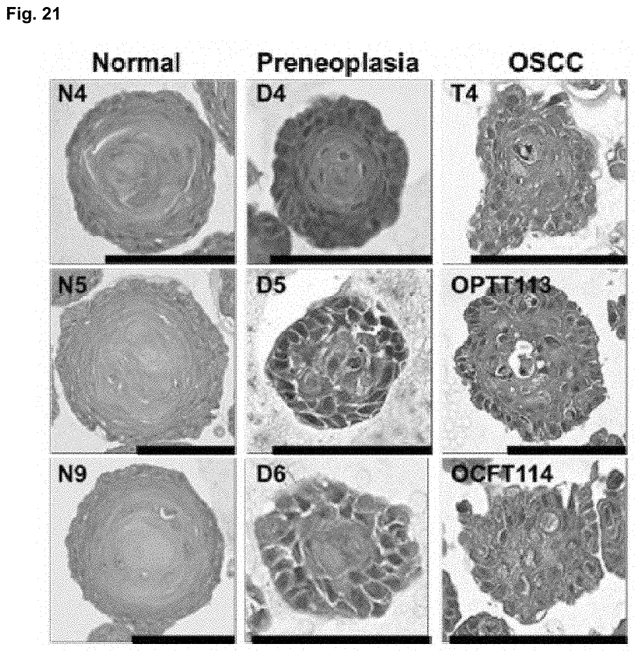

Application of the PDO system for human esophageal epithelial cells have met technical difficulties. Although Sato et al. were the first to report the generation of human BE tissue-derived organoids, they did not provide a success rate of organoid formation (Sato et al. 2011). Moreover, their culture conditions did not permit growth of normal human esophageal 3D organoids with squamous-cell differentiation, preventing studies on regenerative neo-squamous islands (NSI) induced after radiofrequency ablation of BE containing neoplastic lesions (Pouw et al. 2009). Culture conditions were optimized to be permissive for generation of PDOs from both normal and neoplastic esophageal lesions in patients' biopsies or surgically resected tumor tissues (Nakagawa et al. 2020; Karakasheva et al. 2020; Kasagi et al. 2018; Kijima et al. 2019). The improved culture conditions allow now generation of PDOs from both BE and adjacent normal mucosal biopsies (FIG. 19), recapitulating distinct cellular characteristics (i.e. squamous-cell differentiation vs. intestinal metaplasia) in parental tissues. A library of PDOs was built from >30 IRB-approved subjects with normal esophageal mucosa, GERD and eosinophilic esophagitis. PDOs were established from patients with BE, EAC, ESCC and oral SCC (FIG. 20 and FIG. 21).

### Detection of Super-Early Neoplastic Changes and Cancer Cell Development/Progression

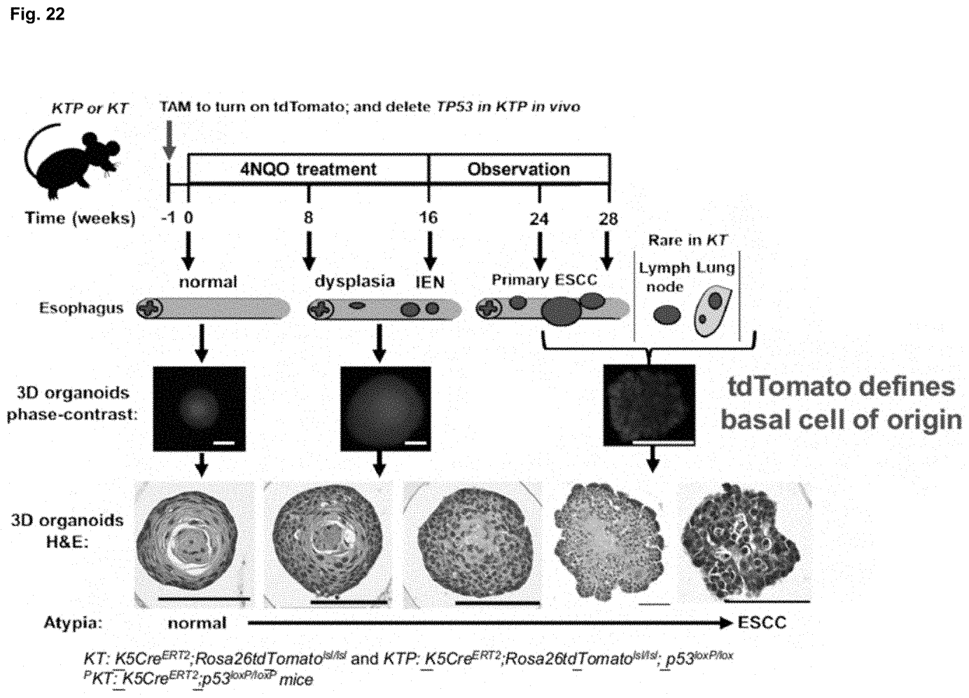

As shown in FIG. 22, we have evaluated the impact of loss of tumor suppressor gene TP53 in the esophageal epithelium in mice treated with the oral-esophageal carcinogen 4NQO. We have utilized two genetically engineered mouse strains KTP and KT. KTP (experimental) mice carry a conditional TP53 alleles p(53^{loxP/loxP}) in homozygosity, along with Rosa26 homozygous loci knocked-in with a gene encoding tdTomato (tdT) fluorescent protein. KT (control) mice have tdT only. Both KTP and KT mice carry a tamoxifen (TAM)-inducible transgenic Cre^{ERT2} (Cre recombinase) targeted to the the esophageal epithelial cells via the cytokeratin KRT5 promoter. Mice received TAM a week before the start of 4NQO treatment. TAM activates Cre^{ERT2}, resulting in induction of tdTomato protein in the oral-esophageal epithelia of both KTP and KT mice along with concurrent deletion of TP53 in KTP, but not KT, mice. Following the 4NQO-treatment period, mice were subjected to observation for tumor development. As shown in FIG. 13, mice did not have invasive squamous cell carcinoma (SCC) until the 24-week time points. Metastasis of SCC was detected in KTP, but rarely in KT, mice at the end of the observation period. We have generated 3D organoids from esophagi at variable time points to compare morphological differences. tdTomato expression confirms that the resulting organoids were originated from the epithelia with tdTomato expression. 3D organoids generated from normal (4NQO-untreated) mice at time 0 shows a concentric structure with an apparent differentiation gradient as documented by H&E staining. 3D organoids displayed increasingly more cellular atypism as a function of time. At the stage of SCC (ESCC, esophageal squamous cell carcinoma as an example), tdTomato-positive organoids appeared to be highly lobulated with a high degree of cell atypism as well as a lack of differentiation and an increase in proliferation.

Esophageal 3D organoids were generated from TAM-treated KTP and KT mice (FIG. 22) at 4 weeks after 4NQO treatment, a time point when conventional histologic evaluation (FIG. 13) may not detect neoplastic epithelial changes in the oral and esophageal mucosa. Esophageal cells isolated from KTP mice (with loss of TP53) displayed lower organoid formation rate compared to control KT mice. Once grown, however, KTP-derived organoids grew larger compared to KT-derived organoids. Moreover, KTP-derived, but not KT-derived, 3D organoids displayed basal cell atypia despite a differentiation gradient, suggesting that loss of the tumor suppressor TP53 may have accelerated 4NQO-induced neoplastic changes in KTP mice. Additionally, this experiment suggests that 3D organoids may be more sensitive to detect neoplastic changes than the conventional histopathology method.

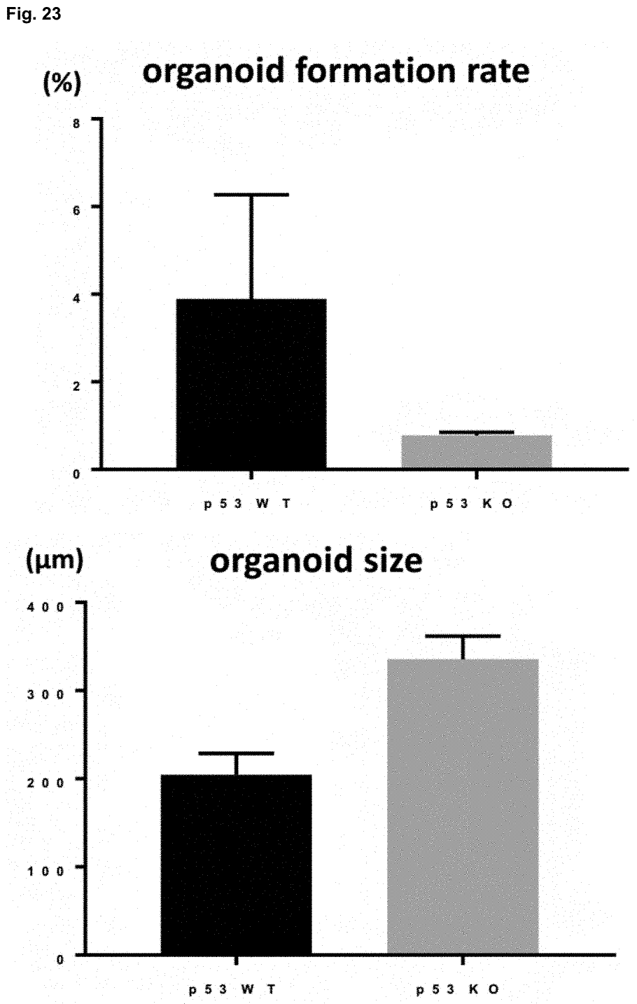

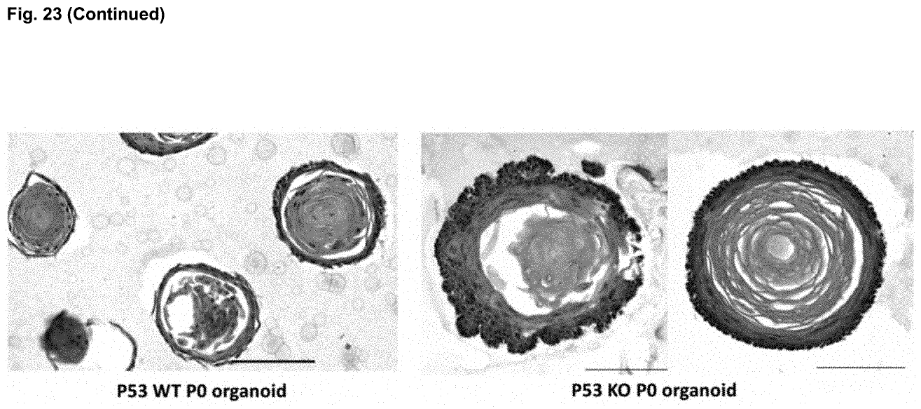

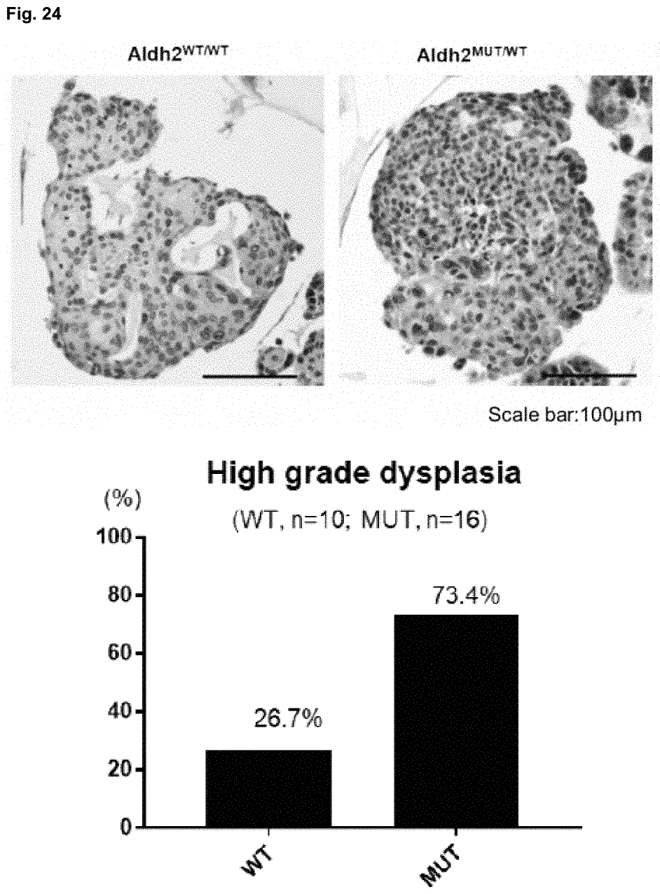

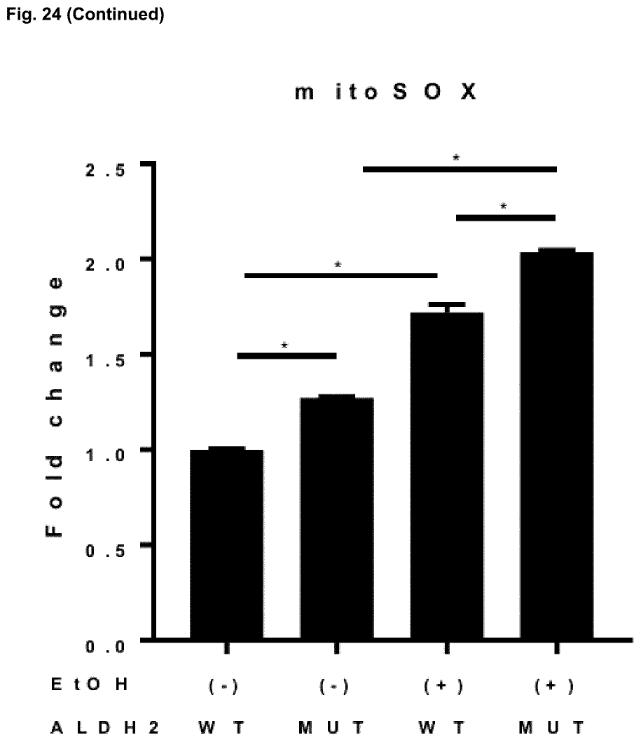

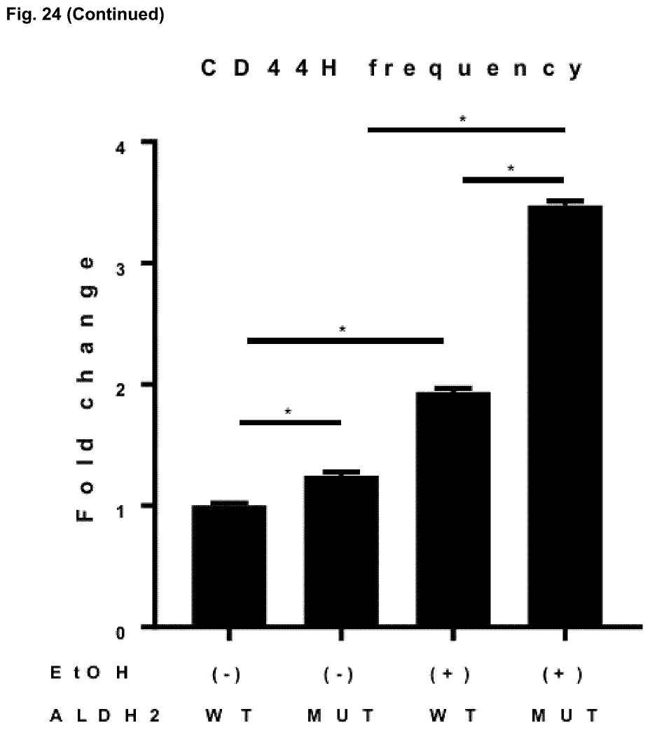

FIG. 23 shows a comparison of 3D organoids generated from 4NQO-treated mice carrying Aldh2 mutation (Aldh2^{Mut/WT}) or wild-type Aldh2 (Aldh2^{WT/WT}) alleles, encoding a mitochondrial enzyme Aldh2 essential in alcohol metabolism. In Aldh2^{Mut/WT} mice, the mutant Aldh2 diminishes the capability to clear toxic alcohol metabolites including acetaldehyde, a genotoxic chemical compound and a human carcinogen. Acetaldehyde is a key alcohol metabolite as well as a constituent of tobacco smoking, the latter mimicked by 4NQO treatment. Aldh2 mutations increased the cellular atypism (dysplasia) in 3D organoids generated from 4NQO-treated Aldh2^{Mut/WT} mice compared to Aldh2^{WT/WT} control mice. Additionally, the dysplastic 3D organoids from Aldh2^{Mut/WT} mice displayed a stronger response to alcohol (ethanol, EtOH) exposure in culture, producing the higher level of mitochondrial superoxide (MitoSOX), resulting in enrichment of a unique subset of cells expressing a high level of CD44 (CD44H cells), a marker of oral-esophageal tumor initiating cells. Thus, the experimental results in FIG. 24 show that organoids are useful to test how life-style related risk factor may influence neoplastic changes from individuals with differential genetic factors.

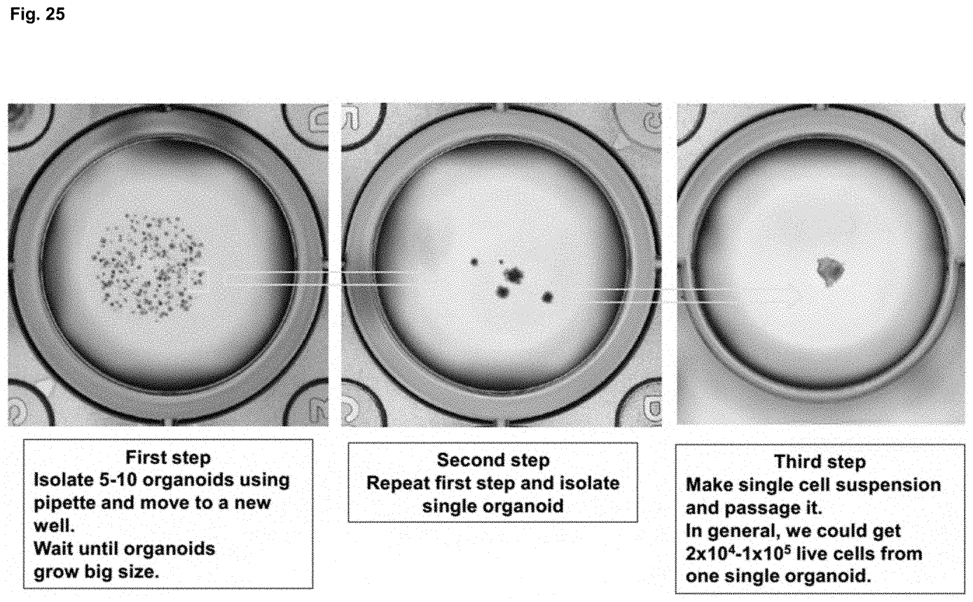

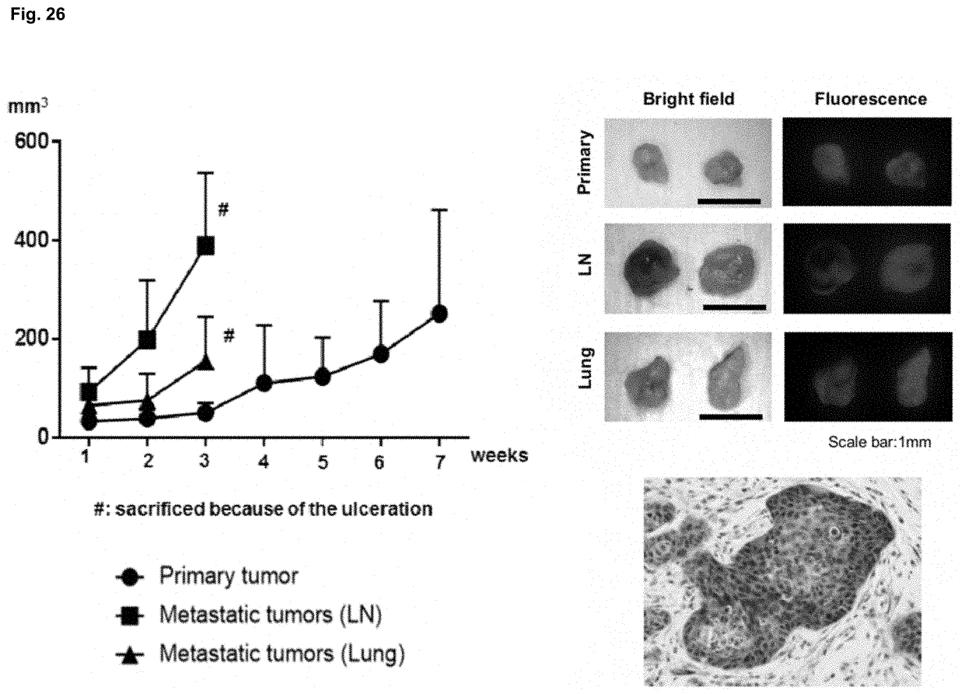

As shown in FIG. 26, we have utilized 4NQO-treated KTP mice with TAM-induced tdTomato expression and concurrent loss of TP53 (FIG. 22). Mice bore primary esophageal tumors (ESCC) and metastatic tumors in the lymph node (LN) or the lung. We generated tdTomato-positive 3D organoids from primary tumors (primary) and metastatic lesions LN or lung. Individual organoid clones were isolated by the technique illustrated in FIG. 25. We validated morphologically that each organoid clone displays ESCC-compatible high grade atypism (FIG. 22 and FIG. 17). Then, each organoid clone was expanded in culture and subjected to tumor formation assays using immunodeficient mice. We monitored tumor growth and plotted in the graphs shown. Organoid clones from metastatic lesions appeared to be more tumorigenic than those from primary tumors. The upper right panel shows representative tumors isolated from the mice at the end when mice were sacrificed. tdTomato expression confirms that the origin of tumors. Histology (lower right) documents poorly-differentiated SCC from ESCC organoid-derived tumors.

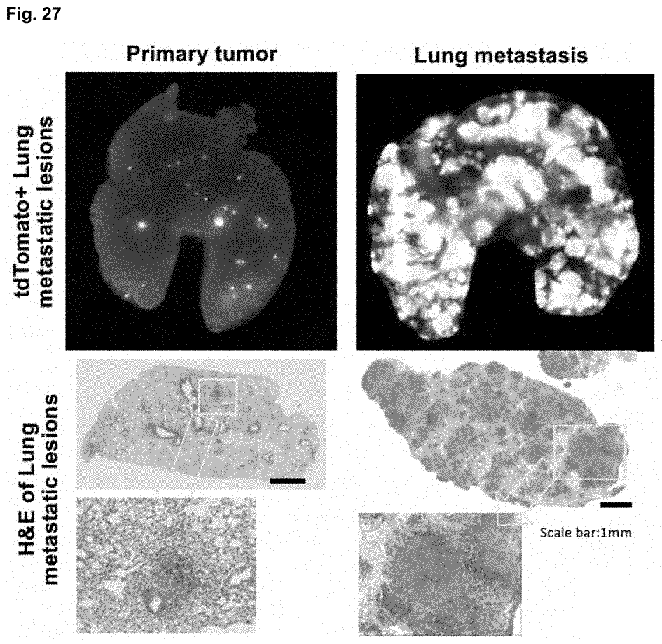

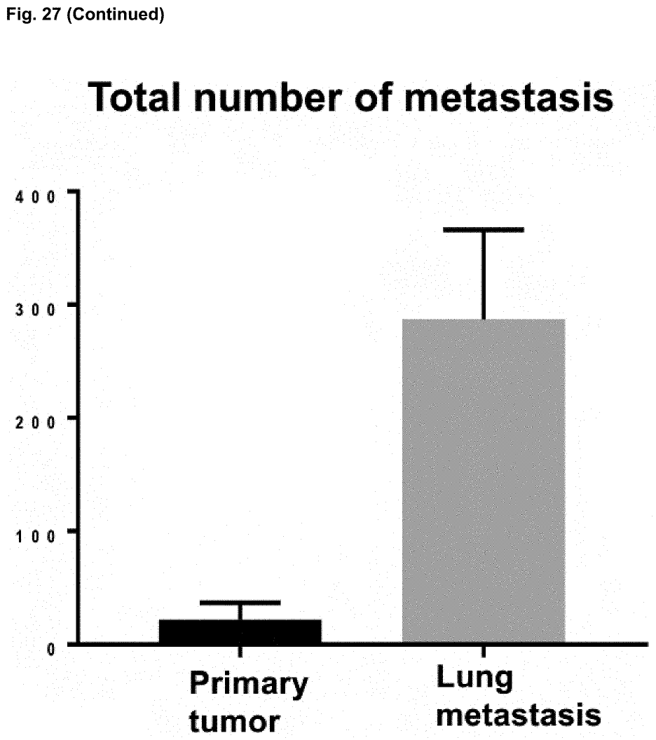

As shown in FIG. 27, the organoid clones isolated from a primary ESCC tumor or a lung metastatic lesion (FIG. 26) were used to evaluate their metastatic potential in immunodeficient mice via the tail-vein injection assays. When mice were sacrificed, lung metastatic lesions were evaluated by fluorescence dissection microscopy that detected tdTomato fluorescent-positive metastatic lesions in the lung and quantitated as shown in the bar graph to the right. The organoid clone isolated from lung metastatic lesions appeared to be far more capable of reestablishing the metastatic lesions in the lung compared to that from a primary tumor. tdTomato-positive metastatic organoid-tumor lesions were validated by H&E staining.

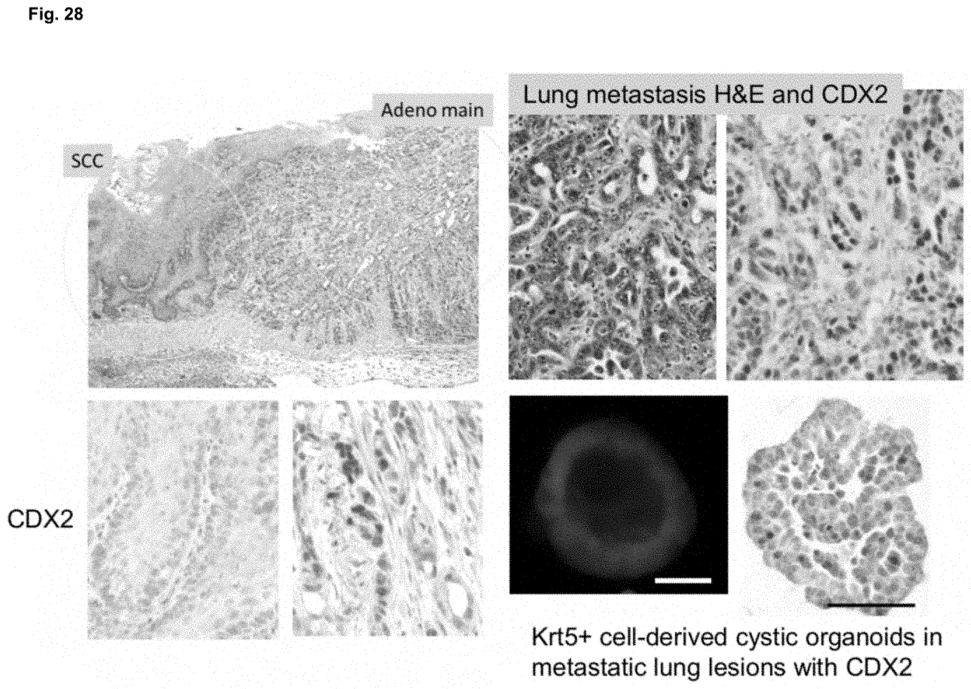

We have also analyzed adenosquamous cell carcinoma (ASC), a rare form of esophageal cancer comprising squamous cell carcinoma and adenocarcinoma (adeno), two distinct histopathologic tumor cell types (FIG. 28). In this experiment, mice had both a primary esophageal tumor and lung metastatic lesions. Adenocarcinoma was documented by immunohistochemistry for CDX2, a marker of intestinal cell type. Organoids (FIG. 28, right lower panels) showed tdTomato expression and CDX2 expression. In cell lineage traceable mice, tdTomato expression indicates cytokeratin K5 (Krt5)-positive squamous epithelial cells as origin of tumor cells. CDX2 expression indicates the cystic organoid structure consists of adenocarcinoma-compatible cancer cells.

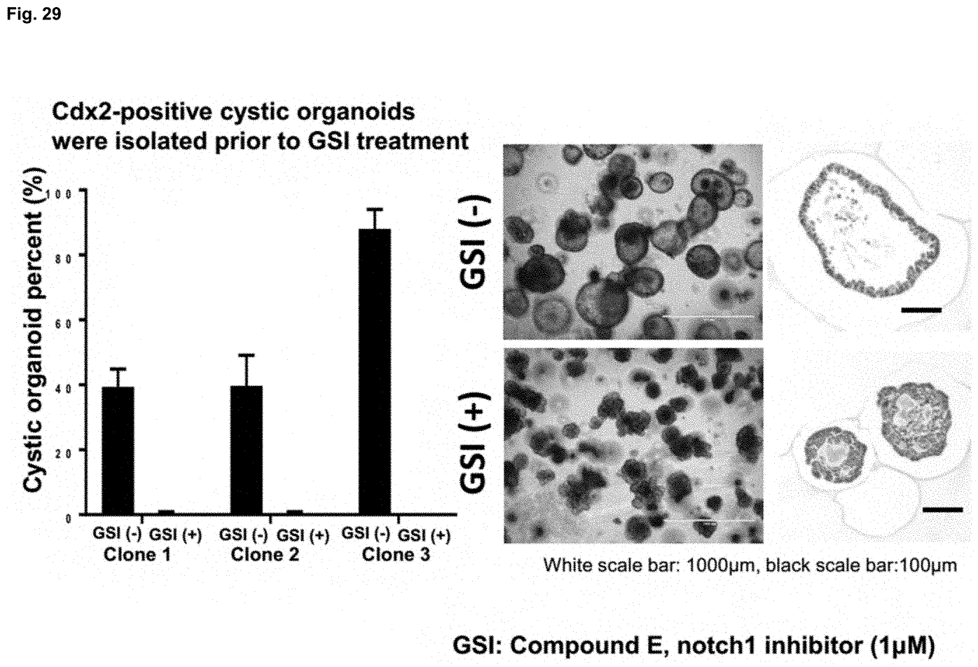

As shown in FIG. 29, the adenocarcinoma-like organoids (FIG. 28) were also treated with gamma-secretase inhibitor (GSI), a pharmacological inhibitor of Notch signaling that regulates cell fate. GSI treatment converted cystic organoids to non-cystic keratinized structures that are compatible with squamous cell carcinoma. Therefore, Notch signaling may regulate cell plasticity of adenocarcinoma cells to be converted to squamous carcinoma cells in ASC.

### Patient-Derived 3D Organoids (PDOs) Serve as Modeling Tools for Oral and Esophageal Preneoplastic and Cancer Lesions and Personalized Medicine

The esophageal patient-derived 3D organoid (PDO) system was employed as near-physiological experimental platforms to study esophageal biology under homeostatic and pathologic conditions (Whelan et al. 2018). PDOs are initiated directly following dissociation of live tissues containing stem/progenitor cells or tumor-initiating cells. PDOs grow in basement membrane (i.e. Matrigel™) under submerged conditions to recapitulate the original tissues. By passaging dissociated primary structures to generate single cell-derived secondary 3D organoids, this system can be utilized to validate the self-renewal activities of putative stem cells. Using patients' biopsies, esophageal PDO has been transformative with great potential to advance personalized medicine, for example, by testing chemotherapeutic sensitivity of EAC PDOs from individual patients. PDOs will be used as a common platform to compare differences between disease stages during the development and progression of BE and EAC. If successful, this will represent a significant advance in the field of esophageal cancer biology, providing a tool to not only evaluate human relevance of the BE and EAC stem/progenitor cells identified in mice, but predict therapeutic response and optimize treatment strategies in a personalized manner.

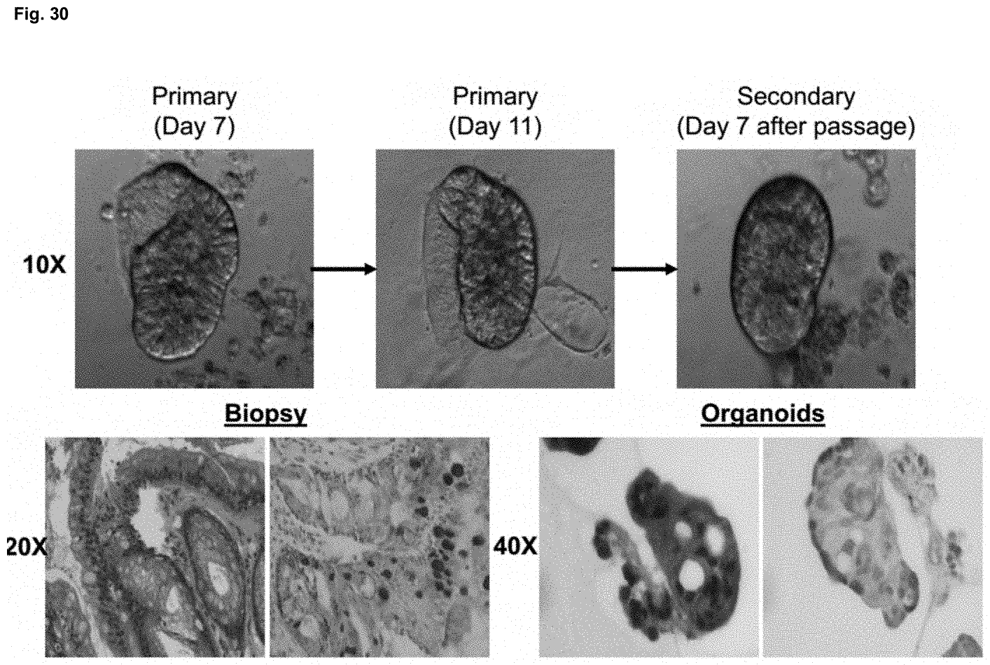

As shown in FIG. 30, we have generated and passaged patient-derived 3D organoids from IRB-approved endoscopic biopsies from patients with intestinal metaplasia or Barrett's esophagus (BE), a precursor lesion for esophageal adenocarcinoma. Histology images below show that organoids (right) recapitulate morphology of the original biopsy sample (left). Alcian blue staining detects cells containing mucin, a sign of goblet cell metaplasia, a feature of intestinal metaplasia/BE.

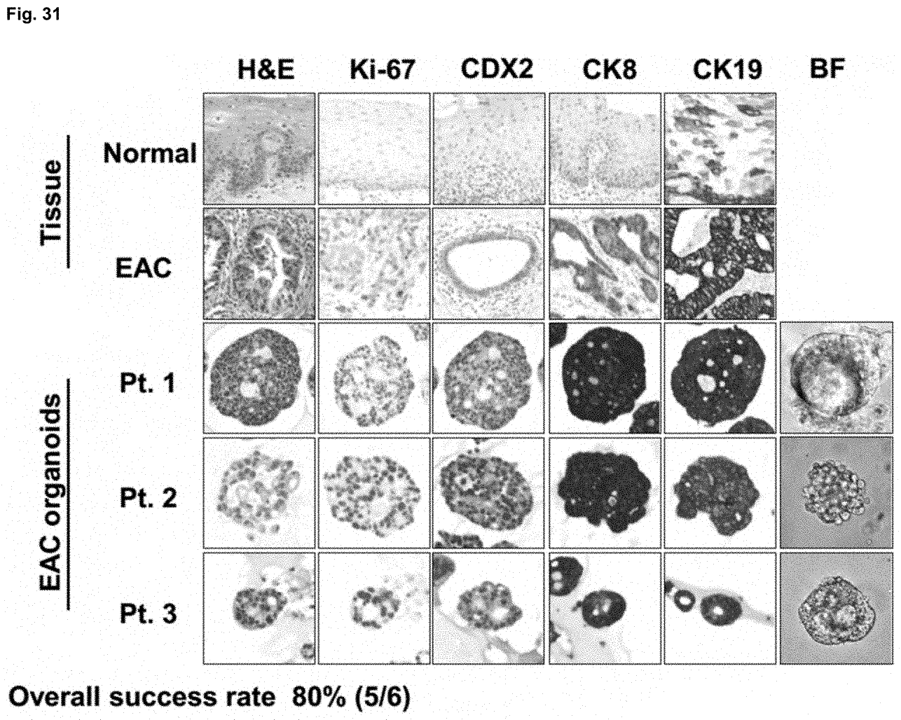

We have also generated and characterized 3D organoids representing esophageal adenocarcinoma (EAC) from three patients (FIG. 31). Positive expression for CDX2, CK8 and CK19, markers of EAC cells, as confirmed in the original tissues, validate the EAC organoids. (BF, bright field images of the organoid structures)

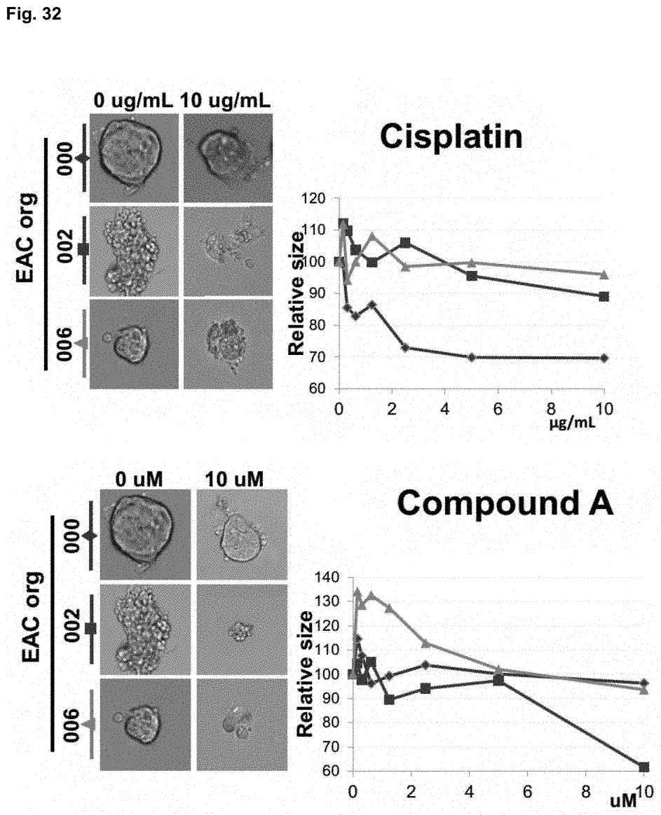

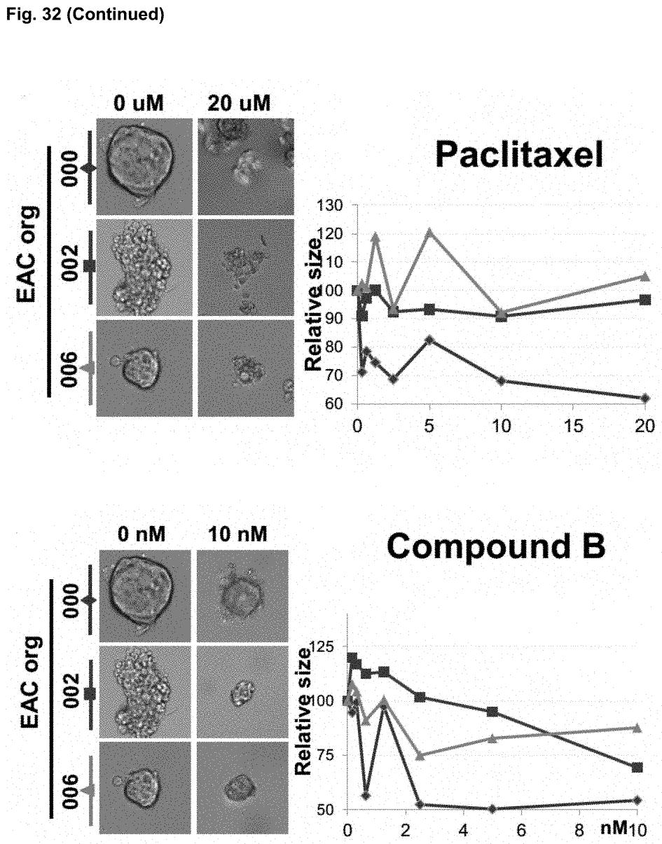

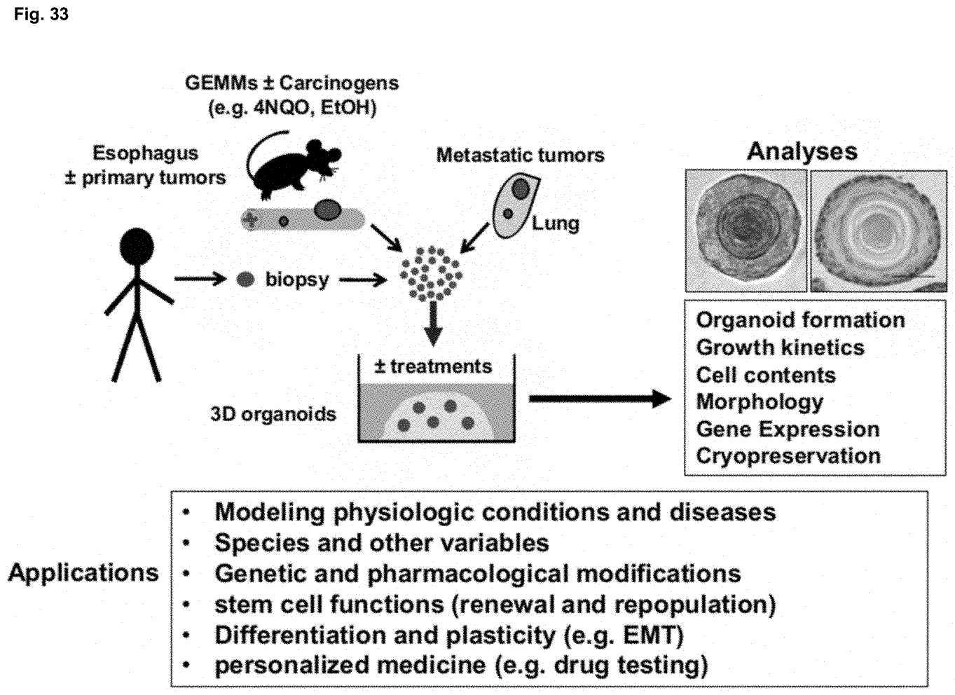

Moreover, as shown in FIG. 32, patient-derived EAC organoids (3 patients: red, blue and grey) were treated with indicated anti-cancer drugs, showing individually-differential drug response.

### Example 3

### Modeling Epithelial Homeostasis and Reactive Epithelial Changes in Human and Murine Three-Dimensional Esophageal Organoids

The esophagus connects the oral cavity and the stomach. The surface is lined by layers of stratified squamous epithelium, and renews through continuous cell division (proliferation) and differentiation followed by loss (desquamation) from the outmost layer into the lumen. This provides barrier against the luminal contents including acid, microorganisms, and food allergens. Under disease conditions, a variety of leukocytes are recruited to the esophagus to cause inflammation and produce cytokines. In response to cytokines, epithelial cells show reactive changes, resulting in impaired barrier function and aggravated inflammation. This Example describes protocols to reconstitute human and mouse esophageal epithelial structures in a highly efficient novel 3D cell culture to model and analyze epithelial homeostasis and perturbation under disease conditions such as esophagitis.

The homeostatic proliferation-differentiation gradient in the esophageal epithelium is perturbed under inflammatory disease conditions such as gastroesophageal reflux disease and eosinophilic esophagitis. Herein we describe the protocols for rapid generation (<14 days) and characterization of single cell-derived three-dimensional (3D) esophageal organoids from human subjects and mice with normal esophageal mucosa or inflammatory disease conditions. While 3D organoids recapitulate normal epithelial renewal, proliferation and differentiation, non-cell autonomous reactive epithelial changes under inflammatory conditions are evaluated in the absence of the inflammatory milieu. Reactive epithelial changes are reconstituted upon exposure to exogenous recombinant cytokines. These changes are modulated pharmacologically or genetically ex vivo. Molecular, structural and functional changes are characterized by morphology, flow cytometry, biochemistry and gene expression analyses. Esophageal 3D organoids can be translated for the development of personalized medicine in assessment of individual cytokine sensitivity and molecularly-targeted therapeutics in esophagitis patients. The following protocols are provided in this Example:
Basic Protocol 1: Generation of esophageal organoids from biopsy or murine esophageal epithelial sheets
Basic Protocol 2: Propagation and cryopreservation of esophageal organoids
Basic Protocol 3: Harvesting of esophageal organoids for RNA isolation, immunohistochemistry and evaluation of 3D architecture
Basic Protocol 4: Modeling of reactive epithelium in esophageal organoids Support Protocol: Procurement of murine esophageal epithelial sheets

### Introduction

Epithelium is the barrier between the body and the outside world. A stratified squamous epithelium lines the esophageal mucosa to provide protection against mechanical trauma from food, chemical injury from acids in the luminal content, immunogenic food allergens, as well as invading pathogens (Rosekrans et al., 2015). The stratified squamous epithelium of the esophagus contains a basal layer of proliferative and undifferentiated epithelial cells (basal keratinocytes). These cells undergo post-mitotic terminal differentiation (cornification) within the overlying suprabasal cell layers to form stratified squamous epithelium (Rosekrans et al., 2015). The esophageal epithelium maintains an exquisite balance of proliferation and differentiation through continuous proliferation of basal keratinocytes, migration of differentiated keratinocytes toward the luminal surface, and finally desquamation of cornified and flat keratinocytes, allowing epithelial renewal that occurs within a few weeks (Whelan et al., 2018).

Inflammation, as seen under disease conditions such as gastro-esophageal reflux disease (GERD) and eosinophilic esophagitis (EoE), disrupt the afore-mentioned homeostatic balance of proliferation and differentiation causing subsequent barrier disruption and prolonged mucosal injury. It is therefore imperative to develop a reliable ex vivo model to recapitulate the epithelial differentiation gradient of the esophageal epithelium in humans and mice in order to study these diseases in their physiologically-relevant three-dimensional (3D) context (Whelan et al., 2018).

The single cell-derived esophageal 3D organoid culture system was established to mimic in vivo tissue architecture to model epithelial homeostasis and reactive changes associated with esophageal disease conditions (Kasagi et al., 2018). Protocols were provided to (1) prepare single cell suspensions from (a) endoscopic biopsies from human subjects or (b) epithelial sheets isolated from murine esophagi as starting materials; (2) grow, passage and cryopreserve 3D organoids; (3) determine growth kinetic of 3D organoids; and (4) perform morphological and functional evaluation of 3D organoids coupled with or without pharmacologic or genetic manipulations.

### Strategic Planning

### Human Subjects

For human esophageal 3D organoid studies, it is imperative to have (1) the Health Insurance Portability and Accountability Act (HIPAA) authorization, as well as an Institutional Review Board (IRB)-approved protocol; (2) an appropriate clinical care facility (e.g. endoscopy unit); (3) a clinical care team comprising physicians/gastroenterologists, nurses, support staff, research nurses/clinical coordinators who recruit consented human subjects and procure esophageal tissue samples; (4) coordination with laboratory personnel who should be notified in advance to schedule time to initiate 3D organoid culture; and (5) proper safety training of laboratory personnel. IRB protocol should be carefully planned to permit multiple research biopsies for concurrent histopathological analyses and an access for the clinical information (e.g. age, gender, endoscopic findings, pathology reports, therapy outcome).

During routine upper endoscopy (esophagogastroduodenoscopy), an expert gastroenterologist should perform a routine esophageal biopsy with forceps and concurrent photo-documentation of the esophageal mucosa. Biopsy/surgical specimens (submerged in sterile PBS) need to be transported as soon as possible (<2 hours) to a research lab for tissue processing and the initiation of 3D organoids culture. Laboratory personnel should have proper knowledge and laboratory safety training about infectious agents including hepatitis and human immunodeficiency viruses that may be potentially present in the starting materials.

To compare esophageal 3D organoids grown from different individuals for parameters such as organoid formation rate and growth rate, it is important to include a quality control cell line (e.g. immortalized normal human esophageal cell line such as EPC2-hTert) (Harada et al., 2003; Muir et al., 2013) to minimize the influence of variables such as cell culture medium components, cell culture incubator conditions, and experience levels of experimenters.

### Mice

For murine esophageal 3D organoid studies, experiments must be planned and performed in accordance with regulations and an approved protocol under a local regulatory body (e.g. Institutional Animal Care and Use Committee and Animal Ethics Committee). Mice should be housed at a proper animal care facility that ensures humane treatment of mice and provides appropriate veterinary care of mice and laboratory safety training of laboratory personnel.

Biological replicates include sets of 3D organoids generated from independent mice. As organoids can be manipulated ex vivo pharmacologically or genetically for evaluation, a minimal number of mice (n=2) is generally sufficient per experiment to ensure multiple technical replicates (n=3-6). If necessary, pilot studies should be performed to estimate appropriate sample size of mice permitting detection of medium to large effects with 80% power in experiments planned.

### Basic Protocol 1

Generation of Esophageal Organoids from Biopsy or Murine Esophageal Epithelial Sheets

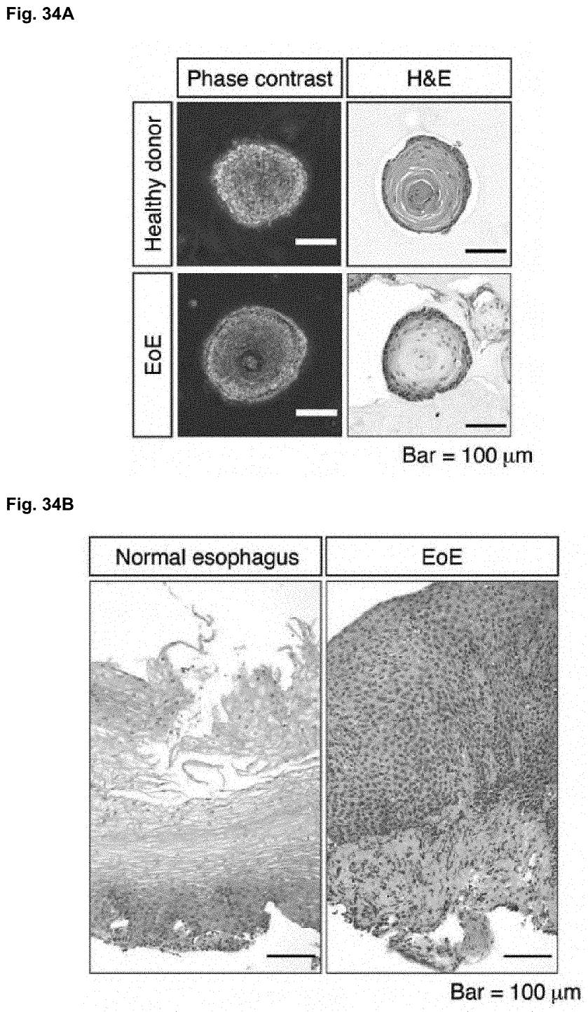

Tissue specimens were obtained either via biopsy (for human patients) or necropsy (for murine esophagi) and dissociated enzymatically and mechanically, followed by embedding of single cells in Matrigel. The organoids are cultured for 11 days, resulting in formation of 3D spherical structures recapitulating the original tissue. Representative images of esophageal organoids generated from EoE patient and a healthy control donor can be found in FIGS. 34A-34B. Murine organoids can be cultured in the same medium as human patient-derived organoids.

All tools should be properly cleansed and sterilized prior to use. All plasticware and glassware should be cell culture grade and disposable. Water and DMSO used to dissolve or dilute reagents should be ultrapure and sterile. Standard equipment and tools for cell culture (CO₂ incubator, tissue culture hoods, liquid nitrogen cell storage tank, vacuum aspirator/collection system, centrifuges, pipettors, etc.) are needed. While alternatives are available, key items used routinely in the laboratories were listed.

### Materials

Human tissue fragment (single human patient biopsy punch; human tissue fragments obtained through endoscopy at Children's Hospital of Philadelphia; see Strategic Planning, Human Subjects section)
Penicillin-streptomycin (Thermo Fisher Scientific, cat. no. 15140122)
Keratinocyte serum-free medium (KSFM; Thermo Fisher Scientific, cat. no. 10724-011) supplemented with 1 ng/ml epidermal growth factor (EGF), 5 μg/ml bovine pituitary extract (BPE), and penicillin/streptomycin
Dispase (50 U/ml; Corning, cat. no. 354235), stored undiluted at −2° C. in 1 ml aliquots
HBSS (Thermo Fisher Scientific, cat. no. 14175079)
Dulbecco's phosphate-buffered saline (1×PBS; Thermo Fisher Scientific, cat. no. 14190250)
0.05% trypsin-EDTA (Thermo Fisher Scientific, cat. no. 25-300-054)
250 μg/ml soybean trypsin inhibitor (MilliporeSigma, cat. no. T9128)
Trypan Blue Stain (Thermo Fisher Scientific, cat. no. T10282)
Matrigel basement membrane matrix (Corning, cat. no. 354234)
10 μM Y27632 (Selleck Chemicals, cat. no. S1049)
Gibco amphotericin B, 0.5 μg/ml (Thermo Fisher Scientific, cat. no. 15290018)
4% paraformaldehyde (MilliporeSigma, cat. no. 158127-500G)
Calcium chloride, anhydrous (Thermo Fisher Scientific, cat. no. AC349615000)
300 mM calcium chloride (CaCl₂)) solution (dissolve 33.3 g calcium chloride in 1 L water, filter-sterilize, and store at 4° C.)
KSFM with calcium (KSFMC; add 1 ml 300 mM CaCl₂ solution to 500 ml KSFM supplemented with BPE, EGF, and penicillin/streptomycin)
1.5-ml microcentrifuge tube
ThermoMixer C (Thermo Fisher Scientific, cat. no. 14-285-562 PM)
Centrifuge Sorval ST 16R (Thermo Fisher Scientific, cat. no. 75004380)
Microcentrifuge Minispin (Eppendorf, cat. no. 022620100)
Countess II FL Automated Cell Counter (Thermo Fisher Scientific, cat. no. AMQAX1000)
Forceps (VWR, cat. no. 82027-386)
1-ml tuberculin syringe (BD, cat. no. 309659)
70-μm cell strainer (Thermo Fisher Scientific, cat. no. 22363548)
40-μm cell strainer (Thermo Fisher Scientific, cat. no. 22363547)
50-ml conical tube (Thermo Fisher Scientific, cat. no. 12-565-270)
5-ml round bottom polystyrene tube with cell strainer snap cap (BD, cat. no. 352235)
24-well plate (Thermo Fisher Scientific, cat. no. 12-556-006)
60-mm cell culture dish (Thermo Fisher Scientific, cat. no. 12-556-001)
   Procure Tissue and Dissociate into a Single-Cell Suspension
1. Place tissue fragment (a single human patient biopsy punch) in a 1.5-ml microcentrifuge tube containing 750 μl cold KSFM and transfer to lab on ice.
2. Remove KSFM, incubate biopsy in 1 ml dispase (10 U/ml) 10 min at 37° C. in thermomixer (700-800 rpm). To prepare working solution of dispase, dilute the 50 U/ml stock in HBSS at a 1:5 ratio.
3. Remove dispase and wash tissue three times with 1 ml 1×PBS.
4. Incubate biopsy with 500 μl 0.05% trypsin (heated to 37° C. prior to addition) at 37° C. for 10 min in thermomixer (700-800 rpm). When generating murine esophageal organoids, begin at this step (see Support Protocol for preparation of murine esophageal epithelial sheets).
5. Place 70-μm cell strainer on top of a 50-ml conical tube.
6. Pre-load cell strainer with 2 ml 250 μg/ml soybean trypsin inhibitor.
7. Use a pipet to transfer isolated cells and add dissociated tissue to strainer.
8. Use plunger head of 1-ml tuberculin syringe to force remaining cells and tissue fragments through the strainer. Place the strainer in a 60-mm cell culture dish to prevent membrane damage.
9. Rinse strainer with 1 ml 250 μg/ml soybean trypsin inhibitor.
10. Filter cell suspension into a 5-ml round bottom polystyrene tube with a cell strainer cap.
11. Pellet cell suspension at 500×g for 5 min (using Sorval ST centrifuge).
12. Aspirate 2.5 ml supernatant, re-suspend pellet in KSFM, and transfer to a 1.5-ml microcentrifuge tube.
13. Pellet in the mini centrifuge at 500×g for 3 min.
14. Re-suspend pellet in 50-100 μl KSFM, keep on ice, and count cells.

### Organoid Seeding and Culture in 24-Well Plates

15. Thaw and keep Matrigel on ice.
16. Pre-warm a 24-well plate at 37° C.
17. Plate 2,000 cells per well of a 24-well plate in 50 μl 1:1 Matrigel/KSFMC droplet.
18. Incubate droplets at 37° C. for 15 min.
19. Add 500 μl KSFMC to each well. Supplement KSFMC on day 0 with 0.5 μg/ml amphotericin B and 10 μM Y27632. (Addition of Y27632 improves viability of esophageal keratinocytes in single-cell suspension.)
20. Change medium on day 1, 4, 6, 8, and 10. The organoids are ready for harvest and/or passage on day 11.

### Basic Protocol 2

### Propagation and Cryopreservation of Esophageal Organoids

Once the organoids are established, esophageal keratinocytes can be isolated from the primary culture by enzymatic digestion and used for subsequent passaging and propagation. The isolated esophageal keratinocytes can be cryopreserved for long-term storage.

Additional Materials (see also Basic Protocol 1)
Growing culture of esophageal organoids (see Basic Protocol 1)
0.25% trypsin-EDTA (Thermo Fisher Scientific, cat. no. 25-200-056)
DNAse I (MilliporeSigma, cat. no. 10104159001)
FBS (e.g., HyClone, cat. no. SH30071.03)
Dimethyl sulfoxide (DMSO; MilliporeSigma, cat. no. D4540)
Nalgene general long-term storage cryogenic tubes (Thermo Fisher Scientific, cat. no. 03-337-7D)
CoolCell LX freezing container (Corning, cat. no. 432002)

### Organoid Disintegration and Preparation of Single-Cell Suspension

1. Digest Matrigel by adding 400 μl dispase per well and incubating 15 min at 37° C.
2. Transfer digested suspension to a 1.5-ml microcentrifuge tube.
3. Pellet suspension at 500×g for 5 min; remove supernatant.
4. Incubate with 0.25% trypsin supplemented with 10 μM Y27632 and 0.5 U/ml DNase I for 10 min at 37° C. in thermomixer. DNAse I prevents clumping of cells by DNA strands released from dying cells.
5. Place 40-μm cell strainer on top of a 50-ml conical tube.
6. Pre-load cell strainer with 1 ml 250 μg/ml soybean trypsin inhibitor.
7. Filter cell suspension through the strainer.
8. Rinse strainer with 1 ml 250 μg/ml soybean trypsin inhibitor.
9. Pellet cells at 500×g for 5 min.
10. Aspirate supernatant, resuspend in 1 ml KSFM, and transfer to a 1.5-ml microcentrifuge tube.
11. Pellet cells using mini centrifuge at 500×g for 3 min.
12. Resuspend pellet in 50-100 μl KSFMC, keep on ice, and count cells.

### Passage to 24-Well Plates

13. Thaw and keep Matrigel on ice.
14. Pre-warm a 24-well plate at 37° C.
15. Plate 2,000 cells per well of a 24-well plate in 50 μl 1:1 Matrigel/KSFMC droplet.
16. Incubate droplets at 37° C. for 15 min.
17. Add 500 μl KSFMC to each well. Supplement KSFMC on day 0 with 0.5 μg/ml amphotericin B and 10 μM Y27632.
18. Change medium on day 1, 4, 6, 8, and 10.

### Cryopreserve Esophageal Organoids

19. Prepare freezing medium by mixing 9 ml FBS with 1 ml DMSO; this medium can be stored at −20° C. Any brand of FBS can be used for this purpose.
20. Resuspend cell pellet from step 11 in freezing medium to the final concentration of 10⁵ cells/ml.
21. Dispense 1 ml suspension from step 20 per cryovial.
22. Place cryovials in a freezing container and keep at −80° C. overnight.
23. Transfer frozen vials into a liquid nitrogen storage tank.
   Recovery after Cryopreservation
24. Thaw and keep Matrigel on ice.
25. Pre-warm a 24-well plate at 37° C.
26. Thaw cryovial in a 37° C. water bath ˜30 s (until the contents are liquid).
27. Transfer cell suspension to a microcentrifuge tube.
28. Pellet cells in the mini centrifuge at 500×g for 3 min; remove supernatant.
29. Resuspend cell pellet in 1 ml 1×PBS.
30. Pellet cells in the mini centrifuge at 500×g for 3 min; remove supernatant.
31. Resuspend pellet in 1 ml KSFM; assess cell density and viability.
32. Pellet cells in the mini centrifuge at 500×g for 3 min; remove supernatant.
33. Resuspend cell pellet in 1:1 Matrigel/KSFMC to final concentration of 4×10⁵ cells/ml and plate droplets of 50 μl/well from the 24-well plate.

### Basic Protocol 3

### Harvesting of Esophageal Organoids for RNA Isolation, Immunohistochemistry, and Evaluation of 3D Architecture

Histological evaluation and gene expression profiling are essential tools in research. This Example provided a protocol for fixation of esophageal organoids for embedding into paraffin blocks, as well as for RNA isolation from esophageal organoids.

Additional Materials (see also Basic Protocol 1)
Growing culture of esophageal organoids (see Basic Protocol 1)
Bacto agar (BD, cat. no. 214010)
Gelatin (Thermo Fisher Scientific, cat. no. G7-500)
Embedding gel (2% Bacto agar; 2.5% gelatin: Resuspend 1 g Bacto agar and 1.25 g gelatin in 50 ml water, swirl suspension, and let sit 30-60 min at room temperature; autoclave 20 min and aseptically dispense 5 ml per 15-ml conical tube, then store aliquots at room temperature)
Ethanol, 200 Proof (Thermo Fisher Scientific, cat. no. 22-032-601)
15-ml conical tubes (Thermo Fisher Scientific, cat. no. 14-959-53A)
Embedding pipet tips (cut end of a 200-μl pipet tip to make a bevel)
Parafilm M wrap (Thermo Fisher Scientific, cat. no. S37440)
Microcentrifuge tube rack (Southern Labware, cat. no. 0061)
Embedding mold surface (wrap a microcentrifuge rack in Parafilm to make a hydrophobic surface)
Tissue cassette (Thermo Fisher Scientific, cat. no. 22-272416)

### Harvest Organoids

1. Aspirate medium from wells.
2. Use 1,250 μl pipet tip to loosen Matrigel droplet attachment to well.
3. Scrape loose Matrigel droplet to bottom of well.
4. Transport Matrigel droplet to microcentrifuge tube.
5. Combine 3 droplets into one microcentrifuge tube.
6. Add 1×PBS and dispase (250 μl PBS mixed with 100 μl dispase).
7. Pipet vigorously 50 times to break up Matrigel droplets.
8. Vortex for 15 s.
9. Centrifuge 3 min at 500×g in microcentrifuge and rinse pellet with 1 ml 1×PBS
10. Resuspend by vortexing briefly and pellet again.

### Isolate RNA/Protein

11. For isolation of RNA or protein from esophageal organoids, add appropriate lysis buffer directly to pellet from step 10.

### Fix for Immunohistochemistry and Evaluate 3D Architecture

12. Fix esophageal organoid pellet from step 10 by resuspending in 500 μl 4% paraformaldehyde (PFA) and incubating at 4° C. overnight.
13. Discard 4% PFA, pellet organoids 3 min at 650×g in microcentrifuge.
14. Wash pellet with 1 ml 1×PBS, pellet organoids 3 min at 650×g in microcentrifuge, and aspirate as much liquid as possible with a pipet without disturbing pellet. Small amounts of residual liquid (e.g., 10 μl) are acceptable; however, larger volumes will dilute the embedding gel and can complicate sample processing.
15. Liquify embedding gel: Place the 15-ml conical tube with solid gel into a 150-ml beaker containing 100 ml water and microwave on highest power setting 1 min or until the water starts boiling. Confirm that embedding gel is liquid. CAUTION: Loosen the cap on conical tube with the gel.
16. Slowly add 30 μl embedding gel down the tube wall to cover the pellet from step 14.
17. Without disrupting the pellet, push with the embedding tip to dislodge the mix from the tube wall.
18. Transfer the pellet suspended in embedding gel onto the embedding mold surface, forming a dome-shaped droplet.
19. Incubate droplet at 4° C. for 1 hr.
20. Transfer droplet to sectioning cassette lined with construction paper store in 70% ethanol at 4° C. for up to 1 month.
21. Proceed with paraffin embedding via routine histological processing to prepare paraffin blocks.

### Basic Protocol 4

### Modeling of Reactive Epithelium in Esophageal Organoids

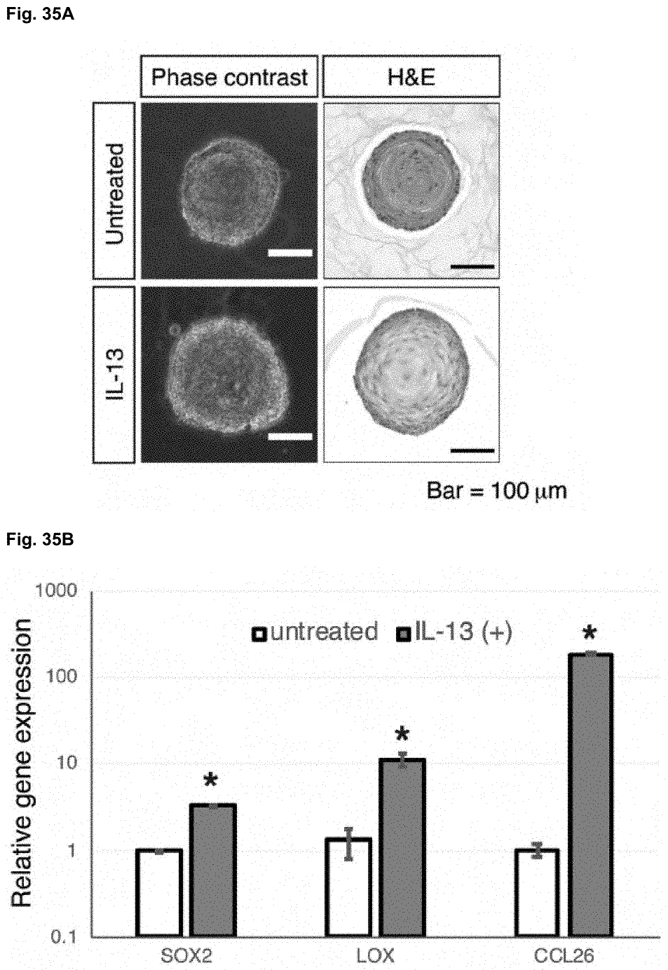

Reactive changes in esophageal epithelium are induced by multiple soluble factors secreted by immune cells and fibroblasts. The function of these factors in disease development can be modeled and evaluated in esophageal organoids. Here we provide an example of such an experiment by treating normal esophageal organoids with interleukin 13 (IL-13) to model reactive changes induced in esophageal epithelium by eosinophils; however, any bioactive compounds (cytokines, antibodies, small molecule inhibitors) can be utilized to model pathologic conditions and/or treatment strategies. Representative images of esophageal organoids treated with IL-13, as well as gene expression profiles of select EoE-relevant genes (Blanchard et al., 2007; Kasagi et al., 2019), can be found in FIGS. 35A-35B.

Additional Materials (see also Basic Protocol 1)
Interleukin 13 (IL-13; R&D Systems, cat. no. 213-ILB-005)
Reconstituted IL-13 (reconstitute lyophilized IL-13 to 100 μg/ml in PBS, aliquot, and store at −80° C.; avoid repeated freeze-thaw cycles)
Growing culture of esophageal organoids (see Basic Protocol 1)
1. Remove spent medium.
2. Dispense 500 μl KSFMC containing 10 ng/ml IL-13.
3. Observe organoid growth under a phase-contrast microscope.
4. Harvest organoids for histology and RNA isolation, as described in Basic Protocol 3.
5. Evaluate changes in morphology and gene expression.

### Support Protocol

### Procurement of Murine Esophageal Epithelial Sheets

This section describes the procedure for isolation of epithelial sheets from murine esophagus. After completion of this protocol, proceed to Basic Protocol 1, step 4 for generation of murine esophageal organoids.

### Additional Materials (See Also Basic Protocol 1)

Mice (e.g., 57BL/6 mice, The Jackson Laboratory, cat. no. 000664)
Gibco amphotericin B, 0.5 μg/ml (Thermo Fischer Scientific, cat. no. 15290018)
HBSS (Corning, cat. no. 21-021-CVR)
CO₂ gas chamber
Sterile dissection-grade scissors (VWR, cat. no. 25870-002)
Sterile forceps (VWR, cat. no. 82027-386)
Sterile iris microdissecting scissors (Carolina Biological Supply, cat. no. 623555)
Petri dish (Thermo Fisher Scientific)

### Dissect and Establish Single-Cell Suspension

1. Sacrifice mice according to your IACUC-approved euthanasia protocol.
2. Use sterile forceps to remove the esophagus and place it in a petri dish containing HBSS.
3. Open esophagus with sterile iris microdissecting scissors in the longitudinal direction; rinse tissue in HBSS.
4. Place opened esophagus in a microcentrifuge tube containing 500 μl dispase and incubate at 37° C. in the thermomixer at 800 rpm for 10 min.
5. Peel epithelium away from the submucosa (and discard submucosa).
6. Proceed with organoid generation as described in Basic Protocol 1, beginning at step 4.

### Commentary

### Background Information

Historically, in vitro analysis of benign disorders of the esophageal epithelium involved stimulation of 2D cultures (Lim et al., 2009; Muir et al., 2013). However, the ability to assess the functional properties of the barrier as well as proliferation and differentiation are limited in flat cultures. To fill this void, organotypic culture (OTC) was adopted from methods of differentiating dermal keratinocytes (Kalabis et al., 2012). In this method, esophageal keratinocytes are seeded on top of a collagen/fibroblast raft. After confluence is reached, epithelia are exposed to an air-liquid interface (ALI) and high calcium concentration in order to induce terminal differentiation. The result is a stratified squamous epithelium with underlying stroma, which mimics in vivo tissue architecture.

More recently, direct methods of evaluating barrier integrity of the esophageal epithelium have been developed capitalizing on the ability of esophageal cells to differentiate when exposed to an ALI (Nguyen et al., 2018; Ruffner et al., 2019; Sherrill et al., 2014). ALI culture involves growing epithelium on a porous membrane. After reaching confluence, the monolayers are exposed to high concentrations of calcium, and medium is then removed from the upper chamber allowing exposure of the cells to air. These methods allow for histologic evaluation of stratified squamous epithelium as well as functional evaluation of mucosal integrity by measuring transepithelial resistance or fluorescein isothiocyanate (FITC) dextran flux to assess permeability.

While ALI and OTC methods allow for evaluation of the esophageal epithelium in 3D, both require a large number of cells grown for multiple passages ex vivo prior to 3D culture development, and performing OTC and ALI cultures with primary patient-derived cells is challenging (Whelan et al., 2018). On the other hand, with the methods of organoid culture demonstrated here, a single biopsy allows for immediate assessment and characterization of the epithelium in 3D within 10 days of procuring the biopsy.

DeWard et al. first described esophageal epithelial organoid cultures from murine tissue (DeWard et al., 2014). Their methods involved utilizing advanced Dulbecco's modified Eagle's medium with multiple growth factors including: Glutamax, B27, p38 kinase inhibitor, EGF, TGFβ inhibitor, R-spondin, Noggin, and Wnt3A. It was found that human esophageal organoids did not grow or stratify in this enriched medium (Kasagi et al., 2018). Instead, a simplified medium of KSFM with additional calcium was utilized that produced a stratified squamous epithelium from both murine and human tissue with almost 100% reliability.

The recent publication demonstrates the use of this technique to recapitulate the unique epithelial changes associated with EoE by stimulation with EoE-relevant cytokines (IL-13, IL-4; Kasagi et al., 2018). The effect of the EoE milieu on Notch signaling was evaluated, and genetic and pharmacologic inhibition of Notch signaling of organoids was utilized to simulate the reactive epithelial changes that occur in vivo. It is expected that future work will utilize organoid technology to evaluate inherent differences in the epithelial tissue from disease state and healthy controls, as well as employ high-throughput drug screening protocols and genetic manipulation.

### Critical Parameters and Troubleshooting

### Biopsy Procurement

Biopsies procured from a normal esophagus tend to result in >85% cell viability. At times those from diseased tissue can contain exudate and dead cells, reducing the overall epithelial viability. In these cases, it is suggested collecting two biopsies per patient to ensure an adequate number of organoids are produced. Similarly, when patients are scoped with a pediatric endoscope due to critical stricture, small biopsy forceps are used and two biopsies may be required.

### Passage of Primary Specimens

It was found that the well described immortalized non-transformed EPC2-hTERT organoids passage indefinitely (Kasagi et al., 2018). Organoids from biopsies do not grow beyond passage four to five. Upon adding supplements Wnt 3A, Noggin/R-spondin, or A83-01 at the time of passage, there was improved OFR; however, it was not indefinite. It is hypothesized that current culture conditions are permissive for keratinocyte progenitors with limited self-renewal capability.

### Day of Stimulus or Harvest

It is critical to consider time points for stimulation and harvest for organoid culture. A critical question to consider is whether OFR, organoid size, differentiation, or proliferation will be evaluated. It is strongly suggested using time course for stimulants, as early stimulation (Days 0 to 4) may affect OFR, whereas later stimulation (Days 5 to 7) may affect proliferation/differentiation.

Similarly, it is critical to harvest cells for evaluation at the same time points. Due to the presence of calcium in the medium, terminal differentiation does occur. Harvesting one experiment on day 11 and another on day 12 may result in vastly different results that are not comparable.

### Understanding Results

### Organoid Formation Rate

The organoids are developed from a single-cell suspension and each organoid arises from a keratinocyte progenitor with self-renewal capability. Thus, the OFR is a quantifiable assessment of the self-renewing epithelial cells in a given population. For instance, we found that organoids from EoE patients and non-EoE controls had similar OFR despite the fact that EoE patients have marked expansion of the basal population. This signifies that despite expansion of the basal population, there is not an increase in the replicative cell population. OFR is calculated as the number of organoids formed, normalized to the number of primary keratinocytes seeded. For example, if 200 organoids were formed after seeding 2,000 cells, OFR would be 0.1 (or 10%). In this Example, typical OFR from normal and EoE patient-derived biopsies is 2%.

### Evaluating Epithelial Differentiation

Careful morphologic evaluation of the organoids provides information regarding epithelial differentiation. Organoids should be measured and average organoid size per well should be assessed at each passage. Methodologies to evaluate differentiation may vary and complementary methods may be utilized. In the recently published work (Kasagi et al., 2018), the organoids were evaluated by an expert pathologist for basaloid cell content and complimented this with immunohistochemistry and flow cytometry analysis for involucrin and CD29, respectively.

### Time Considerations

Procuring the biopsies and making the organoids on Day 0 in the simplified medium takes 2 hours. Having the reagents thawed and ready makes it a more efficient process. Replenishing spent medium and stimulating the organoids with cytokines takes ˜15 min, and harvesting also takes ˜2 hours. The organoids need to be fixed overnight after harvesting for paraffin embedding.

### Example 4

### Generation and Characterization of Patient-Derived Head and Neck, Oral, and Esophageal Cancer Organoids

Generated from endoscopic biopsies or surgically resected tumors, patient-derived organoids (PDO) recapitulate cancer cell heterogeneity within tumors. PDO represent a highly translatable platform for personalized medicine. Grown within 14 days, PDO allow rapid evaluation of therapeutic effects of drugs, both standard of care and molecularly-targeted therapies. PDO serve as an experimental platform to study genetic and environmental factors, as well as signaling pathways, in tumor development and progression that may be variable from patient to patient. This Example describes extensive protocols to generate and characterize esophageal cancer PDO.

Esophageal cancers comprise adenocarcinoma and squamous cell carcinoma, two distinct histologic subtypes. Both are difficult to treat and among the deadliest human malignancies. This Example describes protocols to initiate, grow, passage, and characterize patient-derived organoids (PDO) of esophageal cancers, as well as squamous cell carcinomas of oral/head-and-neck and anal origin. Formed rapidly (<14 days) from a single-cell suspension embedded in basement membrane matrix, esophageal cancer PDO recapitulate the histology of the original tumors. Additionally, this Example provides guidelines for morphological analyses and drug testing coupled with functional assessment of cell response to conventional chemotherapeutics and other pharmacological agents in concert with emerging automated imaging platforms. Predicting drug sensitivity and potential therapy resistance mechanisms in a moderate-to-high throughput manner, esophageal cancer PDO are highly translatable in personalized medicine for customized esophageal cancer treatments. The following protocols are provided in this Example:
Basic Protocol 1: Generation of esophageal cancer PDO
Basic Protocol 2: Propagation and cryopreservation of esophageal cancer PDO
Basic Protocol 3: Imaged-based monitoring of organoid size and growth kinetics
Basic Protocol 4: Harvesting esophageal cancer PDO for histological analyses
Basic Protocol 5: PDO content analysis by flow cytometry
Basic Protocol 6: Evaluation of drug response with determination of the half-inhibitory concentration (IC₅₀)
Support Protocol: Production of RN in HEK293T cell conditioned medium

### Introduction

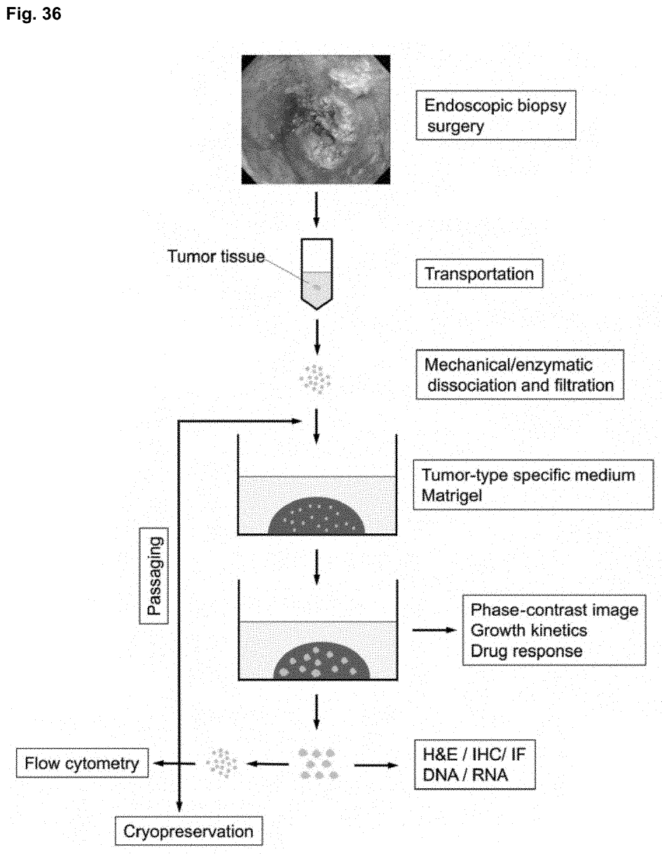

Esophageal cancers comprise esophageal adenocarcinoma (EAC) and esophageal squamous cell carcinoma (ESCC), two distinct histologic subtypes. Both EAC and ESCC are among the deadliest of all human malignancies featuring presentation at late stages, therapy resistance, early recurrence and poor prognosis (Rustgi and El-Serag, 2014). Grown rapidly ex vivo, patient-derived organoids (PDO) recapitulate the original tissue architecture of primary esophageal tumors (Kijima et al., 2019). This Example describes methods to generate and characterize esophageal cancer PDO in terms of growth, morphology and biology. Tissue specimens (diagnostic biopsies or surgically resected tumor tissues) are subjected to enzymatic and mechanical disruption in order to obtain single-cell suspensions, which are embedded in basement membrane matrix (Matrigel®) and cultured in the unique organoid growth medium optimized for distinct histologic tumor types (e.g., adenocarcinoma vs. squamous cell carcinoma). The medium is replaced every other day. Following 14 days of culture, the resulting primary PDO are passaged, cryopreserved or harvested for morphological and functional analyses (FIG. 36).

The harvested organoids can be subjected to a variety of morphological and functional assays including, but not limited to, immunohistochemistry, immunofluorescence, immunoblotting, flow cytometry, quantitative polymerase chain reaction, and RNA sequencing (bulk and single-cell). The conditioned medium from organoid cultures can be used for enzyme-linked immunosorbent assays. Passaged organoids can be tested for conventional and experimental therapeutics in a moderate-to-high throughput manner. Drug treatment of 3D organoids with variable concentrations of therapeutic agents determines their half maximal inhibitory concentration (IC₅₀). Analysis of surviving cells provides insights into the potential drug resistance mechanisms.

Taken together, these methods provide a comprehensive experimental platform to study the molecular mechanisms underlying esophageal cancer cell propagation and drug responses.

### Strategic Planning

Studies need to be carried out as part of Institutional Review Board (IRB)-approved protocols with HIPAA compliance. Esophageal tumor specimens are procured from patients who consented to research biopsies during diagnostic upper endoscopy or surgery (e.g., esophagectomy or endoscopic mucosal resection) by expert gastroenterologists or surgeons at appropriate clinical care facilities with well-trained staff including clinical coordinators. Laboratory personnel should receive laboratory safety training about infectious agents such as human papilloma virus (HPV), hepatitis viruses (HBV, HCV), and human immunodeficiency virus (HIV) that may be potentially present in the patient materials. IRB protocols should be carefully designed to permit investigators access to corresponding patient clinical information such as age, gender, medical history, endoscopic findings, pathology details, therapy received, and patient outcomes.

Fresh tumor specimens need to be transported on wet-ice to a research laboratory for tissue processing and initiation of 3D organoids culture. Laboratory personnel should be notified in advance to schedule organoid culture. To maximize viable cell yield, tissue pieces should be processed as soon as possible. For overnight shipping, samples should be placed in polypropylene tubes (15 ml) filled with basal medium containing penicillin, streptomycin, gentamicin, and amphotericin B to prevent cell culture contamination.

### Basic Protocol 1

### Generation of Esophageal Cancer PDO

A tissue specimen was obtained via diagnostic biopsy or surgery (esophagectomy or endoscopic mucosal resection) and dissociated by enzymatic digestion (dispase and trypsin) and embedded into a single-cell suspension in Matrigel® matrix. PDO are grown in tumor type-specific organoid medium at 37° C. under a controlled atmosphere with 5% CO₂ and 95% relative humidity, resulting in formation of spherical 3D structures representative of the original tumor.

### Materials

Tissue specimen kept at 4° C. (or on wet-ice) in a 15-ml polypropylene tube containing Basal Medium (Table 1)
Hanks' balanced salt solution (HBSS) containing Dispase and Fungizone (HBSS-DF; see recipe in Reagents and Solutions)
Hanks' balanced salt solution (HBSS)-DFCY (see recipe); optional 0.25% Trypsin-EDTA (Invitrogen, cat. no. 25200056); stored at 4° C. until use
Dulbecco's phosphate-buffered saline (DPBS; Thermo Fisher Scientific, cat. no. 14190250)
DNase I (optional) (Sigma-Aldrich, cat. no. 10104159001): Reconstituted to 50 U/ml in sterile DPBS and stored in aliquots at −20° C.
Soybean trypsin inhibitor (STI; see recipe)
Basal medium (Table 1)
0.4% Trypan Blue (Thermo Fisher Scientific, cat. no. T10282)
Matrigel® matrix (see recipe)
Ice
Tumor type-specific organoid medium for ESCC (Table 2)
Tumor type-specific organoid medium for EAC (Table 3)
60-mm cell culture dish (Thermo Fisher Scientific, cat. no. 13-690-081)
Forceps (VWR, cat. no. 82027-386)
Dissecting scissors with 30-mm cutting edge (VWR, cat. no. 25870-002)
1.7-ml microcentrifuge tube (DOT Scientific, cat. no. RN1700-GMT)
Eppendorf ThermoMixer C (Thermo Fisher Scientific, cat. no. 14-285-562PM)
Spectrafuge benchtop mini-centrifuge (Spectrafuge, Labnet, cat. no. C1301) to spin 1.7-ml tubes quickly at room temperature
Serological pipettor (pipette controller, e.g., PORTABLE PIPET-AIDR W/110V charger) (Drummond Scientific, cat. no. 4-000-100)
10-ml disposable plastic pipette (Thermo Fisher Scientific, cat. no. 170356)
1000-, 200-, and 20-μl pipettor (Thermo Fisher Scientific, cat. no. FA10006MTG,
FA10005MTG, and FA10004MTG)
1250-μl barrier pipette tips (GeneMate, cat. no. P-1237-1250)
200-μl barrier pipette tips (GeneMate, cat. no. P1237-200)
20-μl barrier pipette tips (GeneMate, cat. no. P1237-20)
50-ml conical sterile polypropylene centrifuge tube (Thermo Fisher Scientific, cat. no. 12-565-270)
100-μm cell strainer (Corning, cat. no. 431752)
1-ml syringe with a rubber plunger (BD Slip-Tip Tuberculin Syringe without needle, cat. no. 309659)
Sorvall Centrifuge (Sorvall ST 16R, Thermo Fisher Scientific, cat. no. 75004380): Tx-400 Rotor with round buckets (Thermo Fisher Scientific, cat. no. 75003655) for 15-ml conical tubes and 5-ml round-bottom tubes for flow cytometry
Countess™ cell counting chamber slide (Thermo Fisher Scientific, cat. no. C10228)
Countess™ II Automated Cell Counter (Invitrogen, cat. no. AMQAX1000)
24-well plate (Thermo Fisher Scientific, cat. no. 12-556-006)
Eppendorf Refrigerated Centrifuge (Eppendorf 5424R, Thermo Fisher Scientific, cat. no. 5404000014) to spin 1.7-ml tubes in a controlled manner (i.e., time and relative centrifugal force)
CO₂ incubator (e.g., Heracell 150i CO₂ incubator, Thermo Fisher Scientific)
Phase-contrast microscope

TABLE 1
Basal Medium

| | | | Final | |
|---|---|---|---|---|
| Reagent | Volume | | concentration | |
| Advanced DMEM/F12 (Thermo Fisher | 500 | ml | | |
| Scientific, cat. no. 12634028) | | | | |
| GlutaMAX (100×; Thermo Fisher | 5 | ml | 1× | |
| Scientific, cat. no. 35050061) | | | | |
| HEPES (1M; Thermo Fisher Scientific, | 5 | ml | 10 | mM |
| cat. no. 15630080) | | | | |
| Antibiotic-Antimycotic (100×; Thermo | 5 | ml | 1× | |
| Fisher Scientific, cat. no. 15240062) | | | | |
| Gentamicin (50 mg/ml; Thermo Fisher | 50 | μl | 5 | μg/ml |
| Scientific, cat. no. 15750060) | | | | |

TABLE 2
ESCC Organoid Medium (50 ml)

| | | | Final | |
|---|---|---|---|---|
| Reagent | Volume | | concentration | |
| Basal medium (see Table 1) | 47 | ml | | |
| RN conditioned medium (see the Support Protocol) | 1 | ml | 2% | |
| N-2 (100×; Thermo Fisher Scientific, cat. no. 17502048) | 500 | μl | 1× | |
| B-27 (50×; Thermo Fisher Scientific, cat. no. 17504044) | 1 | ml | 1× | |
| N-Acetylcysteine (NAC), 0.5M (Sigma-Aldrich, cat. no. | 100 | μl | 1 | mM |
| A9165), reconstituted in DPBS, filter-sterilized and stored | | | | |
| in aliquots at −20° C. | | | | |
| Recombinant human epidermal growth factor (EGF), | 5 | μl | 50 | ng/ml |
| 500 ng/μl (Peprotech, cat. no. AF-100-15), reconstituted | | | | |
| in basal medium, stored in aliquots at −20° C. | | | | |
| Y-27632 (10 mM; Selleck Chemicals, cat. no. S1049)^{a}; | 50 | μl | 10 | μM^{b} |
| reconstituted in DPBS and stored in aliquots at −20° C. | | | | |
| Gentamicin (50 mg/ml; Thermo Fisher Scientific, cat. no. | 5 | μl | 10 | μg/ml |
| 15750060) | | | | |

| | | | |
|---|---|---|---|
| Antibiotic-Antimycotic (×100; Thermo Fisher Scientific, | 500 | μl | 1× |
| cat. no. 15240062) | | | |

^{a}Only needed when establishing during day 0-day 2.
^{b}Note that basal medium contains 5 μM gentamicin before this supplementation.

TABLE 3
EAC Organoid Medium (50 ml)

| | | | Final | |
|---|---|---|---|---|
| Reagent | Volume | | concentration | |
| Basal medium (see Table 1) | 24 | ml | | |
| L-WRN cell-conditioned medium expressing Wnt-3A, | 24 | ml | 50% | |
| R-Spondin1 and Noggin (WRN), stored at −20° C. | | | | |
| N-2 (100×; Thermo Fisher Scientific, cat. no. 17502048) | 500 | μl | 1× | |
| B-27 (50×; Thermo Fisher Scientific, cat. no. 17504044) | 1 | ml | 1× | |
| N-Acetylcysteine (NAC), 0.5M (Sigma-Aldrich, cat. no. | 100 | μl | 1 | mM |
| A9165), reconstituted in DPBS, filter-sterilized and stored | | | | |
| in aliquots at −20° C. | | | | |
| CHIR99021 (5 mM; Cayman Chemical, cat. no. 13122), | 5 | μl | 0.5 | μM |
| reconstituted in DMSO, stored in aliquots at −20° C. | | | | |
| Recombinant human epidermal growth factor (EGF), | 25 | μl | 250 | ng/ml |
| 500 ng/μL (Peprotech, cat. no. AF-100-15), reconstituted | | | | |
| in basal medium, stored in aliquots at −20° C. | | | | |
| A83-01 (5 mM; Cayman Chemical, cat. no. 9001799), | 5 | μl | 0.5 | μM |
| reconstituted in DMSO, stored in aliquots at −20° C. | | | | |
| SB202190 (10 mM; Selleck Chemicals, cat. no. S1077), | 5 | μl | 1 | μM |
| reconstituted in DMSO, stored in aliquots at −20° C. | | | | |
| Gastrin (1 mM; Sigma-Aldrich, cat. no. G9145), | 5 | μl | 0.1 | μM |
| reconstituted in sterile 0.1% NaOH, stored in aliquots | | | | |
| at −20° C. | | | | |
| Nicotinamide (1M; Sigma-Aldrich, cat. no. N0636), | 1 | ml | 20 | mM |
| reconstituted in DPBS, filter-sterilized and stored in | | | | |
| aliquots at −20° C. | | | | |
| Y-27632 (10 mM; Selleck Chemicals, cat. no. S1049), | 50 | μl | 10 | μM |
| reconstituted in DPBS, stored in aliquots at −20° C. | | | | |
| Gentamicin (50 mg/ml; Thermo Fisher Scientific, cat. no. | 5 | μl | 10 | μM^{b} |
| 15750060) | | | | |
| Antibiotic-Antimycotic (×100; Thermo Fisher Scientific, | 500 | μl | 1× | |
| cat. no. 15240062) | | | | |
| FGF-10^{a} (100 μg/ml; Peprotech, cat. no. 100-26), | 50 | μl | | |
| reconstituted in Basal medium, stored in aliquots at −20° C. | | | | |

^{a}Only added when establishing primary cultures and recovering from frozen stocks
^{b}Note that basal medium contains 5 μM gentamicin before this supplementation.

TABLE 4
HEK293T Medium

| | | Final |
|---|---|---|
| Reagent | Volume | concentration |
| Dulbecco's modified Eagle's medium | 500 ml | |
| (DMEM; Corning, cat. no. MT100133CV) | | |
| Fetal bovine serum (FBS; HyClone, cat. | 50 ml | 10% |
| no. SH3007003) | | |
| Penicillin (100 U/ml)-streptomycin | 5 ml | 1× |
| (100 μg/ml) (100×; Thermo Fisher | | |
| Scientific, cat. no. 15140122) | | |

All reused surgical tools (forceps and dissecting scissors) should be properly cleansed and autoclaved prior to use. Cell culture plasticware and glassware should be sterile and disposable. Water used to dissolve or dilute reagents should be ultrapure (e.g., Milli-Q®) and sterile. Standard equipment and tools for cell culture and cell biology (CO₂ incubator, tissue culture hoods, liquid nitrogen cell storage tank, vacuum aspirator/collection system, centrifuges, electronic pipettors, etc.) are needed. While alternatives are available from multiple vendors, key items used routinely in the laboratories were listed. Use sterile, individually wrapped serological pipettes and barrier pipette tips to minimize microbacterial/fungal contamination to prepare cell culture media (Tables 1-4) and during all cell culture processes described in Basic and Support Protocols.

### Dissociation of Human Esophageal Biopsies to a Single-Cell Suspension

1. Transfer a tumor tissue with sterile forceps into a 60-mm cell culture dish.
2. Mince the tissue to smaller fragments (<1 mm) with sterile dissecting scissors.
3. Transfer minced tissue fragments into a 1.7-ml tube containing 1 ml HBSS-DF.
4. Transfer the tube from step 3 in Thermomixer C to incubate for 10 min at 37° C. with simultaneous mixing at 800 rpm. Optional: Collagenase IV and Y-27632 may be added into HBSS-DF (HBSS-DFCY) with an extended incubation time period for ˜45 min to increase single cell yields.
5. Spin down quickly (˜10 seconds on Spectrafuge) at room temperature.
6. Discard the supernatant using a single-channel 1000-μl pipettor with a 1250-μl tip.
7. Resuspend the pellet with 1 ml of 0.25% trypsin-EDTA and incubate for 10 min at 37° C. with simultaneous mixing at 800 rpm in Thermomixer C. Optional: DNase I may be added in order to degrade DNA released from broken cells to minimize cell aggregates. Add 10 μl of 50 U/ml DNase I into 1 ml of 0.25% trypsin-EDTA to the final concentration of 0.5 U/ml.
8. Filter trypsinized tissue fragments (˜1 ml) from step 7 over a 100-μm strainer into a 50-ml tube containing 8 ml STI. Optional: Residual tissue fragments may be pelleted by spinning down quickly (˜10 seconds on Spectrafuge) at room temperature. Steps 7 and 8 may be repeated to increase single cell yields.
9. Remove a rubber plunger from a 1-ml syringe to use the rubber part of the plunger from a 1-ml syringe to force through the remaining tissue fragments over the strainer.
10. Take ˜8 ml of the filtrate (cell suspension) out from the 50-ml tube using a 10-ml pipette attached to a serological pipettor. Use this filtrate to repeat the wash of the strainer into the identical 50-ml tube. Repeat this three times.
11. Transfer the filtrate into a 15-ml tube and centrifuge for 5 min at 188×g (1000 rpm on Sorvall ST 16R), 4° C.
12. Discard the supernatant first by aspiration leaving the last ˜1 ml, which should be removed using a 1000-μl pipettor with a 1250-μl tip so as not to disturb the pellet. 13. Resuspend the cell pellet in 1 ml basal medium with gentle pipetting. 14. Take 10 μl of the cell suspension from step 13 and mix with 10 μl of 0.4% Trypan Blue (to stain dead cells) and load onto a cell counting chamber slide.
15. Determine cell density and cell viability (via Trypan Blue exclusion test) with Countess™ II Automated Cell Counter.

### PDO Initiation and Growth in 24-Well Plates

In general, 2×10⁴ cells will be seeded per well to initiate PDO in 24-well plates.
16. Thaw and keep Matrigel® on ice.
17. Pre-warm a 24-well plate at 37° C. and pre-warm organoid medium in 37° C. water bath.
18. Use 6 wells to generate sufficient number of organoids for both initial propagation in a subsequent passage (3 wells) and morphological analysis (3 wells). Prepare 1.4×10⁵ cells (=7 wells×2×10⁴ cells/well for 6 wells plus an extra well). Take the necessary volume (ml) of cell suspension according to the following formula: 1.4×10⁵ [cell number needed]+[cell density (/ml) from step 15]
19. Transfer 1.4×10⁵ cells into a 1.7-ml tube. 20. Centrifuge for 3 min at 500×g (2300 rpm on Eppendorf 5424R), room temperature, to pellet the cells.
21. Resuspend the cells in 350 μl ice-cold Matrigel [50 μl/well×(6+1) wells].
22. Dispense 50 μl each of cells-suspended Matrigel into each well of 24-well plate.
23. Place the 24-well plate in a CO₂ incubator (37° C.) for 30 min to allow the Matrigel to solidify.
24. Add into each well 500 μl tumor-type specific organoid medium (Tables 2 and 3) for ESCC or EAC according to clinical diagnosis and pathology report of the original tumor.
25. Refresh the organoid medium every 2-3 days. To remove spent medium, use a 1000-μl pipettor or aspirate very carefully so as not to disturb the Matrigel.
26. Monitor contamination and organoid growth under a phase-contrast microscope (see Basic Protocol 3).
27. Grow organoids for up to 10-14 days to be passaged or harvested. If spherical structures (organoids) emerge but grow slowly, extend the culture period by additional 7-14 days.

### Basic Protocol 2

### Propagation and Cryopreservation of Esophageal Cancer PDO

Once established, esophageal cancer cells can be isolated from the primary PDO by enzymatic dissociation to seed subsequent passage (i.e., sub-culture) in order to propagate further for histological analyses in Basic Protocol 4 and flow cytometry in Basic Protocol 5. PDO may be sub-cultured in 96-well plates for experiments such as drug treatment in Basic Protocol 6. Additionally, isolated esophageal cancer cells can be cryopreserved for long-term storage.

Additional Materials (also see Basic Protocol 1)
Growing PDO (see Basic Protocol 1)
Dimethyl sulfoxide (DMSO; Sigma-Aldrich, cat. no. D4540)
Freezing medium (see recipe)
ISOTEMP 220 water bath (37° C.; Thermo Fisher Scientific, cat. no. 15-462-20Q)
5-ml Falcon round-bottom tube with a 35-μm cell strainer cap (BD Bioscience, cat. no. 352235)
96-well plate (Thermo Fisher Scientific, cat. no. 12-556-008)
Nalgene general long-term storage cryogenic tubes (Thermo Fisher Scientific, cat. no. 03-337-7D)
Cool cell container (Corning, cat. no. 432002)

### Disintegration of Mature PDO to Prepare a Single Cell Suspension for a Subsequent Sub-Culture

PDO growing in 3 wells (Basic Protocol 1) will be used for sub-culture.
1. Remove culture medium carefully using a 1000-μl pipettor with a 1250-μl tip so as not to disturb the Matrigel containing growing mature organoids in each well. Aspiration is not recommended at this stage because Matrigel becomes increasingly fragile as PDO grow.
2. Add 500 μl of cold DPBS into each well and disrupt the Matrigel mechanically into small fragments by pipetting up and down 3 times through a 1250-μl pipette tip.
3. Combine PDO-containing Matrigel from 3 wells and transfer into a 1.7-ml tube.
4. Centrifuge for 3 min at 500×g (2300 rpm on Eppendorf 5424R), room temperature, to pellet the Matrigel fragments.
5. Discard the supernatant using a 1000-μl pipettor.
6. Resuspend the Matrigel fragments with 1 ml Trypsin-EDTA OPTIONAL: If desired, supplement with DNase I as described in Basic Protocol 1, step 7.
7. Incubate for 10 min at 37° C. with simultaneous mixing at 800 rpm in Thermomixer C.
8. To disintegrate organoid structures further, add 3-4 strokes of pipetting using a 1000-μl pipettor through a 1250-μl pipette tip.
9. Filter the cell suspension from step 8 over a 35-μm cell strainer cap into a 5-ml Falcon round-bottom tube containing 3 ml STI.
10. Centrifuge for 5 min at 188×g (1000 rpm on Sorvall ST 16R), 4° C.
11. Remove the supernatant by aspiration.
12. Resuspend the cell pellet in 1000 μl basal medium.
13. Determine cell density and viability as in Basic Protocol 1 steps 14-15.

### Passaging PDO in 24-Well Plates

14. To passage PDO in 24-well plates, generally, seed 2×10⁴ live cells per well in accordance with Basic Protocol 1 steps 16-27.

### Passaging PDO in 96-Well Plates

In general, 2-5×103 cells per well will be seeded in 96-well plates.
15. Thaw Matrigel as in Basic Protocol 1 step 16 and keep on ice until use.
16. Pre-warm a 96-well plate at 37° C. in cell culture incubator.
17. To seed 2×10³ cells per well for treatment with a drug (e.g., 5FU) at various concentrations (e.g., 10⁻³, 10⁻², 10⁻¹, 1, 10, 100, 1000 μM) and vehicle (e.g., DMSO for 5FU) in triplicate, prepare 6×10⁴ cells [=30 wells×2×10³ cells/well for 24 wells (3 wells×8 for 5FU and DMSO) and plus extra 6 wells]. Take a necessary volume (ml) of cell suspension according to the following formula: 6×10⁴ [cell number needed]+[cell density (/ml) from Basic Protocol 2 step 13]
18. Transfer 6×10⁴ cells into a 1.7-ml tube.
19. Centrifuge for 3 min at 500×g (2300 rpm on Eppendorf 5424R), room temperature, to pellet the cells.
20. Resuspend the cells in 150 μl ice-cold Matrigel [5 μl/well×(30 wells)].
21. Dispense 5 μl of cells suspended in Matrigel into each well of a 96-well plate.
22. Place the 96-well plate in a CO₂ incubator (37° C.) for 30 min to allow the Matrigel to solidify.
23. Dispense 100 μl tumor-type specific organoid medium into each well (Tables 2 and 3) for ESCC or EAC according to clinical diagnosis and pathology report of the original tumor.
24. Refresh the organoid medium every 2-3 days. Remove spent medium by aspiration. Use a 200-μl pipettor to replenish with 100 μl medium.
25. Monitor contamination and organoid growth under a phase-contrast microscope (see Basic Protocol 3).
26. Grow organoids for up to 7-8 days until they reach 70-100 μm in dimeter for experiments (e.g., drug treatment, Basic Protocol 6).

### Cryopreservation

27. To make three vials of frozen stock, take 3×10⁵ live cells from step 12. Determine the necessary volume of the cell suspension based on the viable cell count in step 13 and transfer the cells into a 1.7-ml tube.
28. Centrifuge for 3 min at 500×g (2300 rpm on Eppendorf 5424R), room temperature.
29. Remove the supernatant using a 1000-μl pipettor with a 1250-μl tip.
30. Resuspend the cell pellets in 1 ml freezing medium (to the final density at 3×10⁵/ml).
31. Dispense 330-μl aliquots into three fresh cryogenic vials.
32. Add 670 μl fresh freezing medium into each cryogenic vial.
33. Freeze each vial in a freezing container overnight at −80° C.
34. Transfer the frozen vials into a liquid nitrogen cell storage tank.
   Recovery after Cryopreservation
35. Thaw Matrigel as described in Basic Protocol 1, step 16 and keep on ice.
36. Pre-warm a 24-well plate at 37° C.
37. Thaw the cryogenic vial (step 34) in a 37° C. water-bath for 30-45 seconds.
38. Transfer the cell suspension into a 1.7-ml tube.
39. Centrifuge for 3 min at 500×g (2300 rpm on Eppendorf 5424R), room temperature, and remove the supernatant using a 1000-μl pipettor.
40. Resuspend the cell pellet in 1 ml DPBS.
41. Centrifuge for 3 min at 500×g (2300 rpm on Eppendorf 5424R), room temperature, and remove the supernatant using a 1000-μl pipettor.
42. Resuspend the cell pellet in 1 ml basal medium, determine cell density and viability as in Basic Protocol 1, steps 13-15.
43. Centrifuge for 3 min at 500×g (2300 rpm on Eppendorf 5424R), room temperature, and remove the supernatant using a 1000-μl pipettor.
44. Resuspend the cells in Matrigel and seed cells (2×10⁴ cells/well) and proceed with organoid culture as described in Basic Protocol 1 steps 17-27. OPTIONAL: For EAC PDO, supplementation of 100 ng/ml FGF-10 (Table 3) in the first week dramatically increases recovery after cryopreservation.

### Basic Protocol 3

### Imaged-Based Monitoring of Organoid Size and Growth Kinetics

To evaluate organoid growth, 3D organoid structures were monitored during culture, and their size is documented by a conventional phase-contrast inverted microscope or highthroughput cell imaging instruments (e.g., Celigo Image Cytometer, Nexcelom Bioscience) with the capacity of automated multi-well rapid imaging and quantitative analysis of organoid size, number and structure.

Additional Materials (also see Basic Protocols 1 and 2)
Inverted microscope with imaging capacity such as Nikon Eclipse E600 microscope (Nikon, Tokyo, Japan) with a camera, EVOS FL Cell Imaging System (Thermo Fisher scientific) or Celigo imaging cytometer (Nexcelom Bioscience)
ImageJ software (NIH)
GraphPad 7.0 (Prism) or SigmaPlot (Systat) software to generate a growth curve

### Evaluating PDO Growth

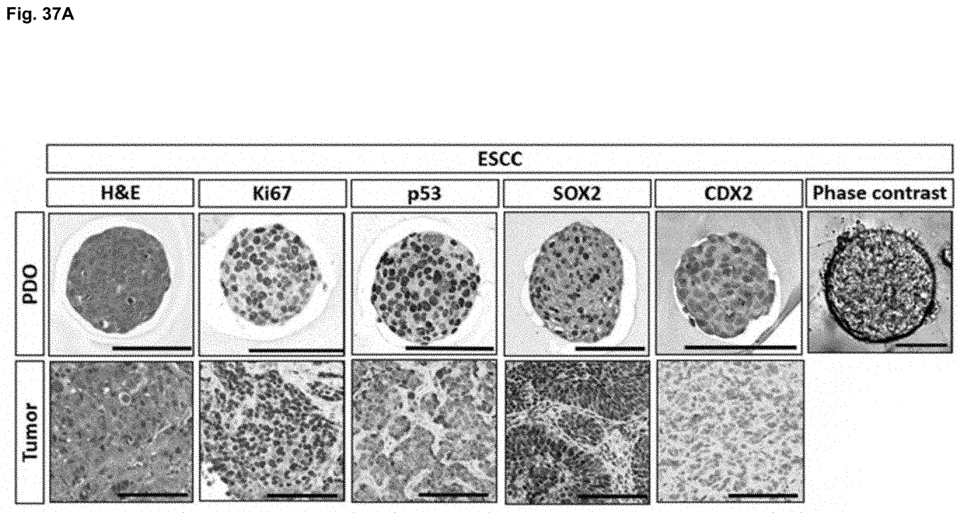

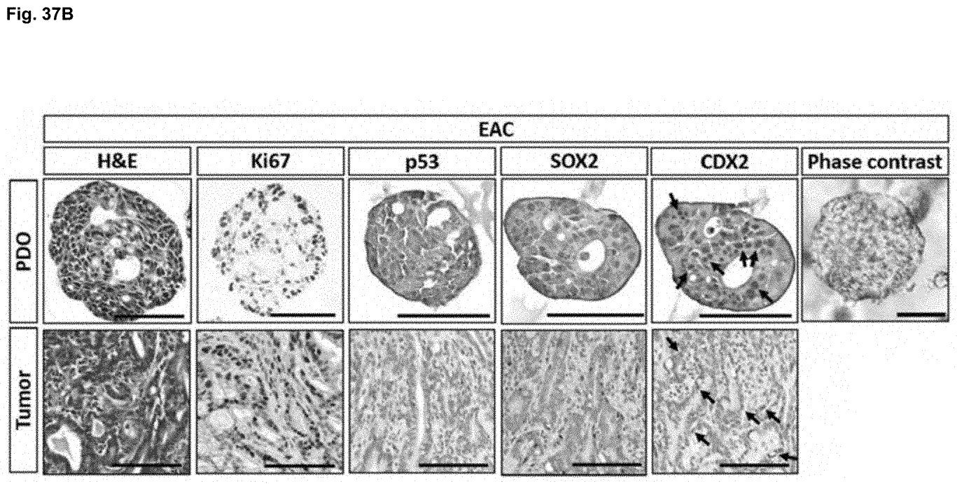

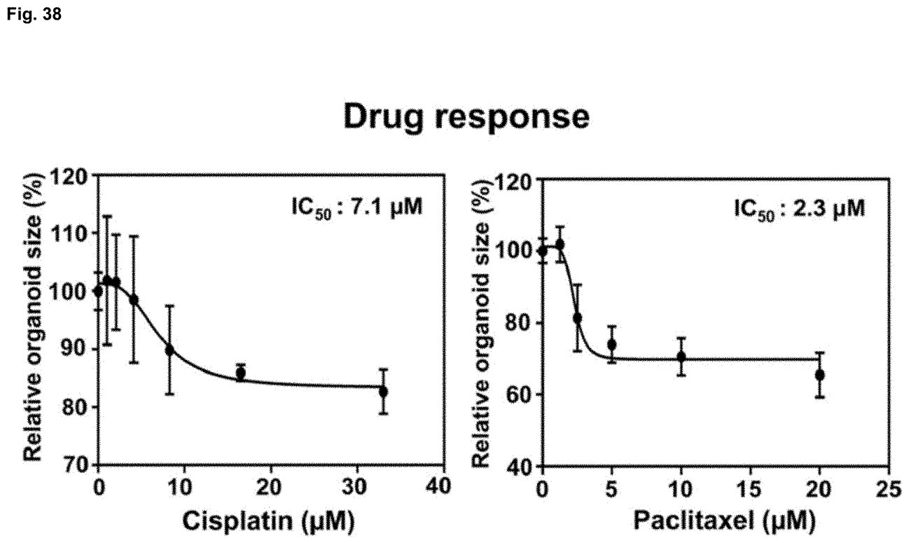

1. Grow organoids in 24-well or 96-well plates (Basic Protocols 1 and 2). Use at least three (3) wells per condition as biological replicates.
2. Acquire phase-contrast images (FIGS. 37A-37B) manually via a conventional microscopy with a camera. Use ImageJ software to measure the diameter of at least 5 organoids per well. Alternatively, Celigo imaging cytometer can not only acquire phase-contrast images but calculate mean organoid area for all organoids imaged. Such assays can be performed in conjunction with drug treatment (FIG. 38) (Basic Protocol 6).
3. Repeat step 2 every other day.
4. Generate growth curves by plotting organoid diameter or area at each time point.

### Basic Protocol 4

### Harvesting Esophageal Cancer PDO for Histologicalanalyses

Histological evaluation is an essential step in ensuring that PDO recapitulate the original tumor morphologically. Herein, we provide a protocol for fixation and embedding of PDO in paraffin, allowing for a long-term storage and histological analyses including hematoxylin-eosin staining, immunohistochemistry, and immunofluorescence.

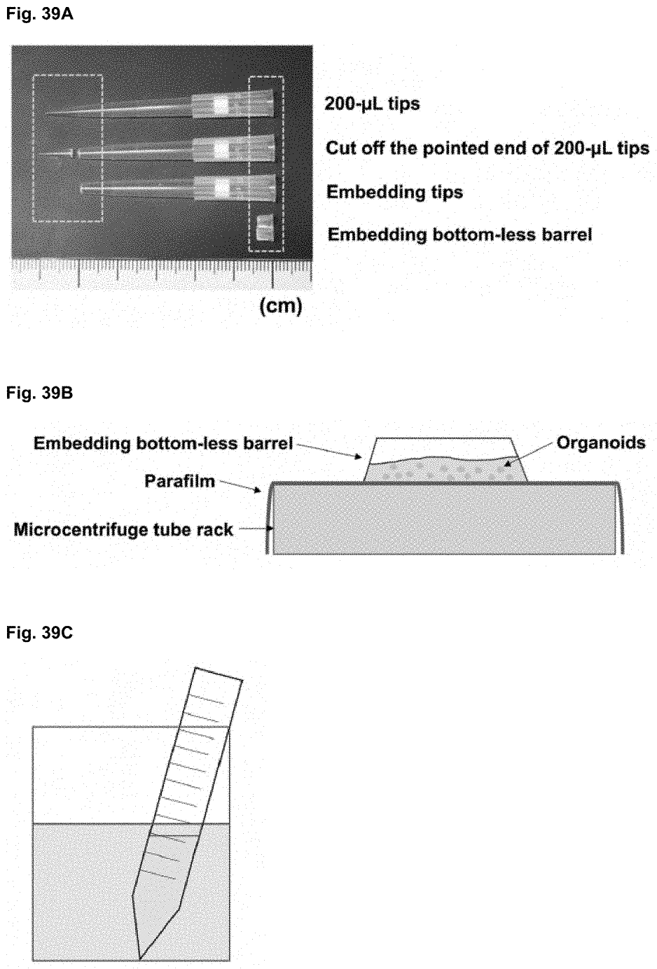

Additional Materials (also see Basic Protocols 1 and 2)
PDO (live organoids in culture) (see Basic Protocol 1)
Paraformaldehyde (PFA), powder, 95% (Sigma-Aldrich, cat. no. 158127-500G)
Embedding gel (see recipe)
Ethanol 200 Proof (EtOH, Thermo Fisher Scientific, cat. no. 22-032-601)
HBSS-D: HBSS containing Dispase (HBSS-D; see recipe)
Kimble KIMAX griffin 150-ml beaker (Thermo Fisher Scientific, cat. no. 02-539J)
Embedding tip (FIG. 39A): a 200-μl pipette tip modified to have a wide opening [use clean dissecting scissors (VWR, cat. no. 25870-002) and cut off ˜9 mm of the pointed end of 200-μl tips to make their opening wider).
Embedding bottom-less barrel (FIG. 39A): The pipettor connecting part of 200-μl pipette tip will be utilized as an embedding cylinder (a bottom-less barrel); use clean dissecting scissors to cut out the proximal ˜4 mm of the cylinder part (i.e., the broader open end) of 200-μl tips
Parafilm M wrapping film (Thermo Fisher Scientific, cat. no. S37440)
Embedding rack
Thermo Scientific™ Shandon™ Sponge (rectangular sponges to hold specimen in cassette, 25×31 mm) (Thermo Fisher Scientific, cat. no. 84-53)
Fisher-brand tissue path IV tissue cassettes (Thermo Fisher Scientific, cat. no. 22-272416)
Microcentrifuge tube rack (5×16 holes/rack) (Southern Labware, cat. no. 0061)

### Fixation of 3D Organoid Cultures

PDO growing in 3 wells (Basic Protocol 1) will be used for histological analyses.
1. Remove culture medium carefully as in Basic Protocol 2, step 1.
2. Add 500 μl cold DPBS into each well, dislodge and disrupt the Matrigel mechanically by pipetting up and down 3-4 times with a 1000-μl pipettor with a 1250-μl tip.
3. Combine the fragmented Matrigel from 3 wells and transfer into a 1.7-ml tube.
4. Centrifuge for 3 min at 500×g (2300 rpm on Eppendorf 5424R), room temperature, to pellet the Matrigel fragments.
5. Remove the supernatant using a 1000-μl pipettor with a 1250-μl tip.
6. Add 1 ml DPBS to dissociate the pellet by pipetting up and down 3-4 times with a 1000-μl pipettor through a 1250-μl tip.
7. Centrifuge for 3 min at 500×g (2300 rpm on Eppendorf 5424R), room temperature, to pellet the Matrigel fragments.
8. Remove the supernatant using a 1000-μl pipettor with a 1250-μl tip.
9. Resuspend the pellet in 500 μl of 4% PFA using a 1000-μl pipettor with a 1250-μl tip.
10. Incubate >2-6 hours or overnight at 4° C.
11. Centrifuge for 3 min at 500×g (2300 rpm on Eppendorf 5424R), room temperature.
12. Remove the supernatant using a 1000-μl pipettor with a 1250-μl tip.
13. Add 1 ml DPBS to wash the pellet by pipetting up and down 3-4 times with a 1000-μl pipettor through a 1250-μl tip.
14. Centrifuge for 3 min at 500×g (2300 rpm on Eppendorf 5424R), room temperature.
15. Remove the supernatant carefully using a 1000-μl pipettor with a 1250-μl tip.
16. Remove as much as possible of the remnant trace volume of the supernatant using a 200-μl pipettor with a 200-μl tip.
17. Proceed to embedding. Alternatively, the cell suspension (step 14) may be stored up to 1 week at 4° C. prior to embedding.

### Embedding and Preparation of Paraffin Tissue Blocks

18. Prepare embedding tips (FIG. 39A) and set up a microcentrifuge tube rack (FIG. 39B) covered with Parafilm M over the top used in step 21.
19. Place the embedding gel (5 ml in a 15-ml tube) in a 150-ml beaker containing ˜100 ml tap water (FIG. 39C). Remove/loosen the cap! This is important for safety to use a microwave in the following step.
20. Microwave at the highest power level for 1 min until the water starts boiling.
21. Confirm that the embedding gel has been liquefied and leave for 2-3 min.
22. Resuspend the organoid pellet in 50 μl liquefied embedding gel using an embedding tip on a 200-μl pipettor (FIG. 39A). Transfer immediately and cast into an embedding bottom-less barrel placed on the Parafilm-covered embedding rack (FIG. 39B). NOTE: The organoid pellet should be minimally dispersed in embedding gel upon pipetting. This will maximize the number of individual organoid structures visible per resulting paraffin section on a glass microscope slide. To this end, detach the packed organoid pellet from the bottom of the 1.7-ml tube (Basic Protocol 4, step 17) using a toothpick or a regular 10 μl tip (without being attached to a pipettor). Then, add 50 μl embedding gel and transfer the minimally disturbed organoid pellet into the embedding barrel using an embedding tip on a 200-μl pipettor.
23. Transfer the embedding rack to 4° C. and let the gel solidify for at least 30 min.
24. Use a fresh embedding tip to gently push out the solidified gel containing embedded organoids onto to sponge placed within a tissue cassette.
25. Place the tissue cassette into 70% ethanol and store at 4° C. until embedding in paraffin via routine histological processing to prepare paraffin blocks.

### Basic Protocol 5

### PDO Content Analysis by Flow Cytometry

Single cell-derived PDO recapitulate intratumoral cell heterogeneity. Besides morphology (Basic Protocol 4), PDO content can be characterized by flow cytometry (e.g., cell surface markers). Such analysis can be done in conjunction with pharmacological treatments to explore unique signaling pathways or therapy resistance mechanisms associated with unique cell populations within PDO. Fluorescence-labeled antibodies, dyes and probes can be utilized to detect a variety of cellular antigens and molecular targets. This Example describes a protocol to determine cell surface CD44 expression as an example of this approach. CD44 is a glycoprotein implicated in the pathogenesis of esophageal cancer (Kinugasa et al., 2015; Natsuizaka et al., 2017; Whelan et al., 2017). Note that antibody titers, selection of fluorochromes, and other assay conditions are variable, requiring optimization for each molecule of interest.

Additional Materials (also see Basic Protocols 1 & 2)
Fluorescence-activated cell sorting (FACS) buffer (see recipe)
APC mouse anti-human CD44 (clone G44-26, BD Biosciences, cat. no. 559942)
4′,6-Diamidino-2-Phenylindole, Dihydrochloride (DAPI; see recipe)
Vortex-Genie2 (Scientific Industries, cat. no. SI-T236)
Aluminum Foil Roll (Thermo Fisher Scientific, cat. no. 01-213-105)
FACSCalibur or LSR II cytometers (BD Biosciences)
FlowJo software (Tree Star)
MilliporeSigma™ Steriflip™ Sterile Disposable Vacuum Filter Units (50-ml tube with a filter with 0.22-μm pore) (Fisher Scientific, cat. no. SE1M179M6)

### Flow Cytometry Analysis of PDO

1. Dissociate organoids grown in a 24-well plate to prepare a single cell suspension and determine cell density as described in Basic Protocol 2 steps 1-12.
2. Transfer 2×10⁵-1×10⁶ cells to a 5-ml Falcon round-bottom tube.
3. Add 4 ml FACS buffer and centrifuge for 5 min at 188×g (1000 rpm on Sorvall ST 16R), 4° C.
4. Discard the supernatant by aspiration.
5. Add 100 μl FACS buffer containing 5 μl conjugated anti-CD44 antibody in cell suspension (1:20, pre-optimized titer) in a 5-ml Falcon round-bottom tube.
6. Mix vigorously by vortexing at an analog speed setting of 8 (max speed) for a few seconds.
7. Incubate the reaction tubes on ice for 30 min (optimized for anti-CD44 antibody binding), protected from light by covering with aluminum foil.
8. Add 4 ml FACS buffer to wash the cells by centrifuging for 5 min at 188×g (1000 rpm on Sorvall ST 16R), 4° C.
9. Discard the supernatant by aspiration and resuspend cells in 500 μl FACS buffer containing 1 μl DAPI, a DNA staining dye to determine dead cells, and mix well as in step 6.
10. Analyze the DAPI-negative cells for CD44 expression using a flow cytometer and FlowJo software (Whelan et al., 2017). To define cells with CD44 expression, a gate set in unstained cells (control) is applied.

### Basic Protocol 6

Evaluation of Drug Response with Determination of the Half-Inhibitory Concentration (IC₅₀)

One of the major goals in PDO translation is to serve as a potential guide to assist clinical decision making by physicians and surgeons in personalized/precision medicine where customized therapeutics are provided following molecular characterization of cancer cells in the original tumors. To this end, PDO need to be tested for multiple drugs in standard of care and molecularly targeted agents (e.g., small molecule inhibitors and antibodies) in a time-sensitive manner. Drug treatment of PDO can be performed in 96-well plates containing established PDO with a broad range of drug concentrations. PDO response to drugs can be evaluated via numerous cell viability assays based upon cellular functions (e.g., ATP production and other mitochondrial activities such as formazan formation in the WST-1 reagent) and cell membrane integrity (e.g., membrane-permeating fluorescent dyes such as Calcein-AM). This Example describes utilization of the CellTiter-Glo® 3D cell viability assay.

### Materials

96-well plate with mature PDO grown for 7-8 days until they reach ˜70-100 μm in diameter (see Basic Protocol 2)
Organoid medium containing drugs (see recipe)
CellTiter-Glo 3D cell viability assay (G9681, Promega): Thaw CellTiter-Glo 3D reagent overnight at 4° C. and equilibrate the CellTiter-Glo 3D reagent to room temperature prior to use
Belly Dancer Shaker (IBI Scientific, cat. no. Z768502-1EA)
Microplate Reader for detecting luminescence (e.g., GloMax® Discover Microplate Reader Promega, cat. no. GM3000)
GraphPad Prism 7.0
   NOTE: White plates such as Nunc™ Nunclon Multi-Dishes, 96 well Plate with Lid, Color White (Thermo Fisher Scientific, cat. no. 165306) are recommended for luminescence-based CellTiter-Glo® 3D cell viability assay to prevent a signal interference from neighboring wells.

### Drug Treatment

1. Remove the spent culture medium by aspiration.
2. Dispense 100 μl of organoid medium containing drugs per well at the range of final concentrations to be tested.
3. Incubate organoids with drugs for 72 hours at 37° C. under a controlled atmosphere with 5% CO₂ and 95% relative humidity under standard organoid growth conditions (see Basic Protocol 1).

### Cell Viability Assay

4. Remove drug-containing medium from each well by aspiration and replace with 100 μl volume of a 1:1 cocktail of CellTiter-Glo 3D and basal medium. Add the cocktail into 3 wells without organoids to measure the background luminescence level. Mix 50 μl CellTiter-Glo 3D regent and 50 μl basal medium (1:1) (100 μl per well).
5. Mix the contents vigorously using the Berry Dancer shaker at speed 4 for 5 min to induce cell lysis.
6. Incubate the plate at room temperature for 25 min.
7. Measure the luminescence by GloMax-Multi+ Microplate Multimode Reader.
8. Generate dose response curves using GraphPad Prism 7.0, the least squares fit (ordinary) with a variable slope (four parameters).

### Support Protocol

### Production of RN in HEK293T Cell Conditioned Medium

Organoid culture media require developmental niche factors including R-spondin1 (R) and Noggin (N). Such factors can be harvested as cell culture conditioned media (Miyoshi and Stappenbeck, 2013), providing a more affordable alternative to commercially available recombinant proteins. Herein, we describe a protocol to produce and harvest conditioned media expressing highly active R and N concurrently (thereby, designated as RN) via lentivirus-mediated transduction of HEK293T cells. The produced RN in conditioned media can be validated in murine small-intestinal organoid formation assays (Sato et al., 2011).

### Preparation of Conditioned Medium

To generate conditioned medium containing high RN activity, high-titer lentivirus-expressing RN can be produced by transient transfection of HEK293T cells. The resulting high-titer virus-containing HEK293T cell conditioned medium is used to infect HEK293T cells to produce RN that will be harvested as a conditioned medium from virus-infected HEK293T cells.

CAUTION: Biosafety level (BL)-2 or enhanced BL-2 is appropriate to perform lentivirus production and infection experiments. The produced virus, albeit replication incompetent, can infect human cells. Treat plasticware such as pipettes, syringe discs, glass Pasteur pipettes, suction lines with Bleach to kill the infectious virus in every step following transfection. For example, make sure to decontaminate a glass Pasteur pipette that was used to change medium containing lentivirus prior to disposal.

### Materials

HEK239T cells (ATCC, cat. no. CRL-3216)
HEK 293T medium (see Table 4), stored at 4° C.
DPBS
Trypsin
Lipofectamine 2000 reagent (life Sciences, cat. no. 11668019), stored at 4° C.
Opti-MEM reduced serum medium (Thermo Fisher Scientific, cat. no. 31985070), stored at 4° C.
pCMVR8.74, 2nd generation lentiviral packaging plasmid (Addgene, cat. no. 22036)
VSV.G (Addgene, cat. no. 14888), mammalian expression plasmid to express VSV-G envelope
pG-N+RIP (unpublished, Rustgi Lab), lentiviral vector expressing RSpondin1 and Noggin
Polybrene (EMD Millipore, cat. no. TR-1003-G)
Puromycin (GoldBio, cat. no. P-600-100)
Bleach (6% sodium hypochlorite, Clorox) (See CAUTION above)
175-cm² tissue culture flask (Fisher Scientific, cat. no. 159910)
100-mm tissue culture dish (Fisher Scientific, cat. no. 130182)
37° C., 5% CO₂ incubator
PES Syringe filter (0.45 μm, 30 mm, Sterile, CellTreat Scientific Products, cat. no. 229749)
150-mm tissue culture dish (Fisher Scientific, cat. no. 130183)
50-ml conical tube (Fisher Scientific, cat. no. 12-565-270)
Rapid-flow vacuum filter unit (Fisher Scientific, cat. no. 566-0020)
10-ml syringe (BD, cat. no. 0309604)
1. Grow HEK293T cells in a large scale using a 75-cm² tissue culture flask, containing 30 ml of HEK 293T medium in a large scale using a 175-cm² tissue culture flask, containing 30 ml of at 5% CO₂ and at 37° C. under >95% humidity. Wash the cells with ˜20 ml DPBS, trypsinize, and count cells via standard cell culture procedures. Note that a mechanical force easily detaches HEK 293T cells from plastic plates, and thus it is important to handle cells gently during DPBS-wash and trypsinization. It takes less than 30 seconds for cells to detach from plates at room temperature when trypsinized. Washing once with DPBS before adding trypsin-EDTA is necessary because FBS in HEK 293T medium may block trypsin activity.
2. Seed 6×10⁶ HEK293T cells in a 100-mm dish and grow for 48-72 hours to 80-90% confluency in the following day.
3. Mix 40 μl Lipofectamine 2000 in 360 μl of Opti-MEM.
4. Incubate for 5 min at room temperature.
5. Add DNA (10 μg pG-N+RIP, 6.5 μg pCMVdR8.74, 3.5 μg VSV.G) in 400 μl of Opti-MEM.
6. Combine Opti-MEM containing Lipofectamine 2000 (step 3) and Opti-MEM containing DNA (step 5).
7. Incubate for 30 min at room temperature.
8. Add 5 ml Opti-MEM into the Opti-MEM-Lipofectamine-DNA cocktail.
9. Remove spent medium from HEK293T cell culture by aspiration and replace by the Lipofectamine-DNA-Opti-MEM mix.
10. Incubate for 4 hours at 37° C.
11. Remove the medium and replace with 7 ml fresh HEK293T medium.
12. Incubate the cells for 48-72 hours at 5% CO₂ and at 37° C. under >95% humidity.
13. Harvest the virus at 48 hours as conditioned medium and replenish the HEK293T medium.
14. Filter the conditioned medium using 0.45-μm syringe filter attached to a 10-ml syringe. Best used immediately, or the virus can be stored up to year at −80° C.
15. Harvest the virus at 72 hours as conditioned medium and replenish the culture with HEK293T medium.
16. Filter the conditioned medium using 0.45-μm syringe filter. Best used immediately, or the virus can be stored up to 1 month at −80° C.

### Lentivirus-Mediated Transduction of HEK293T Cells to Produce RN-Conditioned Medium

17. Plate HEK293T cells in two 100-mm dishes to ˜80%-90% the following day (one dish will be transfected, and the other will be used as control for Puromycin selection). Replace the culture medium with 7 ml virus supplemented with Polybrene (3.5 μl of 10 mg/ml stock). Incubate for 4 hours at 37° C. as in step 10.
18. Add 3 ml HEK293T medium and incubate for 4 hours at 37° C. as in step 17.
19. Replace virus-containing HEK293T medium from step 13 (or step 15).
20. Incubate for 24 hours at 37° C.
21. Add puromycin to the final concentration of 2 μg/ml and continue until cells from the control plate without virus infection are all dead.
22. Wash the cells with DPBS twice.
23. Count cells as in steps 14-15 of the Basic Protocol 1 and seed 5×10⁶ cells into as many 150-mm dishes as possible in HEK293T medium without puromycin.
24. Collect the medium 24, 48, and 72 hours later (store at −80° C. until the last harvest).
25. Combine all conditioned media harvested and filter-sterilize utilizing a rapid-flow vacuum filter unit.
26. Dispense into 50-ml tubes and store up to 1 year at −80° C.
27. After thawing a 50-ml tube, dispense into 1-ml aliquots and store at −20° C.
28. Test RN-conditioned medium in murine intestinal organoid formation assays by comparing dilutions from 1%-25% conditioned medium to medium containing defined recombinant growth factors and chose a final concentration based upon similar growth rates and morphology over the course of 7 days (Sato et al., 2011).

### Reagents and Solutions

Collagenase IV, 200 mg/ml

Dissolve 1 g Collagenase IV (Thermo Fisher Scientific, cat. no. 17104019) into 5 ml HBSS to make a 200 mg/ml stock solution, dispense into 1-ml aliquots, and store at −20° C. Thaw in water bath at 37° C. for 1 min prior to use.

### 4′,6-Diamidino-2-Phenylindole, Dihydrochloride (DAPI)

Dissolve 10 mg DAPI (FluoroPure grade; Thermo Fisher Scientific, cat. no. D21490) into 2 ml water (5 mg/ml) and dilute further to 0.05 mg/ml (50 μg/ml) as a stock solution, dispense into 1-ml aliquots, and store up to 6 months at −20° C. Thaw in water bath at 37° C. for 1 min prior to use. Protect from light.

### Dispase, 50 U/ml

Store undiluted dispase (Corning, cat. no. 354235) aliquots (1 ml) up to 3 months at −20° C. Thaw in water bath at 37° C. for 1 min prior to use.

### Embedding Gel

To prepare 50 ml of 2% (weight/volume) Bacto-Agar-2.5% (weight/volume) gelatin gel, resuspend 1 g of Bacto-agar (Becton Dickinson, cat. no. 214010) and 1.25 g of gelatin (Fisher Scientific, cat. no. G7-500) in 50 ml water in a 150-ml beaker. Swirl the suspension and let it sit for 30-60 min at room temperature. Autoclave for 20 min at 121° C. and dispense into 5-ml aliquots in 15-ml conical tubes. Store up to 6 months at room temperature.

### Fluorescence-Activated Cell Sorting (FACS) Buffer

Dissolve 0.5 g bovine serum albumin (BSA; Sigma-Aldrich. cat no. A7906-100G) in 50 ml DPBS, filter-sterilize using a 0.22-μm filter (MilliporeSigma Steriflip Sterile Disposable Vacuum Filter Units), and store at 4° C.

### Freezing Medium

Mix 9.0 ml fetal bovine serum (FBS; HyClone, cat. no. SH30071.03) and 1.0 ml dimethyl sulfoxide (DMSO; Sigma-Aldrich, cat. no. D4540) in a 15-ml tube and store up to 6 weeks at 4° C. Do not repeat freeze-thawing cycles more than two times.

Fungizone, 250 μg/ml

Store undiluted Fungizone (Amphotericin B, Thermo Fisher Scientific, cat. no. 15290018) aliquots (1 ml) and store up to 12 months at −20° C. Thaw in water bath at 37° C. for 1 min prior to use.

### Hanks' Balanced Salt Solution Containing Dispase and Fungizone (HBSS-DF)

Dilute 50 U/ml Dispase (see recipe) in HBSS (Thermo Fisher Scientific, cat. no. 14175079) at 1:4 and add Fungizone (see recipe) to the final concentration of 0.5 μg/ml. To prepare a working solution for two tumor samples, add 400 μl of 50 U/ml Dispase and 4 μl of 250 μg/ml fungizone into 1.6 ml HBSS in 15-ml conical sterile polypropylene centrifuge tube. Prepare fresh.

### Hanks' Balanced Salt Solution Containing Dispase, Fungizone, Collagenase IV, and Y-27632 (HBSS-CFCY)

Add Collagenase IV and Y-27632 into HBSS-DF (see recipe) to the final concentration of 20 mg/ml and 10 μM, respectively. To prepare a working solution for two tumor samples, add 200 μl Collagenase IV (see recipe; 200 mg/ml) and 2 μl Y-27632 (see recipe; 10 mM) into 1.8 ml HBSS-DF into a 15-ml conical sterile polypropylene centrifuge tube (Thermo Fisher Scientific, cat. no. 14-959-53A). Prepare fresh.

### Hanks' Balanced Salt Solution Containing Dispase (HBSS-D)

Dilute 50 U/ml Dispase in HBSS at 1:4. To prepare a working solution that is sufficient to embed organoids grown in 3 wells, add 300 μl of 50 U/ml Dispase into 1200 μl HBSS. Prepare fresh.

### Matrigel® Matrix

Dispense Matrigel® matrix (Corning, cat. no. 354234) into 1.7-ml tubes (1 ml each) and store up to at −20° C. Thaw on ice for at least 1 hour or leave at 4° C. for 2-3 hours prior to use.

### Organoid Medium Containing Drugs

Make a stock solution by dissolving 1 mg of Paclitaxel (Selleck chemicals, cat. no. S1150) in 117 μl dimethyl sulfoxide (DMSO) to the final concentration of 10 mM and store at −20° C. Prepare tumor type-specific organoid medium containing Paclitaxel at a range of final concentrations (1.25, 2.5, 5.0, 10.0, 20.0 μM) by serial-dilution. Note that 100 μl is needed per well.

Make a stock solution by dissolving 65 mg 5-Fluorouracil (5-FU; (Sigma-Aldrich, cat. no. F6627)5-FU in 1 ml dimethyl sulfoxide (DMSO) to the final concentration of 65 mg/ml (500 mM) and store up to 3 months at −20° C. Prepare tumor type-specific organoid media containing 5-FU at a range of final concentrations (10⁻³, 10⁻², 10⁻¹, 1, 10, 100, 1000 μM) by serial-dilution. Note that 100 μl is needed per well.

Prepare fresh working solution by dissolving 0.5 mg of Cisplatin (Santa Cruz Biotechnology, cat. no. sc-200896) in 1 ml of 0.9% NaCl to the final concentration of 0.5 mg/ml (1.67 mM) Store up to 3 months at 4° C. Protect from light. Prepare tumor type-specific organoid medium containing Cisplatin at a range of final concentrations (1.0, 2.05, 4.1, 8.25, 16.5, 33.0 μM) by serial-dilution. Note that 100 μl is needed per well.

### Soybean Trypsin Inhibitor (STI)

Dissolve 250 mg STI (Sigma-Aldrich, cat. no. T9128) in 1000 ml Dulbecco's phosphate-buffered saline (DPBS; Thermo Fisher Scientific, cat. no. 14190250) (250 mg/L) and filter-sterile via a 1000-ml filter cup (e.g., Nalgene™ Rapid-Flow™ Sterile Disposable Filter Unit with PES Membrane; Thermo Fisher Scientific, cat. no. 167-0045), and dispense aliquots into 50-ml polypropylene tubes to be stored up to 6 months at 4° C.

### Y-27632, 10 mM

Dissolve 1 mg Y-27632 (ROCK1 inhibitor, Selleck Chemicals, cat. no. S1049) into 320 μl DPBS, dispense into 50-μl aliquots, and store at −20° C. Thaw at room temperature prior to use.

### Commentary

### Background Information

Esophageal cancer is the deadliest of all human cancers owing to late-stage presentation and diagnosis, therapy resistance, and/or early recurrence (Rustgi and El-Serag, 2014). Esophageal cancer comprises squamous cell carcinoma (ESCC) and adenocarcinoma (EAC), two major histologic subtypes. ESCC accounts for about 90% of esophageal cancers worldwide. The incidence of EAC has been rising in Western countries at an alarming rate. Intratumoral cancer cell heterogeneity contributes to therapy resistance in these cancers, and prediction of therapeutic outcomes in individual patients remains elusive.

The nomenclature of 3D organoids is somewhat controversial as they do not typically contain non-epithelial cellular components existing in the original tissues or organs [for historic review see (Moreira et al., 2018; Whelan et al., 2018)]. Among 3D cell culture systems, for example, organotypic 3D culture reconstitutes epithelia on top of the sub-epithelial stromal compartment comprising tissue-specific fibroblasts, type I collagen and basement membrane matrix (Matrigel) (Kalabis et al., 2012). Organotypic 3D culture has been utilized to model esophageal epithelial squamous-cell differentiation (Ohashi et al., 2010), ESCC development (Naganuma et al., 2012), and ESCC invasive tumor front (Grugan et al., 2010; Okawa et al., 2007). Organotypic 3D culture requires monolayer cultures of epithelial cells (normal or neoplastic) and fibroblasts prior to 3D reconstitution. By contrasts, 3D organoids are directly generated with cells isolated from starting tissue materials (e.g., endoscopic biopsies and surgically resected tumors).

Successful recapitulation of normal and malignant intestinal epithelial structures (i.e., intestinal organoids aka enteroids) has inspired similar attempts to grow and characterize epithelial structures from a variety of organs and tumor types using a defined set of growth factors, pharmacological agents and Matrigel (Sato et al., 2009; Sen et al., 2019). In recent years, PDO have been generated from multiple tumor types (Boj et al., 2015; Broutier et al., 2016; Driehuis et al., 2019; Gao et al., 2014; Huang et al., 2015; Hubert et al., 2016; Kijima et al., 2019; Lee et al., 2018; Li et al., 2018; Pauli et al., 2017; Sachs et al., 2018; Saito et al., 2019; Sato et al., 2011; Tanaka et al., 2018; Taneja, 2015; van de Wetering et al., 2015; Vlachogiannis et al., 2018; Walsh et al., 2016) as a more powerful tool in personalized/precision medicine, compared to other platforms such as patient-derived xenograft tumors. Indeed, PDO allow for rapid drug screening and prediction of therapy response in concert with automated imaging microscopies and other high-throughput multi-well screening tools. Furthermore, in conjunction with DNA- and RNA-sequencing approaches, PDO provide structural and functional insights into gastrointestinal oncology and more broadly into tumor biology.

We were the first to describe generation of PDO from oral/head-and-neck and esophageal squamous cell carcinomas (SCCs; OSCC, HNSCC and ESCC) to evaluate therapy response and interrogate therapy resistance mechanisms (Kijima et al., 2019). We have further optimized organoid culture conditions to support the growth of PDO from broader SCCs (OSCC, HNSCC, anal SCC) as well as EAC according to the above-described protocols. These methods may be applied to cancers of other organs, such as the uterus and the lung where both squamous cell carcinomas and adenocarcinomas develop (Lin et al., 2017).

### Critical Parameters, Understanding Results, and Troubleshooting

Key parameters in successful PDO generation and characterization (FIG. 36) include quality of starting materials, enzymatic digestion and mechanical dissociation and straining (i.e., cell viability and cellular stress during transportation and tissue dissociation), as well as control of bacterial and fungal contamination. Medium components and Matrigel are essential reagents. Daily observation, monitoring of growth kinetics, as well as appropriate training in cell culture and other relevant techniques (e.g., histology, flow cytometry) will ensure successful generation of organoid lines. Employment of quality control reporter cell lines, standard molecular markers, functional and morphological analyses of 3D organoid products are key for ensuring that generated organoid lines are viable and recapitulate the original tumor specimen.

### Starting Materials

Freshly isolated biopsies and surgical specimens serve best for successful PDO generation. After procurement, these clinical materials should be protected against ischemic conditions and thus processed as soon as possible to sustain cell viability. Surgically resected tumors often contain necrotic tissue that should be removed. A higher cell viability in the starting materials is helpful to gain an increased yield in the primary 3D organoid products, permitting more diverse subsequent phenotypic analyses (e.g., genetic profiling, morphology, flow cytometry, and drug response), cryopreservation and subculture. Due to the obstructive nature of esophageal tumors (esophageal stricture, dysphagia, food impactions), primary cultures of esophageal cancer specimens are at high risk of fungal and/or bacterial contamination. Addition of fungicides and antibiotics to all solutions and extensive rinsing of the tissue with large volumes of DPBS reduces this risk. Tissue samples should be placed in chilled DPBS or an organ transplant preserving solution (University of Wisconsin solution aka Belzer UW® Cold Storage Solution, Bridge to Life, cat. no. BUW-005), the latter for overnight shipping of tissues from a collection site. In our hands, freezing of specimens has diminished organoid formation rate.

### Sample Processing

Enzymatic and mechanical dissociation of the tissue and the use of cell strainer are necessary; however, such processes can cause isolation-related stress. In a single-cell suspension, ROCK inhibitor Y-27632 is utilized to minimize cell death associated with loss of cell-matrix or cell-cell contact (anoikis). One may argue that organoid formation rate may be improved by starting with cell aggregates instead of a well-dissociated single-cell suspension; however, this does not appear to be the case in our extensive experience.

### Medium Components, Growth Conditions, and Quality Control Cell Lines

Certain components of the organoid culture media, particularly the recombinant proteins in the L-WRN- or RN-conditioned media, have a short half-life. It is recommended storing complete organoid growth media for no longer than 2 weeks. Growth factors and agents included in organoid media have been optimized; however, it remains elusive whether each of them is necessary, sufficient, or even beneficial for all esophageal cancer organoids from different patients. ESCC PDO tend to grow more successfully from patients with poorly-differentiated ESCC, and those showing therapy resistance (Kijima et al., 2019). It is possible that current organoid media may be selective for a subset of cancer cells (e.g., cancer stem cells characterized by high CD44 expression) within individual tumors.

Advanced DMEM/F12 is often used as a base medium to grow 3D organoids; however, media and their constitutes vary from study to study. To generate 3D organoids from human primary SCCs of oral (OSCC)/head-and-neck (HNSCC), esophagus (ESCC) and anal origin, in the present disclosure Wnt3A, A83-01, Nicotinamide and SB202190 were omitted from the medium originally described (Kijima et al., 2019). Removal of these factors did not affect formation and growth of primary or passaged 3D organoids in our ongoing practice (H. Maekawa & M. Shimonosono, unpub. observ). This medium composition works well for murine ESCC 3D organoids (Natsuizaka et al., 2017) except that speciesspecific recombinant EGF (i.e., human EGF for human organoids) is utilized. Although patient-derived ESCC 3D organoids have not been described elsewhere, 3D organoids have been generated from OSCC/HNSCC patients (Driehuis et al., 2019; Driehuis et al., 2019; Tanaka et al., 2018). Driehuis et al. utilize advanced DMEM/F12 where they supplement CHIR99021 (GSK3β inhibitor), FGF2, FGF10, Prostaglandin E, and forskolin that we do not use. While their success rate (˜60%) to form HNSCC 3D organoid is comparable with ours (˜70%), their medium did not significantly improve our PDO growth efficiency (H. Maekawa & M. Shimonosono, unpub. observ.). Tanaka et al. utilized a human embryonic stem cell culture medium supplemented with bFGF; however, their success rate (30%) was lower than ours. Li et al. are the first to describe EAC 3D organoids from multiple patients (Li et al., 2018). Utilizing advanced DMEM/F12, their medium does not contain CHIR99021, Gastrin, Y-27632 and the N-2 supplement we use based on the principle set by Sato et al. to grow 3D organoids of intestinal cell types (Sato et al., 2011). The success rate by Li et al. is reported 31% while ours is ˜80%. Of note, their condition did not support 3D organoids from Barrett's esophagus (i.e., intestinal metaplasia), a histologic precursor of EAC.

Among commonly utilized growth supplements, it is suggested that EGF, NAC, N2, and B27 are necessary although both N2 and B27 supplements include unknown concentrations of factors (insulin, transferrin, and selenite) that are redundantly present in the advanced DMEM/F12 base medium as well. A83-01 would be beneficial in most cases as we find useful to grow EAC, but not ESCC, 3D organoids. Given lot-to-lot variability in Matrigel, growth factors, antioxidants and other agents in media, we have utilized extensively characterized esophageal cancer cell lines (e.g., TE11, OE-33) (Kijima et al., 2019) to evaluate organoid formation in each medium tested for quality control purposes. Additionally, newly prepared L-WRN and RN can be tested in standard enteroid culture conditions. We use cell culture incubators set at 5% CO₂, 37° C., >95% humidity to grow PDO. Growth conditions such as low oxygen tension (i.e., hypoxia) (Fujii et al., 2016) and air-liquid interface (Li et al., 2016) remain yet to be validated for esophageal cancer PDO.

Growing cancer cells in primary culture meets often a common technical issue of concurrent growth of non-cancer cells. In Matrigel droplet, non-epithelial cells (e.g., fibroblasts and immune cells) do not form 3D structures; however, normal epithelial or precancerous cells may form 3D organoids along with ESCC (and OSCC/HNSCC) cells (Kijima et al., 2019). In our EAC 3D organoid culture conditions, normal esophageal (squamous epithelial cell) organoids do not grow; however, 3D structures compatible with Barrett's esophagus (i.e., intestinal metaplasia) may grow concurrently (T. A. Karakasheva, J. T. Gabre, and R. Cruz-Acuña, unpub. observ.). The size and morphology (phase contrast images and H&E staining) of organoids will help us to distinguish non-cancerous 3D structures from cancerous structures. Additionally, normal organoids do not grow continuously beyond 14 days in culture. Drug sensitivity to chemotherapeutic agents may be overestimated when the proportion of non-cancerous 3D organoids is significant in primary 3D organoid culture. It is important to follow 3D organoid structures that remain viable following exposure to chemotherapy agents. If necessary, drugs should be tested on passaged secondary 3D organoids where cancerous organoids become predominant over non-cancerous organoids (Kijima et al., 2019). Of note, non-neoplastic human esophageal 3D organoids grow better and display a more exquisite proliferation-differentiation gradient in media with distinct compositions (Kasagi et al., 2018) with detailed protocols provided in the complementary manuscript (Nakagawa et al., Current Protocols Stem Cell Biology, In press). Outgrowth of stromal fibroblasts is a cumbersome issue in primary monolayer culture. Growth of fibroblasts were occasionally observed on the plastic surface in the organoid culture plates; however, fibroblasts are not transferred into subsequent passages because they remain adhered on the plastic surface when we harvest organoids in Matrigel without utilizing trypsin (Basic Protocol 2 steps 2-3).

### Cell Numbers to be Seeded and Growth Kinetics

Given a relatively low organoid formation rate (0.01%-1%) anticipated from human tissue materials, 20,000 viable cells are typically seeded per well in 24-well plates to initiate organoid cultures yielding 20-200 PDO per well. When passaged, organoid formation rate is anticipated to improve by 10-fold, permitting seeding of a smaller number of cells (e.g., 2000 per well in 24-well plates; and 200 per well in 96-well plates). It should be noted that these live cells are usually seeded along with co-existing dead cells. Dead cells exude enzymes that degrade extracellular matrix components in Matrigel. Thus, seeding more cells does not necessarily increase the organoid formation rate. Trypan blue exclusion test detects dead cells, but not necessarily dying cells, overestimating the number of live cells seeded. Thus, high cell viability in the original tumor is crucial for a successful primary PDO culture, reinforcing the appropriate preservation, transportation and processing of the starting materials.

Live cancer cells also secrete matrix degrading enzymes such as matrix metalloproteases. As the organoids grow, the Matrigel may become loose and break apart. To preserve the Matrigel structure, media should be added gently and only along the wall of the well. If the Matrigel breaks, the organoids may be pelleted without trypsinization and re-embedded in fresh Matrigel. When PDO are initiated successfully, spherical structures emerge within 4-7 days and continue to grow to be passaged by day 11-14. We do not determine routinely doubling time of cancer cells within 3D organoids for a practical reason to save as many organoids-containing wells for morphological characterization and drug-testing. If necessary, we can determine the average number of live cells present in each 3D structure. By harvesting exponentially growing organoids, we have estimated doubling time of esophageal cancer PDO at approximately 24-36 hours with each organoid starting from a single cell in suspension. This estimate ignores the initial lag phase, thereby raising the possibility of underestimation. The mature PDO may reach 100-250 μm in diameter. Overgrown PDO tend to contain an internal necrotic core as detected by H&E staining and should be avoided. The high number of metabolically active cells lowers the media pH which can be monitored by medium color turning to yellow a day after medium change.

### Contaminations

Clinical esophageal tumor samples are vulnerable to bacterial and fungal (yeast and mold) contaminations, as noted in the above “starting materials” section. It is recommended to have tissue culture incubators devoted for the use of primary organoid cultures. Fungicides and antibiotics may fail to prevent contamination. Bacteria and yeast grow rapidly, typically overnight. The suspected culture should be quarantined and may be treated with higher concentration (50 μg/ml) of gentamycin. Fungus-contaminated plates should be discarded immediately. Fungal contamination remains the biggest hindering factor in generation of organoids from esophageal cancer.

### Molecular and Functional Characteristics of Esophageal Cancer PDO

Analyses of PDO by standard assays such as histology (hematoxylin and eosin staining), immunohistochemistry and flow cytometry reveal unique attributes of esophageal cancer cells constituting individual organoid structures grown in each well (FIGS. 37A-37B). Histology can define the degree of atypia and differentiation grade of cancer cells. While normal organoids display a well-organized differentiation gradient reminiscent of the stratified squamous-cell differentiation in the normal esophageal epithelium (Kasagi et al., 2018), cancerous organoids display a various degree of atypia and abnormal differentiation with increased proliferation, which can be documented by immunohistochemistry and immunofluorescence for markers such as Ki-67 (Kijima et al., 2019). ESCC cells may display upregulation of SOX2 (Dotto and Rustgi, 2016; Watanabe et al., 2014), while EAC cells may express CDX2 (Lord et al., 2005). Upregulation of dysfunctional tumor suppressor TP53 protein is common in both ESCC and EAC via mutant TP53 stabilization (Dotto and Rustgi, 2016; Fisher et al., 2017); however, some tumors display TP53 downregulation via other mechanisms (e.g., epigenetic silencing). It should be noted that such molecular changes in PDO recapitulate those in the original tumors (FIGS. 37A-37B). Additionally, these marker expression patterns in PDO are stable between passages. Flow cytometry may reveal a subset of cancer cells with elevated expression of CD44, which may have properties of tumor initiating cells or cancer stem (stemlike) cells (Natsuizaka et al., 2017; Whelan et al., 2017). When isolated by fluorescence-activated cell sorting (FACS), such cells can be further propagated (Basic Protocol 2) to assess their organoid formation capability and may show an increased organoid formation capability when passaged in subsequent organoid culture. Flow cytometry may also detect unique cellular functions and processes such as autophagy (Kijima et al., 2019; Whelan et al., 2017) and epithelial-tomesenchymal transition (EMT) (Karakasheva et al., 2018; Kinugasa et al., 2015; Natsuizaka et al., 2017), which may contribute to therapy resistance mechanisms. Additionally, PDO may be lysed for conventional gene expression analyses (e.g., immunoblotting, quantitative reverse-transcription polymerase chain reaction, RNA-sequencing) or single cell-RNA sequencing, and genetic and epigenetic profiling (e.g., whole exome sequencing). Such approach has been increasingly crucial for not only molecular subtyping of tumors but validation of the fidelity of PDO through a head-to-head comparison of molecular and functional phenotypes (e.g., specific tissue markers and drug response) and genomic alterations between original tumors and the resulting PDO.

### Success Rates

Generation of primary organoids within the 14 days qualifies as success. Success rate of PDO generation is 60% (n=25) for ESCC and 80% (n=6) for EAC. Primary EAC PDO are readily passaged times. ESCC PDO are harder to passage than EAC PDO as ˜10% of primary ESCC PDO can be passaged times. This presents the foremost limitation for PDO generated from squamous cell carcinomas. Interestingly, our PDO culture conditions are permissive for 3D organoid generation from HNSCC and ESCC cell lines (100%, n=5) and HNSCC/ESCC patient derived xenograft tumors (PDX) (80%, n=5). The 3D organoids from these established cell lines and PDX are easily passaged in our PDO culture medium (H. Maekawa & M. Shimonosono, unpub. observ.). Genetic profiling of these cell line/PDX-derived 3D organoids is currently underway, comparing to primary PDO, with a hope to identify signaling pathways or genetic factors that PDO to be passaged more successfully.

### Time Considerations

It is recommended that the tissue sample be processed as soon as possible after procurement from the patient. Single-cell suspension is typically generated in 1-2 hours, followed by solidification of Matrigel domes for 30 min. The organoids are ready for passage and/or harvest in up to 14 days (roughly 10 days for ESCC and 14 days for EAC). Preparation of cultures and drug treatment for IC50 determination takes 8-11 days.

### REFERENCES

Al-Hajj M, Wicha M S, Benito-Hernandez A, Morrison S J, Clarke M F. Prospective identification of tumorigenic breast cancer cells. Proc Natl Acad Sci USA. 2003; 100(7):3983-8. Epub 2003/03/12. doi: 10.1073/pnas.05302911000530291100 [pii]. PubMed PMID: 12629218; PMCID: 153034.
Basu D, Nguyen T T K, Montone K T, Zhang G, Wang L P, Diehl J A, Rustgi A K, Lee J T, Weinstein G S, Herlyn M. Evidence for mesenchymal-like sub-populations within squamous cell carcinomas possessing chemoresistance and phenotypic plasticity. Oncogene. 2010; 29(29):4170-82. doi: Doi 10.1038/Onc.2010.170. PubMed PMID: ISI:000280151500005.
Blanchard, C., Mingler, M. K., Vicario, M., Abonia, J. P., Wu, Y. Y., Lu, T. X., Collins, M. H., Putnam, P. E., Wells, S. I., and Rothenberg, M. E. 2007. IL-13 involvement in eosinophilic esophagitis: transcriptome analysis and reversibility with glucocorticoids. The Journal of allergy and clinical immunology 120:1292-1300.
Boj, S. F., Hwang, C. I., Baker, L. A., Chio, I. I., Engle, D. D., Corbo, V., . . . Tuveson, D. A. (2015). Organoid models of human and mouse ductal pancreatic cancer. Cell, 160(1-2), 324-338.
Broutier, L., Andersson-Rolf, A., Hindley, C. J., Boj, S. F., Clevers, H., Koo, B. K., & Huch, M. (2016). Culture and establishment of self-renewing human and mouse adult liver and pancreas 3D organoids and their genetic manipulation. Nature Protocols, 11(9), 1724-1743.
Christofori G. New signals from the invasive front. Nature. 2006; 441(7092):444-50. Epub 2006/05/26. doi: nature04872 [pii] 10.1038/nature04872. PubMed PMID: 16724056.
DeWard, A. D., Cramer, J., and Lagasse, E. 2014. Cellular heterogeneity in the mouse esophagus implicates the presence of a nonquiescent epithelial stem cell population. Cell reports 9:701-711.
Dotto, G. P., & Rustgi, A. K. (2016). Squamous cell cancers: A unified perspective on biology and genetics. Cancer Cell, 29(5), 622-637.
Driehuis, E., Kolders, S., Spelier, S., Lohmussaar, K., Willems, S. M., Devriese, L. A., . . . Clevers, H. (2019). Oral mucosal organoids as a potential platform for personalized cancer therapy. Cancer Discovery, 9(7), 852-871.
Driehuis, E., Spelier, S., Beltran Hernandez, I., de Bree, R., Willems, S. M., Clevers, H., & Oliveira, S. (2019). Patient-derived head and neck cancer organoids recapitulate EGFR expression levels of respective tissues and are responsive to EGFR-targeted photodynamic therapy. Journal of Clinical Medicine, 8(11), pii: E1880.
Fisher, O. M., Lord, S. J., Falkenback, D., Clemons, N. J., Eslick, G. D., & Lord, R. V. (2017). The prognostic value of TP53 mutations in oesophageal adenocarcinoma: A systematic review and meta-analysis. Gut, 66(3), 399-410.
Fujii, M., Shimokawa, M., Date, S., Takano, A., Matano, M., Nanki, K., . . . Sato, T. (2016). A colorectal tumor organoid library demonstrates progressive loss of niche factor requirements during tumorigenesis. Cell Stem Cell, 18(6), 827-838.
Gal A A, Martin S E, Kernen J A, Patterson M J. Esophageal carcinoma with prominent spindle cells. Cancer. 1987; 60(9):2244-50. Epub 1987/11/01. PubMed PMID: 2449943.
Gao, D., Vela, I., Sboner, A., laquinta, P. J., Karthaus, W. R., Gopalan, A., . . . Chen, Y. (2014). Organoid cultures derived from patients with advanced prostate cancer. Cell, 159(1), 176-187.
Giroux V, Lento A A, Islam M, Pitarresi J R, Kharbanda A, Hamilton K E, Whelan
K A, Long A, Rhoades B, Tang Q, Nakagawa H, Lengner C J, Bass A J, Wileyto E P, Klein-Szanto A J, Wang T C, Rustgi A K. Long-lived keratin 15+ esophageal progenitor cells contribute to homeostasis and regeneration. J Clin Invest. 2017; 127(6):2378-91. Epub 2017/05/10. doi: 10.1172/JC188941. PubMed PMID: 28481227; PMCID: PMC5451220.
Grugan, K. D., Miller, C. G., Yao, Y., Michaylira, C. Z., Ohashi, S., Klein-Szanto, A. J., . . . Rustgi, A. K. (2010). Fibroblast-secreted hepatocyte growth factor plays a functional role in esophageal squamous cell carcinoma invasion. Proceedings of the National Academy of Sciences of the United States of America, 107(24), 11026-11031.
Gustafsson M V, Zheng X, Pereira T, Gradin K, Jin S, Lundkvist J, Ruas J L, Poellinger L, Lendahl U, Bondesson M. Hypoxia requires notch signaling to maintain the undifferentiated cell state. Dev Cell. 2005; 9(5):617-28. Epub 2005/11/01. doi: S1534-5807(05)00372-2 [pii] 10.1016/j.devcel.2005.09.010. PubMed PMID: 16256737.
Harada, H., Nakagawa, H., Oyama, K., Takaoka, M., Andl, C. D., Jacobmeier, B., Werder, von, A., Enders, G. H., Opitz, O. G., and Rustgi, A. K. 2003. Telomerase induces immortalization of human esophageal keratinocytes without p16INK4a inactivation. Molecular cancer research: MCR 1:729-738.
Huang, L., Holtzinger, A., Jagan, I., BeGora, M., Lohse, I., Ngai, N., . . . Muthuswamy, S. K. (2015). Ductal pancreatic cancer modeling and drug screening using human pluripotent stem cell- and patient-derived tumor organoids. Nature Medicine, 21(11), 1364-1371.
Hubert, C. G., Rivera, M., Spangler, L. C., Wu, Q., Mack, S. C., Prager, B. C., . . . Rich, J. N. (2016). A three-dimensional organoid culture system derived from human glioblastomas recapitulates the hypoxic gradients and cancer stem cell heterogeneity of tumors found in vivo. Cancer Research, 76(8), 2465-2477.
Kalabis, J., Wong, G. S., Vega, M. E., Natsuizaka, M., Robertson, E. S., Herlyn, M., . . . Rustgi, A. K. (2012). Isolation and characterization of mouse and human esophageal epithelial cells in 3D organotypic culture. Nature Protocols, 7(2), 235-246.
Karakasheva T A, Kijima T, Shimonosono M, Maekawa H, Sahu V, Gabre J T, Cruz-Acuna R, Giroux V, Sangwan V, Whelan K A, Natsugoe S, Yoon A J, Philipone E, Klein-Szanto A J, Ginsberg G G, Falk G W, Abrams J A, Que J, Basu D, Ferri L, Diehl J A, Bass A J, Wang T C, Rustgi A K, Nakagawa H. Generation and Characterization of Patient-Derived Head and Neck, Oral, and Esophageal Cancer Organoids. Curr Protoc Stem Cell Biol. 2020; 53(1):e109. Epub 2020/04/16. doi: 10.1002/cpsc.109. PubMed PMID: 32294323; PMCID: PMC7350550.
Karakasheva, T. A., Lin, E. W., Tang, Q., Qiao, E., Waldron, T. J., Soni, M., . . . Rustgi, A. K. (2018). IL-6 mediates cross-talk between tumor cells and activated fibroblasts in the tumor microenvironment. Cancer Research, 78(17), 4957-4970.
Kasagi, Y., Chandramouleeswaran, P. M., Whelan, K. A., Tanaka, K., Giroux, V., Sharma, M., . . . Nakagawa, H. (2018). The esophageal organoid system reveals functional interplay between notch and cytokines in reactive epithelial changes. Cellular and Molecular Gastroenterology and Hepatology, 5(3), 333-352
Kasagi, Y., Dods, K., Wang, J. X., Chandramouleeswaran, P. M., Benitez, A. J., Gambanga, F., Kluger, J., Ashorobi, T., Gross, J., Tobias, J. W., et al. 2018b. Fibrostenotic eosinophilic esophagitis may reflect epithelial lysyl oxidase induction by fibroblasts-derived tumor necrosis factor-α. The Journal of allergy and clinical immunology.
Kijima, T., Nakagawa, H., Shimonosono, M., Chandramouleeswaran, P. M., Hara, T., Sahu, V., . . . Natsugoe, S. (2019). Three-dimensional organoids reveal therapy resistance of esophageal and oropharyngeal squamous cell carcinoma cells. Cellular and Molecular Gastroenterology and Hepatology, 7(1), 73-91.
Kinugasa, H., Whelan, K. A., Tanaka, K., Natsuizaka, M., Long, A., Guo, A., . . . Nakagawa, H. (2015). Mitochondrial SOD2 regulates epithelial-mesenchymal transition and cell populations defined by differential CD44 expression. Oncogene, 34(41), 5229-5239.
Lee, S. H., Hu, W., Matulay, J. T., Silva, M. V., Owczarek, T. B., Kim, K., . . . Shen, M. M. (2018). Tumor evolution and drug response in patient-derived organoid models of bladder cancer. Cell, 173(2), 515-528 e517.
Li, X., Francies, H. E., Secrier, M., Perner, J., Miremadi, A., Galeano-Dalmau, N., . . . Garnett, M. J. (2018). Organoid cultures recapitulate esophageal adenocarcinoma heterogeneity providing a model for clonality studies and precision therapeutics. Nature Communications, 9(1), 2983.
Li, X., Ootani, A., & Kuo, C. (2016). An air-liquid interface culture system for 3D organoid culture of diverse primary gastrointestinal tissues. Methods in Molecular Biology, 1422, 33-40.
Lim, D. M., Narasimhan, S., Michaylira, C. Z., and Wang, M.-L. 2009. TLR3-mediated NF-{kappa}B signaling in human esophageal epithelial cells. American journal of physiology. Gastrointestinal and liver physiology 297:G1172-80.
Lin, E. W., Karakasheva, T. A., Lee, D. J., Lee, J. S., Long, Q., Bass, A. J., . . . Rustgi, A. K. (2017). Comparative transcriptomes of adenocarcinomas and squamous cell carcinomas reveal molecular similarities that span classical anatomic boundaries. Plos Genetics, 13 (8), e1006938.
Liotta L A, Kohn E C. The microenvironment of the tumour-host interface. Nature. 2001; 411(6835):375-9. Epub 2001/05/18. doi: 10.1038/3507724135077241 [pii]. PubMed PMID: 11357145.
Long A, Giroux V, Whelan K A, Hamilton K E, Tetreault M P, Tanaka K, Lee J S, Klein-Szanto A J, Nakagawa H, Rustgi A K. WNT10A promotes an invasive and self-renewing phenotype in esophageal squamous cell carcinoma. Carcinogenesis. 2015; 36(5):598-606. Epub 2015/03/22. doi: 10.1093/carcin/bgv025. PubMed PMID: 25795715; PMCID: PMC4498148.
Lord, R. V., Brabender, J., Wickramasinghe, K., DeMeester, S. R., Holscher, A., Schneider, P. M., . . . DeMeester, T. R. (2005). Increased CDX2 and decreased PITX1 homeobox gene expression in Barrett's esophagus and Barrett's-associated adenocarcinoma. Surgery, 138(5), 924-931.
Markopoulos A K. Current aspects on oral squamous cell carcinoma. Open Dent J. 2012; 6:126-30. Epub 2012/08/30. doi: 10.2174/1874210601206010126. PubMed PMID: 22930665; PMCID: PMC3428647.
Miyoshi, H., & Stappenbeck, T. S. (2013). In vitro expansion and genetic modification of gastrointestinal stem cells in spheroid culture. Nature Protocols, 8(12), 2471-2482.
Moreira, L., Bakir, B., Chatterji, P., Dantes, Z., Reichert, M., & Rustgi, A. K. (2018). Pancreas 3D organoids: Current and future aspects as a research platform for personalized medicine in pancreatic cancer. Cellular and Molecular Gastroenterology and Hepatology, 5(3), 289-298.
Muir, A. B., Lim, D. M., Benitez, A. J., Modayur Chandramouleeswaran, P., Lee, A. J., Ruchelli, E. D., Spergel, J. M., and Wang, M.-L. 2013. Esophageal epithelial and mesenchymal cross-talk leads to features of epithelial to mesenchymal transition in vitro. Experimental cell research 319:850-859.
Naganuma, S., Whelan, K. A., Natsuizaka, M., Kagawa, S., Kinugasa, H., Chang, S., . . . Nakagawa, H. (2012). Notch receptor inhibition reveals the importance of cyclin D1 and Wnt signaling in invasive esophageal squamous cell carcinoma. American Journal of Cancer Research, 2(4), 459-475.
Nakagawa H, Kasagi Y, Karakasheva T A, Hara T, Aaron B, Shimonosono M, Kijima T, Giroux V, Bailey D, Wilkins B, Abrams J A, Falk G W, Aceves S S, Spergel J M, Hamilton K E, Whelan K A, Muir A B. Modeling Epithelial Homeostasis and Reactive Epithelial Changes in Human and Murine Three-Dimensional Esophageal Organoids. Curr Protoc Stem Cell Biol. 2020; 52(1):e106. Epub 2020/02/28. doi: 10.1002/cpsc.106. PubMed PMID: 32105412; PMCID: PMC7288850.
Natsuizaka, M., Whelan, K. A., Kagawa, S., Tanaka, K., Giroux, V., Chandramouleeswaran, P. M., . . . Nakagawa, H. (2017). Interplay between Notch1 and Notch3 promotes EMT and tumor initiation in squamous cell carcinoma. Nature Communications, 8(1), 1758.
Neville B W; Day T A. Oral cancer and precancerous lesions. C A Cancer J Clin. 2002; 52(4):195-215. Epub 2002/07/26. doi: 10.3322/canjclin.52.4.195. PubMed PMID: 12139232.
Nguyen, N., Fernando, S. D., Biette, K. A., Hammer, J. A., Capocelli, K. E., Kitzenberg, D. A., Glover, L. E., Colgan, S. P., Furuta, G. T., and Masterson, J. C. 2017. TGF-β1 alters esophageal epithelial barrier function by attenuation of claudin-7 in eosinophilic esophagitis. Mucosal immunology 1:729.
Ohashi, S., Natsuizaka, M., Yashiro-Ohtani, Y., Kalman, R. A., Nakagawa, M., Wu, L., . . . Nakagawa, H. (2010). NOTCH1 and NOTCH3 coordinate esophageal squamous differentiation through a CSL-dependent transcriptional network. Gastroenterology, 139(6), 2113-2123.
Okawa, T., Michaylira, C. Z., Kalabis, J., Stairs, D. B., Nakagawa, H., Andl, C. D., . . . Rustgi, A. K. (2007). The functional interplay between EGFR overexpression, hTERT activation, and p53 mutation in esophageal epithelial cells with activation of stromal fibroblasts induces tumor development, invasion, and differentiation. Genes & Development, 21(21), 2788-2803.
Pauli, C., Hopkins, B. D., Prandi, D., Shaw, R., Fedrizzi, T., Sboner, A., . . . Rubin, M. A. (2017). Personalized in vitro and in vivo cancer models to guide precision medicine. Cancer Discovery, 7(5), 462-477.
Pouw R E, Gondrie J J, Rygiel A M, Sondermeijer C M, ten Kate F J, Odze R D, Vieth M, Krishnadath K K, Bergman J J. Properties of the neosquamous epithelium after radiofrequency ablation of Barrett's esophagus containing neoplasia. Am J Gastroenterol. 2009; 104(6):1366-73. doi: 10.1038/ajg.2009.88. PubMed PMID: 19491850.
Prince M E, Sivanandan R, Kaczorowski A, Wolf G T, Kaplan M J, Dalerba P, Weissman I L, Clarke M F, Ailles L E. Identification of a subpopulation of cells with cancer stem cell properties in head and neck squamous cell carcinoma. Proc Natl Acad Sci USA. 2007; 104(3):973-8. Epub 2007/01/11. doi: 0610117104 [pii] 10.1073/pnas.0610117104. PubMed PMID: 17210912; PMCID: 1783424.
Rosekrans, S. L., Baan, B., Muncan, V., and van den Brink, G. R. 2015. Esophageal development and epithelial homeostasis. American journal of physiology. Gastrointestinal and liver physiology 309:G216-28.
Ruffner, M. A., Song, L., Maurer, K., Shi, L., Carroll, M. C., Wang, J. X., Muir, A. B., Spergel, J. M., and Sullivan, K. E. 2019. Toll-like receptor 2 stimulation augments esophageal barrier integrity. Allergy 1:729.
Rustgi, A. K., & E I-Serag, H. B. (2014). Esophageal carcinoma. New England Journal of Medicine, 371(26), 2499-2509.
Sachs, N., de Ligt, J., Kopper, O., Gogola, E., Bounova, G., Weeber, F., . . . Clevers, H. (2018). A living biobank of breast cancer organoids captures disease heterogeneity. Cell, 172(1-2), 373-386 e310.
Saito, Y., Muramatsu, T., Kanai, Y., Ojima, H., Sukeda, A., Hiraoka, N., . . . Saito, H. (2019). Establishment of patient-derived organoids and drug screening for biliary tract carcinoma. Cell Reports, 27(4), 1265-1276 e1264.
Salaspuro V, Salaspuro M. Synergistic effect of alcohol drinking and smoking on in vivo acetaldehyde concentration in saliva. Int J Cancer. 2004; 111(4):480-3. Epub 2004/07/09. doi: 10.1002/ijc.20293. PubMed PMID: 15239123.
Sato, T., Stange, D. E., Ferrante, M., Vries, R. G., van Es, J. H., van den Brink, S., . . . Clevers, H. (2011). Long-term expansion of epithelial organoids from human colon, adenoma, adenocarcinoma, and Barrett's epithelium. Gastroenterology, 141(5), 1762-1772.
Sato, T., Vries, R. G., Snippert, H. J., van de Wetering, M., Barker, N., Stange, D. E., . . . Clevers, H. (2009). Single Lgr5 stem cells build cryptvillus structures in vitro without a mesenchymal niche. Nature, 459(7244), 262-265.
Sen, M., Hahn, F., Black, T. A., DeMarshall, M., Porter, W., Snowden, E., . . . Carpenter, E. L. (2019). Correction: Flow based single cell analysis of the immune landscape distinguishes Barrett's esophagus from adjacent normal tissue. Oncotarget, 10(49), 5119.
Sherrill, J. D., Kiran, K. C., Blanchard, C., Stucke, E. M., Kemme, K. A., Collins, M. H., Abonia, J. P., Putnam, P. E., Mukkada, V. A., Kaul, A., et al. 2014. Analysis and expansion of the eosinophilic esophagitis transcriptome by RNA-sequencing. Genes and immunity 15:361-369.
Stairs D B, Bayne L J, Rhoades B, Vega M E, Waldron T J, Kalabis J, Klein-Szanto A, Lee J S, Katz J P, Diehl J A, Reynolds A B, Vonderheide R H, Rustgi A K. Deletion of p120-catenin results in a tumor microenvironment with inflammation and cancer that establishes it as a tumor suppressor gene. Cancer Cell. 2011; 19(4):470-83. doi: 10.1016/j.ccr.2011.02.007. PubMed PMID: 21481789; PMCID: PMC3077713.
Takayama N, Arima S, Haraoka S, Kotho T, Futami K, Iwashita A. Relationship between the expression of adhesion molecules in primary esophageal squamous cell carcinoma and metastatic lymph nodes. Anticancer Res. 2003; 23(6a):4435-42. Epub 2003/12/12. PubMed PMID: 14666731.
Tanaka K, Whelan K A, Chandramouleeswaran P M, Kagawa S, Rustgi S L, Noguchi C, Guha M, Srinivasan S, Amanuma Y, Ohashi S, Muto M, Klein-Szanto A J, Noguchi E, Avadhani N G, Nakagawa H. ALDH2 modulates autophagy flux to regulate acetaldehyde-mediated toxicity thresholds. Am J Cancer Res. 2016; 6(4):781-96. Epub 2016/05/18. PubMed PMID: 27186430; PMCID: PMC4859883.
Tanaka, N., Osman, A. A., Takahashi, Y., Lindemann, A., Patel, A. A., Zhao, M., . . . Myers, J. N. (2018). Head and neck cancer organoids established by modification of the CTOS method can be used to predict in vivo drug sensitivity. Oral Oncology, 87, 49-57.
Taneja, S. S. (2015). Re: Organoid cultures derived from patients with advanced prostate cancer. Journal of Urology, 193(4), 1442-1443.
Tang X H, Knudsen B, Bemis D, Tickoo S, Gudas L J. Oral cavity and esophageal carcinogenesis modeled in carcinogen-treated mice. Clin Cancer Res. 2004; 10(1 Pt 1):301-13. Epub 2004/01/22. PubMed PMID: 14734483.
Toole B P, Slomiany M G. Hyaluronan, CD44 and Emmprin: partners in cancer cell chemoresistance. Drug Resist Updat. 2008; 11(3):110-21. Epub 2008/05/21. doi: S1368-7646(08)00021-6 [pii] 10.1016/j.drup.2008.04.002. PubMed PMID: 18490190; PMCID: 2584579.
Twarock S, Tammi M I, Savani R C, Fischer J W. Hyaluronan stabilizes focal adhesions, filopodia, and the proliferative phenotype in esophageal squamous carcinoma cells. J Biol Chem. 2010; 285(30):23276-84. Epub 2010/05/14. doi: M109.093146 [pii] 10.1074/jbc.M109.093146. PubMed PMID: 20463012; PMCID: 2906320.
Uchikado Y, Natsugoe S, Okumura H, Setoyama T, Matsumoto M, Ishigami S, Aikou T. Slug Expression in the E-cadherin preserved tumors is related to prognosis in patients with esophageal squamous cell carcinoma. Clin Cancer Res. 2005; 11(3):1174-80. PubMed PMID: 15709186.
Usami Y, Satake S, Nakayama F, Matsumoto M, Ohnuma K, Komori T, Semba S, Ito A, Yokozaki H. Snail-associated epithelial-mesenchymal transition promotes oesophageal squamous cell carcinoma motility and progression. J Pathol. 2008; 215(3):330-9. PubMed PMID: 18491351. van de Wetering, M., Francies, H. E., Francis, J. M., Bounova, G., Iorio, F., Pronk,
A., . . . Clevers, H. (2015). Prospective derivation of a living organoid biobank of colorectal cancer patients. Cell, 16/(4), 933-945.
Vermorken J B, Mesia R, Rivera F, Remenar E, Kawecki A, Rottey S, Erfan J, Zabolotnyy D, Kienzer H R, Cupissol D, Peyrade F, Benasso M, Vynnychenko I, De Raucourt D, Bokemeyer C, Schueler A, Amellal N, Hitt R. Platinum-based chemotherapy plus cetuximab in head and neck cancer. N Engl J Med. 2008:359(11):1116-27. Epub 2008/09/12. doi: 10.1056/NEJMoa0802656. PubMed PMID: 18784101.
Vlachogiannis, G., Hedayat, S., Vatsiou, A., Jamin, Y., Fernandez-Mateos, J., Khan, K., . . . Valeri, N. (2018). Patient-derived organoids model treatment response of metastatic gastrointestinal cancers. Science, 359(6378), 920-926.
Walsh, A. J., Castellanos, J. A., Nagathihalli, N. S., Merchant, N. B., & Skala, M. C. (2016). Optical imaging of drug-induced metabolism changes in murine and human pancreatic cancer organoids reveals heterogeneous drug response. Pancreas, 45(6), 863-869.
Watanabe, H., Ma, Q., Peng, S., Adelmant, G., Swain, D., Song, W., . . . Bass, A. J. (2014). SOX2 and p63 colocalize at genetic loci in squamous cell carcinomas. Journal of Clinical Investigation, 124(4), 1636-1645.
Wels J, Kaplan R N, Rafii S, Lyden D. Migratory neighbors and distant invaders: tumor-associated niche cells. Genes Dev. 2008; 22(5):559-74. Epub 2008/03/05. doi: 22/5/559 [pii] 10.1101/gad.1636908. PubMed PMID: 18316475; PMCID: 2731657.
Whelan, K. A., Chandramouleeswaran, P. M., Tanaka, K., Natsuizaka, M., Guha, M., Srinivasan, S., . . . Nakagawa, H. (2017). Autophagy supports generation of cells with high CD44 expression via modulation of oxidative stress and Parkin-mediated mitochondrial clearance. Oncogene, 36(34), 4843-4858.
Whelan, K. A., Merves, J. F., Giroux, V., Tanaka, K., Guo, A., Chandramouleeswaran, P. M., . . . Nakagawa, H. (2017). Autophagy mediates epithelial cytoprotection in eosinophilic oesophagitis. Gut, 66(7), 1197-1207.
Whelan, K. A., Muir, A. B., & Nakagawa, H. (2018). Esophageal 3D culture systems as modeling tools in esophageal epithelial pathobiology and personalized medicine. Cellular and Molecular Gastroenterology and Hepatology, 5(4), 461-478.
Zambelli V O, Gross E R, Chen C H, Gutierrez V P, Cury Y, Mochly-Rosen D. Aldehyde dehydrogenase-2 regulates nociception in rodent models of acute inflammatory pain. Sci Transl Med. 2014; 6(251):251ra118. Epub 2014/08/29. doi: 10.1126/scitranslmed.3009539. PubMed PMID: 25163478; PMCID: PMC4234033.
Zhao J S, Li W J, Ge D, Zhang P J, Li J J, Lu C L, Ji X D, Guan D X, Gao H, Xu L Y, Li E M, Soukiasian H, Koeffler H P, Wang X F, Xie D. Tumor initiating cells in esophageal squamous cell carcinomas express high levels of CD44. PLoS One. 2011; 6(6):e21419. doi: 10.1371/journal.pone.0021419. PubMed PMID: 21731740; PMCID: PMC3123317.
Zoller M. CD44: can a cancer-initiating cell profit from an abundantly expressed molecule? Nat Rev Cancer. 2011; 11(4):254-67. Epub 2011/03/11. doi: nrc3023 [pii] 10.1038/nrc3023. PubMed PMID: 21390059.

All documents cited in this application are hereby incorporated by reference as if recited in full herein.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

What is claimed is:

1. A method for stratifying the risk of developing a tumor in a subject, comprising:
a) obtaining a biological sample from the subject;
b) generating a three-dimensional (3D) organoid system from the biological sample; and
c) detecting one or more dysplastic 3D structures.

2. The method of claim 1, wherein the tumor is an oral squamous cell carcinoma (OSCC).

3. The method of claim 1, further comprising the steps of:
a) determining the aldehyde dehydrogenase (Aldh)-2 genotype of the subject using the 3D organoid system;
b) identifying the subject as having high risk of developing the cancer, if the Aldh2 genotype is Aldh2^{E487K}; and
c) initiating a therapeutic protocol that prevents the progression of the tumor.

4. The method of claim 1, wherein the subject is human.

5. The method of claim 1, wherein the subject has oral preneoplasia.

6. The method of claim 1, wherein the biological sample is originating in oral, pharyngeal or esophageal mucosa.

7. A method for treating or ameliorating the effects of a tumor in a subject, comprising:
a) obtaining a biological sample from the subject
b) generating a three-dimensional (3D) organoid system from the biological sample;
c) determining the aldehyde dehydrogenase (Aldh)-2 genotype of the subject using the 3D organoid system; and
d) administering to the subject with an effective amount of a chemotherapy agent, if the Aldh2 genotype is Aldh2^{E487K}.

8. The method of claim 7, wherein the tumor is an oral squamous cell carcinoma (OSCC).

9. The method of claim 7, wherein the biological sample is an originating in oral, pharyngeal or esophageal mucosa.

10. The method of claim 7, wherein the chemotherapy agent is selected from the group consisting of actinomycin all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil (5FU), gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and combinations thereof.

11. The method of claim 7, wherein the chemotherapy agent is selected from cisplatin, fluorouracil (5FU), and combinations thereof.

12. A method for improving the efficacy of chemotherapy in a subject with a tumor, comprising:
a) obtaining a biological sample from the subject;
b) generating a three-dimensional (3D) organoid system from the biological sample;
c) determining the aldehyde dehydrogenase (Aldh)-2 genotype of the subject using the 3D organoid system; and
d) co-administering to the subject with an effective amount of a chemotherapy agent and an effective amount of an agent that inhibits Aldh2, if the Aldh2 genotype is Aldh2^{E487K}.

13. The method of claim 12, wherein the tumor is the cancer oral squamous cell carcinoma (OSCC).

14. The method of claim 12, wherein the biological sample is an originating oral, pharyngeal or esophageal mucosa.

15. The method of claim 12, wherein the chemotherapy agent is selected from the group consisting of actinomycin all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil (5FU), gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and combinations thereof.

16. The method of claim 12, wherein the chemotherapy agent is selected from cisplatin, fluorouracil (5FU), and combinations thereof.

17. The method of claim 12, wherein the agent that inhibits Aldh2 is selected from the group consisting of ampal, benomyl, citral, chloral hydrate, chlorpropamide, coprine, cyanamide, daidzin, CVT-10216, DEAB, DPAB, disulfiram, gossypol, kynurenine tryptophan metabolites, molinate, nitroglycerin, pargyline, and combinations thereof.

18. The method of claim 12, wherein the agent that inhibits Aldh2 is disulfiram.

19. The method of claim 12, wherein the agent that inhibits Aldh2 is administered to the subject before, concurrent with or after the administration of the chemotherapy agent.

20. A method of treating or ameliorating the effects of an oral tumor in a subject comprising the steps of:
a) detecting the presence of the aldehyde dehydrogenase 2 (Aldh2) single nucleotide polymorphism (SNP) Aldh2^{E487K} in the subject; and
b) administering a chemotherapy agent if Aldh2^{E487K} is detected.

21. The method of claim 20, wherein the chemotherapy agent is selected from the group consisting of actinomycin all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil (5FU), gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and combinations thereof.

22. The method of claim 20, wherein the chemotherapy agent is selected from cisplatin, 5FU, and combinations thereof.

23. A method of treating or ameliorating the effects of an oral tumor comprising the steps of:
a) detecting the presence of Aldh2^{E487K}; and
b) administering an Aldh2 inhibitor and a chemotherapy agent.

24. The method of claim 23, wherein the agent that inhibits Aldh2 is selected from the group consisting of ampal, benomyl, citral, chloral hydrate, chlorpropamide, coprine, cyanamide, daidzin, CVT-10216, DEAB, DPAB, disulfiram, gossypol, kynurenine tryptophan metabolites, molinate, nitroglycerin, pargyline, and combinations thereof.

25. The method of claim 23, wherein the chemotherapy agent is selected from the group consisting of actinomycin all-trans retinoic acid, azacitidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil (5FU), gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, vindesine, and combinations thereof.

26. A method of screening for efficacy of a chemotherapy agent against a tumor comprising the steps of:
a) obtaining a biological sample from a subject;
b) generating a three-dimensional (3D) organoid system from the biological sample; and
c) contacting one or more cells of the 3D organoid system with one or more chemotherapy agents to detect efficacy against the tumor.

27. The method of claim 26, further comprising the step of detecting the presence of the aldehyde dehydrogenase 2 (Aldh2) single nucleotide polymorphism (SNP) Aldh2^{E487K}.
